(19) United States
(12) Patent Application Publication
(10) Pub. No.: US 2023/0090771 A1
(43) Pub. Date: Mar. 23, 2023

(54) NOVEL METHODS FOR CREATING ALPHA-N-METHYLATED POLYPEPTIDES

(71) Applicant: REGENTS OF THE UNIVERSITY OF MINNESOTA, MINNEAPOLIS, MN (US)

(72) Inventor: Michael F. FREEMAN, Minneapolis, MN (US)

(73) Assignee: REGENTS OF THE UNIVERSITY OF MINNESOTA, MINNEAPOLIS, MN (US)

(21) Appl. No.: 17/801,458

(22) PCT Filed: Feb. 22, 2021
(86) PCT No.: PCT/US2021/019009

§ 371 Date: Aug. 22, 2022

Related U.S. Application Data
(60) Provisional application No. 62/979,947, filed on Feb. 21, 2020.

(51) Int. Cl. C07K 14/375; C12N 9/10; C12P 21/02; C12N 15/70

(52) U.S. Cl. CPC C07K 14/375; C12N 9/1007; C12P 21/02; C12N 15/70; C07K 2319/21; C07K 2319/23; C07K 2319/43

(57) ABSTRACT

Provided herein are methods and compositions for producing alpha-N-methylated peptides in vitro and in vivo. This disclosure also provides in vivo and in vitro methods for producing highly diverse alpha-N-methylated peptide libraries by methylating natural or non-natural alpha-N-methyltransferase target peptides.

### CROSS REFERENCE TO RELATED APPLICATIONS

This application claims priority to U.S. Provisional Application No. 62/979,947 filed on Feb. 21, 2020, the contents of which are incorporated by reference in their entireties.

### STATEMENT REGARDING FEDERALLY SPONSORED RESEARCH

This invention was made with government support under GM133475 awarded by the National Institutes of Health. The government has certain rights in the invention.

### SEQUENCE LISTING

A Sequence Listing accompanies this application and is submitted as an ASCII text file of the sequence listing named “920171_00406_ST25.txt” which is 319 KB in size and was created on Feb. 18, 2021. The sequence listing is electronically submitted via EFS-Web with the application and is incorporated herein by reference in its entirety.

### BACKGROUND

Alpha-N-methylations on peptide and protein amide backbones engender unique physiochemical properties to peptides and polypeptides that include increased proteolytic stability, cell membrane permeability, and restricted structural flexibility in comparison to the corresponding non-methylated peptides and polypeptides. Chemical synthesis of alpha-N-methylated polypeptides is expensive as the methods are limited by lower amino acid coupling yields and limits to scale of production. In biological systems, alpha-N-methylated peptides can be made by large nonribosomal peptide synthetase enzyme-encoding pathways, such as for cyclosporine, or through ribosomally encoded and post-translationally modified peptide (RiPP) pathways, such as the omphalotins and the gymnopeptides. Nonribosomal peptide synthetases (NRPSs) are large multimodular and multidomain-containing enzymes. NRPSs require a dedicated minimal enzyme domain complex of ˜250 kDa for each individual amino acid into a polypeptide, thus making these enzymes very difficult to engineer to produce different alpha-N-methylated polypeptides. RiPP pathways have several advantages for the production of alpha-N-methylated peptides, since the polypeptides are genetically encoded and first transcribed and translated into polypeptide precursors. The polypeptide precursors are typically composed of a short N-terminal leader sequence and a C-terminal core peptide; the core peptide is destined to become the modified polypeptide metabolite. The core peptide sequence is post-translationally modified, such as with alpha-N-methylations, on the peptide/polypeptide backbone. Since the polypeptide precursors are genetically encoded, the sequences can be easily engineered to create different alpha-N-methylated polypeptides.

Currently, the only known family of RiPPs to produce alpha-N-methylated polypeptides are called the borosins; examples of which include the omphalotins and gymnopeptides. The polypeptide precursor encoding the omphalotins and gymnopeptides are translated as ˜400 amino acid polypeptides that encode the alpha-N-methyltransferase within the same amino acid sequence as the core peptide. The borosin core peptide is post-translationally modified with alpha-N-methylations and in some cases other modifications. Ultimately, the C-terminal post-translationally modified sequence of the polypeptide encoding the alpha-N-methyltransferase is cleaved off to yield the mature alpha-N-methylated natural product. The borosin alpha-N-methyltransferases work in trans, as the enzyme is a homodimer, with the subunit A methylating the C-terminus of subunit B, and vice versa. However, the current borosin RiPP systems are plagued by slow reaction times (kcatApp of ˜0.32 methylations per hour) and single-substrate turnover due to the fused core sequence to the alpha-N-methyltransferases. Accordingly, there remains a need in the field for improved borosin alpha-N-methyltransferase systems and improved methods for producing alpha-N-methylated peptides, preferably methods having faster reaction times and capable of multiple substrate turnover.

### SUMMARY OF THE INVENTION

Provided herein are improved methods for producing alpha-N-methylated peptides, where the methods are faster and, unlike conventional methods, are capable of multiple substrate turnover. The methods, compositions, and systems of this disclosure find utility in engineering selectively alpha-N-methylated products for academic and commercial applications.

In a first aspect, provided herein is a method for producing an alpha-N-methylated peptide. The method can comprise or consist essentially of contacting a split borosin alpha-N-methyltransferase protein to a target peptide, and incubating the split borosin alpha-N-methyltransferase protein and the target peptide in the presence of a methyl donor to produce an alpha-N-methylated target peptide. The split borosin alpha-N-methyltransferase can comprise an amino acid sequence having at least 70% sequence similarity to an amino acid sequence selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15. The split borosin alpha-N-methyltransferase can comprise an amino acid sequence having at least 90% sequence similarity to an amino acid sequence selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15. The split borosin alpha-N-methyltransferase can comprise an amino acid sequence selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15. The target peptide can be a split borosin precursor comprising an amino acid sequence having at least 70% sequence similarity to an amino acid sequence selected from SEQ ID NO:2, SEQ ID NO:4, SEQ ID NO:6, SEQ ID NO:8, SEQ ID NO:10, SEQ ID NO:12, SEQ ID NO:14, and SEQ ID NO:16. The methyl donor can be S-Adenosylmethionine (SAM) or an analog thereof. In some embodiments, the method is in vitro and the split borosin alpha-N-methyltransferase and target peptide are isolated proteins. One or more of the split borosin alpha-N-methyltransferase and target peptide can be a recombinant protein or synthetic protein. In some embodiments, the isolated split borosin methyltransferase protein is obtained by introducing into a cell an exogenous expression vector comprising a nucleotide sequence encoding a split borosin methyltransferase protein, expressing the split borosin methyltransferase protein in the cell, and purifying the expressed split borosin methyltransferase protein. In other embodiments, the method is in vivo and contacting of the split borosin alpha-N-methyltransferase to the target peptide can occur in a host cell. The method can further comprise introducing into a cell one or more expression vectors encoding the split borosin methyltransferase protein and the target peptide.

In another aspect, provided herein is an in vivo method of producing a peptide library comprising random alpha-N-methylated peptides, the method comprises introducing into a cell one or more expression vectors comprising a nucleotide sequence encoding a split borosin alpha-N-methyltransferase and one or more nucleotide sequences encoding one or more split borosin precursors, wherein the one or more nucleotide sequences encoding the one or more split borosin precursors comprise one or more genetic variation relative to a nucleotide sequence encoding a wild-type split borosin precursor; optionally detecting production of alpha-N-methylated peptides; and isolating the alpha-N-methylated peptides to produce the peptide library. The one or more genetic variations can be introduced by random mutagenesis. The one or more genetic variations can be introduced by site-directed mutagenesis. The nucleotide sequence encoding the split borosin alpha-N-methyltransferase can be in cis to the nucleotide sequence(s) encoding the split borosin precursor(s). The nucleotide sequence encoding the split borosin alpha-N-methyltransferase can be in trans to the nucleotide sequence(s) encoding the split borosin precursor(s). The split borosin alpha-N-methyltransferase can comprise an amino acid sequence having at least 70% sequence similarity to an amino acid sequence selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15. The split borosin alpha-N-methyltransferase can comprise an amino acid sequence selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15. The wild-type split borosin precursor can comprise an amino acid sequence having at least 70% sequence similarity to an amino acid sequence selected from SEQ ID NO:2, SEQ ID NO:4, SEQ ID NO:6, SEQ ID NO:8, SEQ ID NO:10, SEQ ID NO:12, SEQ ID NO:14, and SEQ ID NO:16. The wild-type split borosin precursor can comprise an amino acid sequence having at least 70% sequence similarity to an amino acid sequence selected from SEQ ID NO:2, SEQ ID NO:4, SEQ ID NO:6, SEQ ID NO:8, SEQ ID NO:10, SEQ ID NO:12, SEQ ID NO:14, and SEQ ID NO:16. One or more of the split borosin alpha-N-methyltransferase and the one or more split borosin precursors can comprise an affinity tag and/or a solubility tag.

In another aspect, provided herein is an in vitro method of producing a peptide library comprising random alpha-N-methylated peptides. The method can comprise or consist essentially of (a) contacting an isolated split borosin alpha-N-methyltransferase to one or more split borosin precursors comprising one or more genetic variations relative to a wild-type split borosin precursor in the presence of a methyl donor to produce one or more alpha-N-methylated split borosin precursors, and (b) isolating the alpha-N-methylated split borosin precursor peptides to produce the peptide library. In some embodiments, the method further comprises optionally detecting production of alpha-N-methylated peptides. In some embodiments, the one or more split borosin precursors are obtained by: (i) introducing into a cell an expression vector comprising one or more nucleotide sequences encoding the one or more split borosin precursors comprising one or more genetic variations relative to a nucleotide sequence encoding a wild-type split borosin precursor, (ii) expressing the one or more split borosin precursors in the cell, and (iii) purifying the expressed one or more split borosin precursors. The one or more genetic variations can be introduced by random mutagenesis. The one or more genetic variations can be introduced by site-directed mutagenesis.

In another aspect, provided herein is a vector comprising a nucleotide sequence encoding a split borosin alpha-N-methyltransferase domain and a heterologous promoter. The nucleotide sequence can encode a split borosin alpha-N-methyltransferase domain comprising an amino acid sequence having at least 70% sequence similarity to an amino acid sequence selected from SEQ ID NO:1, SEQ ID NO:3, SEQ ID NO:5, SEQ ID NO:7, SEQ ID NO:9, and SEQ ID NO:11, SEQ ID NO:13, and SEQ ID NO:15. The nucleotide sequence can encode a split borosin alpha-N-methyltransferase domain comprising an amino acid selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15.

In another aspect, provided herein is a host cell comprising a vector of this disclosure. The cell can be a prokaryotic cell.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic illustration of overall protein architecture of members of the borosin family. The three canonical borosin protein architectures are single proteins comprising an alpha-N-methyltransferase domain (orange box), the core peptide region(s) that are alpha-N-methylated and destined to be the polypeptide products (yellow boxes), and a linker region of varying sequence length and composition (grey box). The color peach represents the N-methyltransferase domains (NMT) that are encoded in the bacterial split borosin systems. Light blue boxes (TPR) denotes a tetratricopeptide repeat domain of uncharacterized function, which may possibly be involved in small molecule or protein-protein interactions, and green boxes (GGDEF) denote GGDEF domains of uncharacterized function, which may possibly harbordiguanylate cyclase activity.

FIG. 2 presents phylogenetic analysis of borosin alpha-N-methyltransferase domains as compared to split borosin alpha-N-methyltransferase domains. A: A MAFFT protein sequence alignment for a selection of sequence-trimmed borosin and split borosin alpha-N-methyltransferase domains. Ordered from top to bottom, the sequences shown correspond to SEQ ID NO:125-SEQ ID NO:229. Fungal borosins are marked on the left with a blue box, archaeal sequences are marked with a green box, and bacterial sequences are marked with a red box. The respective percent sequence identity/similarity of OphMA to the split borosin alpha-N-methyltransferase domains that are described in this patent are: SonM 38.8%/57.6%; StrM 37.9%/52.7%; RceM 48.6%/57.6%; 50.4%/69.4%; AinM 47.1%/66.1%; PmoM 48.3%/69.4%. B. Baysian phylogenetic analysis (MRBAYES; 1,000,000 replicates; posterior probabilities listed) of the borosins and split borosins aligned in panel A, with the yellow-type-labelled outgroup CobA. Fungi-derived borosins are outlined in blue, archaeal sequences are outlined in green, and the bacterial borosins are outlined in red. The split borosins that are examples in this patent are boxed in yellow. Fungal sequences clade separately from the six split borosin types in bacteria and archaea. For both panels: the names of the proteins are colored coded based on their family and/or structural type. Grey text refer to fungal borosins as well as borosins not yet categorized into a structural type, type I split borosins are in black text, type II split borosins in blue, type III are in red, type VI in orange, type V in purple, and type VI in green.

FIG. 3 illustrates a genetic locus and methylation pattern for the Type I split borosin SonM and SonA in Shewanella oneidensis. A. Block arrows represent genes within and/or surrounding the putative split borosin gene cluster. Protein IDs as well as the proposed functions of all genes are listed. In addition, a summary of the alpha-N-methylations observed by tandem liquid chromatographic and mass spectrometric analysis (LC-MS/MS) of the core region on the split borosin precursor SonA. B. The protein sequences of SonM and SonA; these do not include supplementary affinity and solubility tags used for polypeptide purification purposes.

FIG. 4 demonstrates purification of SonM-SonA complex from over-expression in Escherichia coli. A. SDS-PAGE analysis of fractions collected during expression and nickel affinity purification of SonM and SonA in E. coli. B. Profile of the purified protein from elutions in panel A on size exclusion chromatography. C. SDS-PAGE analysis of peaks shown in panel B. D. MS1 alpha-N-methylation profiles for SonA from panel C. This reveals the vast majority of SonA is purified as the 2-alpha-N-methylated state.

FIG. 5 demonstrates LC-MS/MS results of in vivo co-expression of SonM and SonA. A-C. Alpha-N-methylations are observed as mass shifts in multiple fragments of the SonA core. Analysis of the mass spectra, shown as cartoon boxes overlaying the observed peptide fragment, are shown above the LC-MS/MS data.

FIG. 6 presents crystallographic comparison of the dimer of hetero-dimers formed by the SonM-SonA complex, in comparison to the canonical borosin homodimer, OphMA. A. Structural overview in cartoon overlaid with semi-transparent surface representation of the SonM-SonA complex (left) versus the OphMA structure (right). Each color represents a different subunit (i.e., different protein monomer) in the complexes (SonM proteins are in grey and tan, SonA proteins are in turquoise and purple; OphMA proteins are in yellow and brown). Above each structure is a two-dimensional cartoon describing the structures. B. Zoom-in of two different orientations (45 degree rotation) for the active site of one set of SonA-SonM heterodimers, revealing alpha-N-methylations on L63 and 165 on the SonA core peptide (colored orange). S-Adenosylmethionine (SAM) is the methyl donor for the reaction.

FIG. 7 presents a summary of kinetic data for in vitro reactions of SonM and SonA with SAM. A. SonM wild type (WT) and several active-site mutants reveal multiple SonA substrate turnover. Michaelis-Menton kinetics data where SAM was kept in excess while the concentration of SonA was varied. B. Michaelis-Menton kinetics data where SonA was kept in excess while the concentration of SAM was varied. C. Table showing alpha-N-methylation activity of SonM on a variety of SonA mutants. Substrate turnover numbers (kcat) are listed as well as the methylation pattern and overall relative methylation for an extended (16 hour) incubation with SonM. D-BB. LC-MS/MS results of in vivo co-expression of the SonM and SonA variants listed in B. Alpha-N-methylations are observed as mass shifts in multiple fragments of the SonA core. Analysis of the mass spectra, shown as cartoon boxes overlaying the observed peptide fragment, are shown above the LC-MS/MS data.

FIG. 8 presents rate vs. substrate concentration graphs for kinetics data tabulated in FIG. 7. A. SonM WT curves when varying the concentrations of SonA (left) and SAM (right). B. SonM mutant Y93F curves when varying the concentrations of SonA (left) and SAM (right). C. SonM mutant Y58F curves when varying the concentrations of SonA (left) and SAM (right). D. SonM mutant R67K curves when varying the concentrations of SonA (left) and SAM (right). E. SonM mutant Y71F curves when varying the concentrations of SonA (left) and SAM (right).

FIG. 9 illustrates a genetic locus and methylation pattern for the Type I split borosin StrM and StrA in Streptomyces sp. NRRL S-118. A. Block arrows represent genes within and/or surrounding the putative split borosin gene cluster. Protein IDs as well as the proposed functions of all genes are listed. In addition, a summary of the alpha-N-methylations observed by LC-MS/MS of the core region on the split borosin precursor StrA. B. The protein sequences of StrM and StrA; these do not include supplementary affinity and solubility tags used for polypeptide purification purposes.

FIG. 10 presents LC-MS/MS results of in vitro reactions of E. coli-overexpressed and purified StrM and StrA. A-E. Alpha-N-methylations are observed as mass shifts in multiple fragments of the StrA core. Analysis of the mass spectra, shown as cartoon boxes overlaying the observed peptide fragment, are shown above the LC-MS/MS data.

FIG. 11 presents a genetic locus and methylation pattern for the Type II split borosin RceM and RceA in Rhodospirillum centenum SW. A. Block arrows represent genes within and/or surrounding the putative split borosin gene cluster. Protein IDs as well as the proposed functions of all genes are listed. In addition, a summary of the alpha-N-methylations observed by LC-MS/MS of the core region on the split borosin precursor RceA. B. The protein sequences of RceM and RceA; these do not include supplementary affinity and solubility tags used for polypeptide purification purposes.

FIG. 12 presents LC-MS/MS results of in vivo co-expression of RceM and RceA. A-I. Alpha-N-methylations are observed as mass shifts in multiple fragments of the RceA core. Analysis of the mass spectra, shown as cartoon boxes overlaying the observed peptide fragment, are shown above the LC-MS/MS data. Note that several replicates of putative RceA cores are present in the sequence and thus it cannot be resolved which sequence replicate is represented by these data.

FIG. 13 presents a genetic locus and methylation pattern for the Type III split borosin BstM and BstA in Burkholderia stabilis. A. Block arrows represent genes within and/or surrounding the putative split borosin gene cluster. Protein IDs as well as the proposed functions of all genes are listed. In addition, a summary of the alpha-N-methylations observed by LC-MS/MS of the core region on the split borosin precursor BstA. The depicted pattern is a compilation of peptide fragments showing methylation at those positions. B. The protein sequences of BstM and BstA; these do not include supplementary affinity and solubility tags used for polypeptide purification purposes.

FIG. 14 presents LC-MS/MS results of in vivo co-expression of BstM and BstA. A-D. Alpha-N-methylations are observed as mass shifts in multiple fragments of the BstA core. Analysis of the mass spectra, shown as cartoon boxes overlaying the observed peptide fragment, are shown above the LC-MS/MS data.

FIG. 15 presents genetic locus and methylation pattern for the Type IV split borosin AinM and AinA in Achromobacter insuavis. A. Block arrows represent genes within and/or surrounding the putative split borosin gene cluster. Protein IDs as well as the proposed functions of all genes are listed. In addition, a summary of the alpha-N-methylations observed by LC-MS/MS of the core region on the split borosin precursor AinA. The depicted pattern is a compilation of peptide fragments showing methylation at those positions. B. The protein sequences of AinM and AinA; these do not include supplementary affinity and solubility tags used for polypeptide purification purposes.

FIG. 16 presents LC-MS/MS results of in vivo co-expression of AinM and AinA. A-D. Alpha-N-methylations are observed as mass shifts in multiple fragments of the AinA core. Analysis of the mass spectra, shown as cartoon boxes overlaying the observed peptide fragment, are shown above the LC-MS/MS data.

FIG. 17 presents genetic locus and methylation pattern for the Type V split borosin PmoM and PmoA in Pseudomonas mosselii. A. Block arrows represent genes within and/or surrounding the putative split borosin gene cluster. Protein IDs as well as the proposed functions of all genes are listed. In addition, a summary of the alpha-N-methylations observed by LC-MS/MS of the core region on the split borosin precursor PmoA. The depicted pattern is a compilation of peptide fragments showing methylation at those positions. B. The protein sequences of PmoM and PmoA; these do not include supplementary affinity and solubility tags used for polypeptide purification purposes.

FIG. 18 presents LC-MS/MS results of in vivo co-expression of PmoM and PmoA. A-C. Alpha-N-methylations are observed as mass shifts in multiple fragments of the PmoA core. Analysis of the mass spectra, shown as cartoon boxes overlaying the observed peptide fragment, are shown above the LC-MS/MS data.

FIG. 19 presents a genetic locus and methylation pattern for the Type III split borosin BlaM and BlaA in Brevibacillus laterosporus PE36. A. Block arrows represent genes within and/or surrounding the putative split borosin gene cluster. Protein IDs as well as the proposed functions of all genes are listed. In addition, a summary of the alpha-N-methylations observed by LC-MS/MS of the core region on the split borosin precursor BlaA. The depicted pattern is a compilation of peptide fragments showing methylation at those positions. B. The protein sequences of BlaM and BlaA; these do not include supplementary affinity and solubility tags used for polypeptide purification purposes.

FIG. 20 presents LC-MS/MS results of in vivo co-expression of BlaM and BlaA. A-C. Alpha-N-methylations are observed as mass shifts in multiple fragments of the BlaA core. Analysis of the mass spectra, shown as cartoon boxes overlaying the observed peptide fragment, are shown above the LC-MS/MS data.

FIG. 21 presents a genetic locus and methylation pattern for the Type III split borosin BlaM and BlaA in Brevibacillus laterosporus PE36. A. Block arrows represent genes within and/or surrounding the putative split borosin gene cluster. Protein IDs as well as the proposed functions of all genes are listed. In addition, a summary of the alpha-N-methylations observed by LC-MS/MS of the core region on the split borosin precursor BlaA. The depicted pattern is a compilation of peptide fragments showing methylation at those positions. B. The protein sequences of BlaM and BlaA; these do not include supplementary affinity and solubility tags used for polypeptide purification purposes.

FIG. 22 presents LC-MS/MS results of in vivo co-expression of BlaM and BlaA. A-C. Alpha-N-methylations are observed as mass shifts in multiple fragments of the BlaA core. Analysis of the mass spectra, shown as cartoon boxes overlaying the observed peptide fragment, are shown above the LC-MS/MS data.

### DETAILED DESCRIPTION

The methods and compositions of this disclosure are based at least in part on the inventors' identification and characterization of a distantly related subfamily of borosin peptides. The term “borosin” is used to describe a family of ribosomally synthesized and posttranslationally modified peptides (RiPPs) that are alpha-N-methylated in the amide backbone of pepitdes. Canonical borosins are expressed as precursor polypeptides that comprise both the “core peptide” (i.e., the peptide product to be alpha-N-methylated) and the alpha-N-methyltransferase (i.e., the enzyme responsible for the alpha-N-methylation). Ultimately, the C-terminal, post-translationally modified sequence of the polypeptide is cleaved off to yield a mature alpha-N-methylated peptide product. The present inventors discovered a novel class of borosins that are referred to herein as “split borosins,” which are predominantly found in bacteria and archaea. In general, the alpha-N-methyltransferase domains of the split borosins have less than 70% amino acid identity to OphMA (also referred to as OphA), which is the canonical, first characterized borosin alpha-N-methyltransferase. Importantly, these methyltransferases install alpha-N-methylations on precursor peptides that are encoded on a separate gene (i.e., a gene that does not also encode an alpha-N-methyltransferase). Since the split borosin precursor genes of this disclosure are not fused to the N-methyltransferase, the precursor peptide can be modified in trans. As a result, the split borosin precursor peptides can be expressed and purified prior to their modification. Thus, the methods and systems of this disclosure provide a more genetically tractable method of engineering selectively alpha-N-methylated products for academic and commercial applications. Advantageously, the split borosins of this disclosure are capable of multiple substrate turnover, making the methods and compositions of this disclosure markedly different than those utilizing canonical borosin pathways. As described in this disclosure, these advantageous enzymatic attributes are supported by evidence including phylogenetic analyses, enzyme-precursor complex crystallographic data, enzyme kinetics data, and mass-spectrometric verification of alpha-N-methylated polypeptides

In a first aspect, provided herein are methods for producing alpha-N-methylated peptides. The term “alpha-N-methylated peptides” refers to peptides that have been methylated on α-amino group(s) in amides of the peptide backbone. In some cases, the method comprises contacting a split borosin alpha-N-methyltransferase to a target peptide to be alpha-N-methylated in the presence of a methyl donor to produce an alpha-N-methylated target peptide.

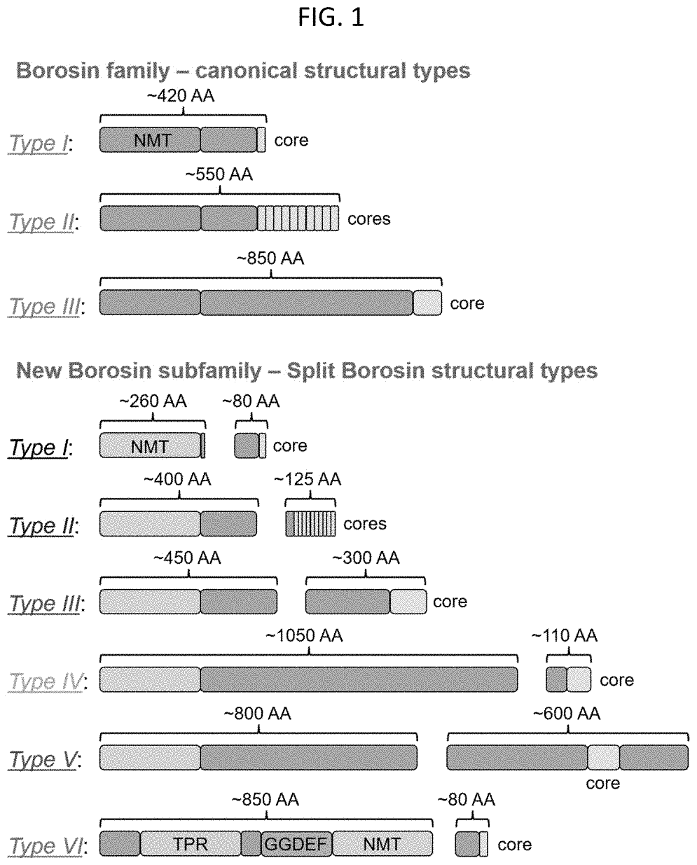

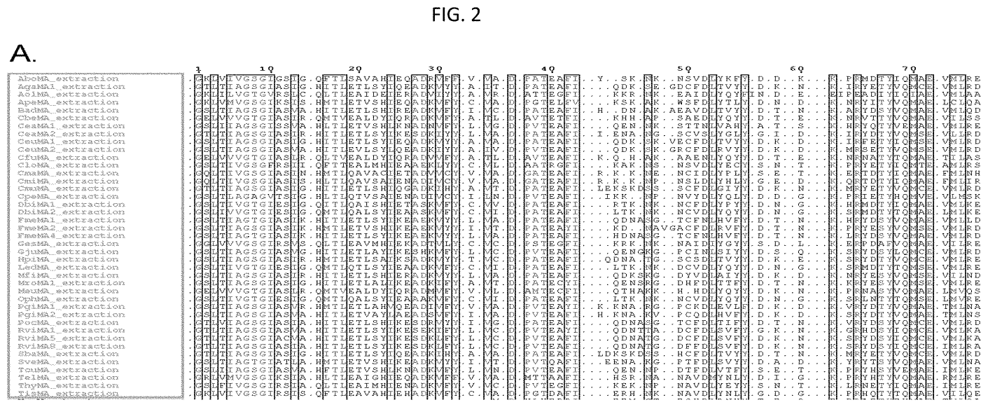

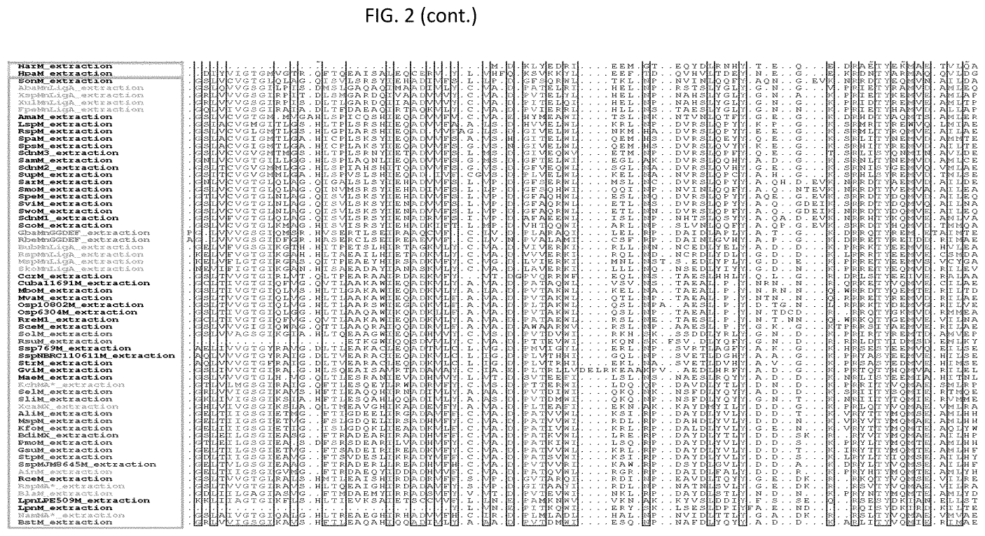

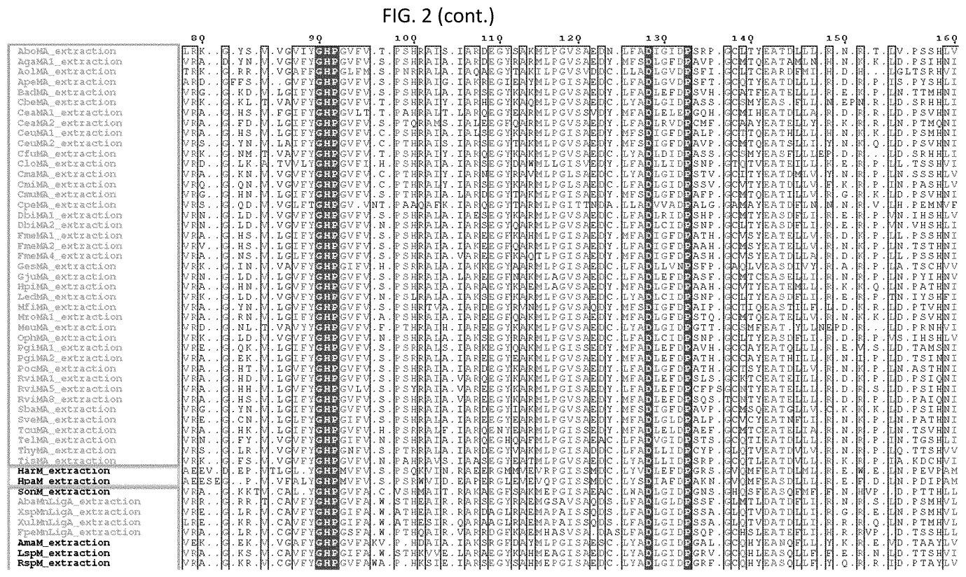

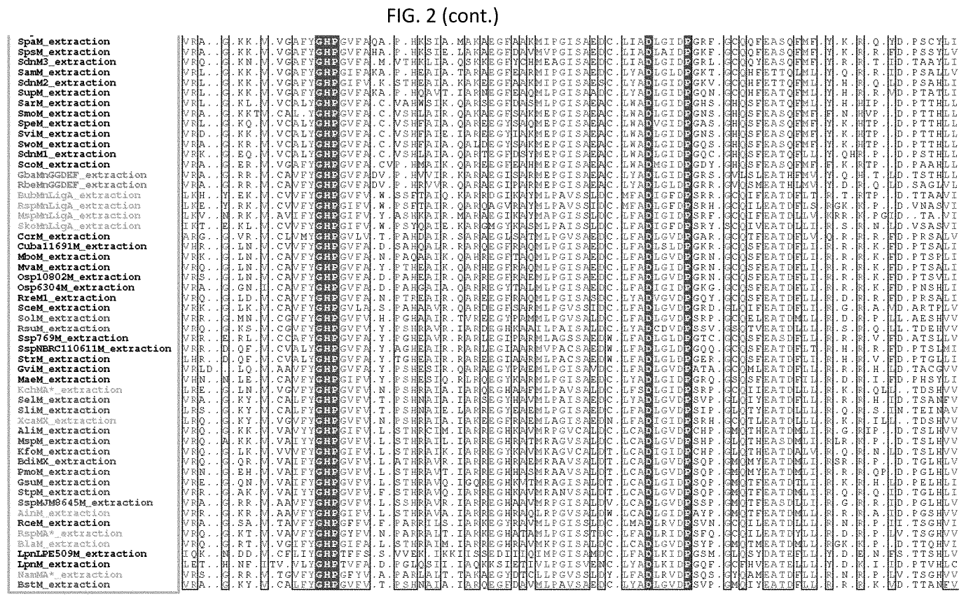

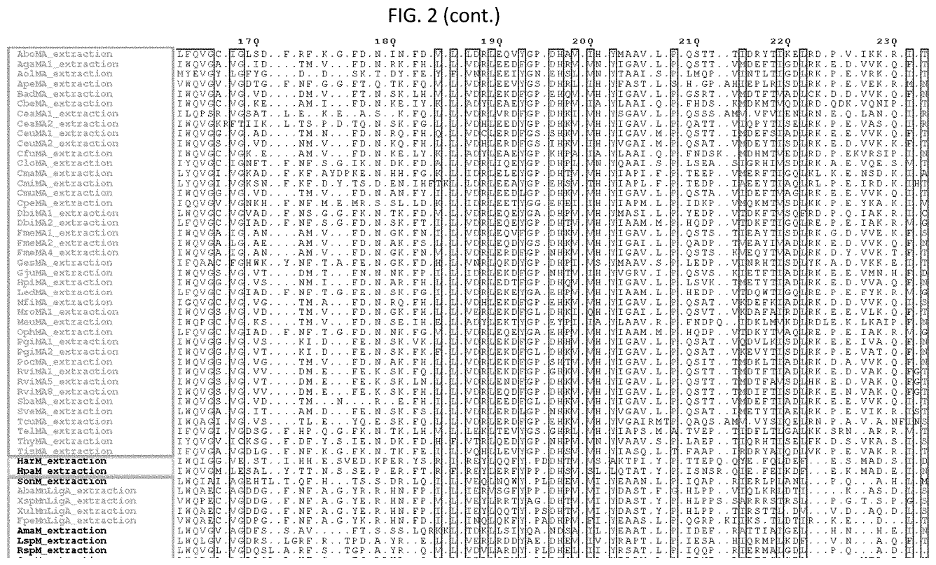

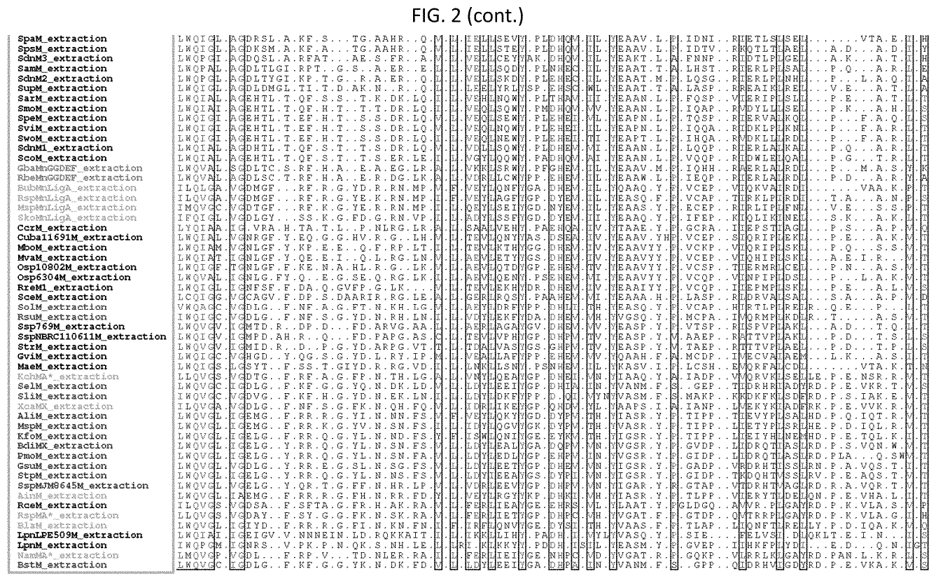

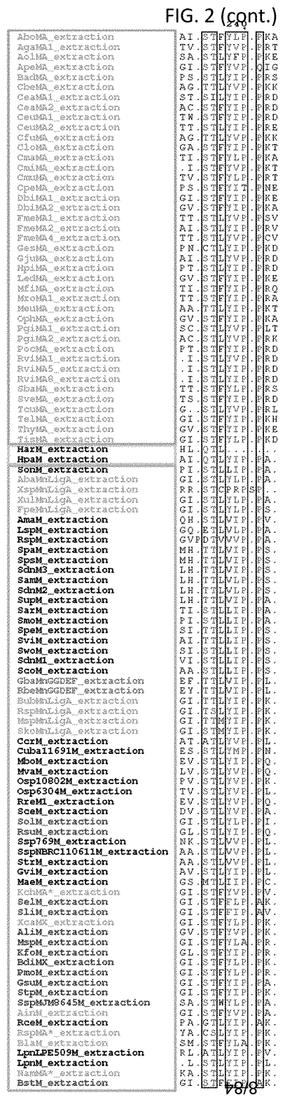

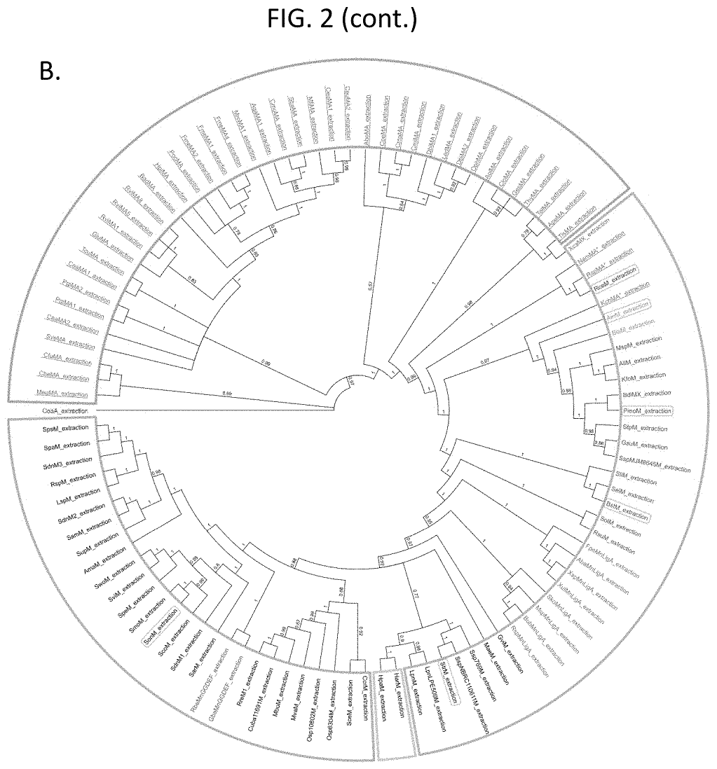

The alpha-N-methyltransferases described herein are enzymes that methylate target peptides containing a particular target motif. A “split borosin alpha-N-methyltransferase” is an alpha-N-methyltransferase that is expressed as part of a split borosin pathway, whereas a “split borosin precursor” is a target peptide that is expressed as part of a split borosin pathway. The minimal target motif of these enzymes is any amide nitrogen in a peptide backbone. As illustrated in FIG. 1, the architectures of the split borosin proteins are distinct from canonical borosins in two main aspects: (1) the alpha-N-methyltransferase domains are less than 70% identical to the canonical borosin OphMA domain, and (2) the precursor peptide (which contains one or more core peptide, shown in yellow) and the alpha-N-methyltransferase are expressed as separate polypeptides. Thus, the precursor peptide and the alpha-N-methyltransferase may be encoded either by a single nucleotide sequence (i.e., a polycistronic sequence) or by multiple, separate nucleotide sequences.

Six distinct structural types of split borosins (Types I-VI) have been identified based on the overall protein architectures of the alpha-N-methyltransferase domain-containing protein and the precursor proteins. Exemplary Type I-V split borosins are presented in Table 1 as alpha-N-methyltransferase polypeptide and precursor polypeptide pairs.

TABLE 1
Amino Acid Sequences of Representative Split Borosins

| Split | Name and Organism | |
|---|---|---|
| Borosin | with protein accession | |
| Type | ID | Amino Acid Sequence |
| Type 1 | split borosin alpha-N- | MGSLVCVGTGLQLAGQISVLSRSYIEHADIVFSLLP |
| | methyltransferase | DGFSQRWLTKLNPNVINLQQFYAQNGEVKNRRDT |
| | WP_011071665 | YEQMVNAILDAVRAGKKTVCALYGHPGVFACVSH |
| | SonM (Shewanella | MAITRAKAEGFSAKMEPGISAEACLWADLGIDPGN |
| | oneidensis) | SGHQSFEASQFMFFNHVPDPTTHLLLWQIAIAGEHT |
| | | LTQFHTSSDRLQILVEQLNQWYPLDHEVVIYEAAN |
| | | LPIQAPRIERLPLANLPQAHLMPISTLLIPPAKKL |
| | | EYNYAILAKLGIGPEDLG (SEQ ID NO: 1) |
| Type 1 | split borosin precursor | MSGLSDFFTQLGQDAQLMEDYKQNPEAVMRAHGL |
| | WP_011071666 | TDEQINAVMTGDMEKLKTLSGDSSYQSYLVISHGN |
| | SonA(Shewanella | GD (SEQ ID NO: 2) |
| | oneidensis} | |
| Type II | split borosin alpha-N- | MQETTGNAQLVVVGTGFRAIGDLTVEARACLEQA |
| | methyltransferase | DKVLCLIGDPLVTRHIEKLNASVETLDVHYAVGKP |
| | WP_031073184 | RSASYEDMVEHIMSELHRDQFVCVALYGHPGVFA |
| | StrM (Streptomyces sp. | YTGHEAIRRAREEGIAARMLPACSAEDWLFADLGL |
| | NRRL S-118) | DPGERGCQSFEATDFLIRHRVFDPTGLLILWQVGVI |
| | | GMIDRDPGYDARPGVTTLTDALVASYGSGHPVTVY |
| | | EASPYVTAEPRTTTVPLAELPDTPLSAASTLVVP |
| | | PLPPRPVDRELLARLAARR (SEQ ID NO: 3) |
| Type II | split borosin precursor | MPAAVVDFMEELVTQPRRQHAYRRSAEAYVADSA |
| | WP031073186 | LTASEREAVVSGDVDRMRAVLAEHSGVKEECHAV |
| | StrA (Streptomyces sp. | LVVIIFDPDEVPSGA (SEQ ID NO: 4) |
| | NRRL S-118) | |
| Type II | split borosin alpha-N- | MRAAPMAETETPPAAPSPSAPERPRGSLTVVGTGLR |
| | methyltransferase | ALSHMTLEAISHIRDADRVFFSVPDGVTARQIRDINP |
| | WP_083759362 | EAVDLTQYYGEDKRRKQTYVQMSEVILREVRAGS |
| | RceM (Rhodospirillum | AVTAVFYGHPGFFVFPARRILSIARKEGYRAVMLPG |
| | centenum SW) | ISSLDCLMADLRVDPSVNGCQILEATDLLLRNRPIIT |
| | | SGHVIILQVGSVGDSAFSFTAGFRHAKRAVLFERLIE |
| | | AYGEEHRSVLYLAATYPGLDGQAVVRPLGAYRDP |
| | | KVLASVPPAGTLYIPAKDMLPTDMAMAEKLGMSA |
| | | LVGPDAPVPAGPDSYGPFEAQAIAALDHYRPSPTW |
| | | RPRTASKALQRVMTLLAGTPSVAAVYRKDPARLV |
| | | DLHPDLTPAERKALLSRRAGPLNAVTAPPPEGAPPT |
| | | VDEAGNGNGGDAPSEGETA (SEQ ID NO: 5) |
| Type II | split borosin precursor | MTTIVPTELDQPDVIELSGGELDVAELSGGELDVAE |
| | WP_012568692 | LFGGELDVAELSGGELDVAELSGGELDVAELSGGE |
| | RceA (Rhodospirillum | LDVAELSGGELDVAELSGGELDVAELSGGELDVAE |
| | centenum SW) | LSGGELDVAEIGIINTFDL (SEQ ID NO: 6) |
| Type III | split borosin alpha-N- | MSEAKGRLVVIGSGIKAVSHFTLEAQAHIQQADIVL |
| | methyltransferase | YAAADPVTDMWIESQNPNAFDLYQYYADDKARLI |
| | WP_0069751303.1 | TYVQMIERIMAEVRAGKYVCALFYGHPGVFVTPSH |
| | BstM (Burkholderia | NAIAIARQEGFDAVMLPAVSAEDCLYADLGVDPSV |
| | stabilis) | PGMQIYEATDFLLRRRKVDTTANFVLWQVGCIGDL |
| | | GFKFGGYKNDKFDVLLDYLEEIYGADHPAINYVAN |
| | | MFSGPPQIDRHVIGDYRDPEVKAKVSGISTFFIPAKD |
| | | GIQSDAGMLAKLGLAKISEARRTPLICDREDYRVLA |
| | | DIARRNIHNHKVPPGYKYSFASDALYQTLLTLALD |
| | | QRAQGEFQANPRAYLEARPGLTADERRSLLLQHTG |
| | | VTRMMFKRDPDKEAVRFVGAALLDPALAREYRDR |
| | | QAAAAQAVANHDIPVGEYEGVVASWLRGQGYAA |
| | | TPAAVTRAMEEVYTSKDHLSVA (SEQ ID NO: 7) |
| Type III | split borosin precursor | MASFDVSGTYMTSWLQPDGKTYIPGPVVIDTSNQS |
| | WP_069751302.1 | VTLNGATIASPSFTTTGVSWAATGGNATSAKLTFY |
| | BstA (Burkholderia | QNGGVNGFVGLFSQGSDPLPAANNLFGNAGAATQ |
| | stabilis) | QLSTWNGTYNLFSLTADGKSTPLSDKLVISAPQVTY |
| | | GGSIVNKPVYTGEKGPKGEIDQLAWFSANGNPQNA |
| | | IIQFSNSQAPTLTLYGELWNSGAQPTYVNLSGTTKS |
| | | SSTPPTITVVAALISNTATEVTTEVTTEVTTEVTTEV |
| | | TTEVTTEVTTEVLTEVAVAVVEVLAEDDDKPSPGS |
| | | AKASLPKEDAAAAEKLLEKALALAE(SEQID |
| | | NO:8) |
| Type IV | split borosin alpha-N- | MELAHQIYADIPSLSDTAAREPATATGELTIIGSGIG |
| | methyltransferase | VMGFTRDAEQYIDDADHVVFCVADPATQVWLRGR |
| | EGP43617.1 | RPDAIDLFALYDDRKPRYHTYMQMTEAMLYHVRR |
| | AinM (Achromobacter | GKRVAAVFYGHPGIFVLSTHRAVAIARREGHRAQL |
| | insuavis) | RPGVSALDWLCADLGIDPAYPGMQTFEATDMLLR |
| | | RRAIDPGSHVVLWQVGLIAEMGFRRRGFHNRRFDY |
| | | LVEYLRGYYKPDHKIVHYIASRYPTLPPVIERYTLD |
| | | ELQNPEVHALITGISTFYVPPAQARAVDVDFAREVG |
| | | LIQPGQHVGKPRLSRPLDRYGPREMRAIAALKDFRP |
| | | PADYHFQTDTAAARFLLALSEQPELRRRFEANPEH |
| | | ALAAFPGLSAEERKQLSSRRTARTQRAARGAAVSIS |
| | | PGEQFVIDLETKPAVANQWMQLLNTAVKASPKNW |
| | | QPVEDWLSQNYQGLALADVSDAVSSTQAWLLLM |
| | | WNGVYATSGNPPQALFVMAGAPGDGAATSTVTSG |
| | | TVYLNASPLFKISFSGGQLTFSQKDGNPCNGTLTFA |
| | | NPGASTPQSVSGMLWQDSQGKPSSNNFTATLQPLP |
| | | SNPLSVWTGEYATVYTDNQQAGPGVAVFLPSTTNP |
| | | SPTLYIGGQEVSGTSFANPTITWSGGQLSFALDASTT |
| | | GALTLSGNIAGRAITGNTVASSATGFAGQYATQHV |
| | | ASVKGANVWQPFYPLGFTAPTSGSTTCTAWLGSQS |
| | | FTAQFSNRQLTWSNGPSQIPNGQLSLSVNALTNAAC |
| | | FVGVTWANGDAKPTSPNIQGIASANSPANFVGNYN |
| | | TTLDQQPAQVLSIGGDPNSIQVTYGSEKVTYSFFTG |
| | | QLSGTTSSGLKLNAVFKYVTQGSHTPAQYVTAFTG |
| | | TQTVNSAQHQWDSSQLSNDVQQWLGTYTTFLVDP |
| | | GNGSLTAGGPTLTLSGTAASFTVQITADGTTTTLQN |
| | | PKYQITGNTLYWSGENVSGGSNYNNGAISFYLDPR |
| | | KQQRAFKGVFYANKPAPNAINWYGTPAGNNPSPPS |
| | | PGFPWWGYLLIGLGIVGLGVGGLLLWRARASGYQ |
| | | RLATEDSIEMWEKKCQ (SEQ ID NO: 9) |
| Type IV | split borosin precursor | MANTRLLEGGTCQDLEDDFDEQSEEFDNASDDASL |
| | EGP43618.1 | NNPEELDIGDDVEIPDDPNVDVDVDADVEADIETEV |
| | AinA (Achromobacter | DVDVDVDVTLEMFLEEDLERSEPGIDFVTGADGLD |
| | insuavis) | GDERP (SEQ ID NO: 10) |
| Type V | split borosin alpha-N- | MSMTTRNNREHAQATHYPSAEEHRRRWRRLAQAI |
| | methyltransferase | AARAASISDDPALLVAPQRPGTLEILGSGIEASDFSR |
| | WP_051555776.1 | SDEARILVADHVFYCVADPATKIWILSQRPDAYDL |
| | PmoM (Pseudomonas | YVLYDDSKPRYLTYMQMTEAMLHHVRNGEHVVAI |
| | mosselii) | FYGHPGVFVLSTHRAVTIARREGHHASMRAAVSAL |
| | | DTLCADLGVDPSQPGMQMYEATDMLIRRRQPDPG |
| | | LHLVLWQVGLIGELGYRRQGYLNSNFAVLLDYLED |
| | | LYGPEHPVINYVGSRYPGIDPLIDRQTLASLRDPLAQ |
| | | SWVTGISTFYLPPRTAGQSDPQMLERLGLIRPGQPV |
| | | RAASDPLRVIDRYDKRERRAFSDFARFDVPASYQW |
| | | QADTGAGRFILALSDDAELRRQYRDDPQAAVQAW |
| | | GGLDARERHLLGQRDPGAVQLAAKGADAQRHPGN |
| | | REGVEHLLTYTSASSALHRALSAAAPGELRAAAAA |
| | | WSRKAGLSIDWPGMNAELNALLQSSLAPWSGFYL |
| | | DSSRRMSLSLFSRMKNAGLRVDLDGQPLVGVRYQS |
| | | GVLTWSAEAGNGSSGYLQSDLSVQGGRSWIGLVW |
| | | PAGEQAGSGYKVALRGQSLARPACLAVGDYRVAG |
| | | EALRIVPSASASQGVDVLREGVALPGEVLFNGRDT |
| | | RIGDRRLALSTRRFEDLAQWAQGAYRLRLVHGRLA |
| | | ELLALRLGPYGLEIAGKPVTAEFREGRIQWQGGPPA |
| | | VESGQLDVTLDPITLKPLLHGQGRSAAGNKVQLRG |
| | | MALIEPLWIDQLLEQPRLGLPEWAWRHLVAVMVA |
| | | ASDKGGIFLWHGYDRARGNLRLLREALARLRQDE |
| | | MEDAG (SEQ ID NO: 11) |
| Type V | split borosin precursor | MATYPVQITNNTNLALDIYTTLNKNPATDPPSTNPA |
| | WP_028693000.1 | DYTAVYTLQGSVGANQSTTLPLTESLARLVIVRQSD |
| | PmoA (Pseudomonas | QFPLLVQVANALLPDSEQVQVGNQDVTTANSGWA |
| | mosselii) | FYQSFISQPFTPTALEFSELVSETPANALNDKAASFF |
| | | AANGYPGVSFALFSALGYWANNQLYAYPGTYYCY |
| | | EPPSGNSMGFILPTTSVGTLTIAGGKANYSPSTGGST |
| | | ALQFQYGQLTSPGADDKHGFNTTGFIRDLTWEGKP |
| | | DVITWAFVGTYDGQQFIAQSYQSPQLPWYAVAYD |
| | | MAYGALFTVQLAMTLDAAINLLGTVANGMQWLA |
| | | QNTGKLISRIQDSLNSTGDTAGAGSGVGDAADPVN |
| | | VDVDIDVDIDVDVDVDIDIDIDVDVDVDIDIDVDVD |
| | | FIAVVDVDVDVDIDVDIDVVTDTETDIDIDVDVDID |
| | | TDVNVEPGALMKVVNGVGNWIMTKALPTLIEGAVI |
| | | YVAFQSVGAIFQAWKNQDEKDIENLQPRQSTGVGV |
| | | LVNYMLQDDKPVAARWQTFSQYVAEVQGDPKTV |
| | | GVTISTLLQTGNTKADNDAANWRWSSDDENQVVA |
| | | SMAPYTGDQACKAFVILGNATYQGKPLPVKVGAS |
| | | VAMKYLAAQGA (SEQ ID NO: 12) |
| Type III | split borosin alpha-N- | MKKGSLIVVGSGIKGVAHFTVEAQGWIREADVVPY |
| | methyltransferase | CVSDPVSEVWIKENSKHSIDLYQFYGNEKQRINTYN |
| | WP_022584803.1 | EMVDEILSHVRSGKDTCAVFYGHPGIFVHPSHKAISI |
| | BlaM (Brevibacillus | ARSEGYKAAMLPGISALDCLCADLGVDPSVTGMQT |
| | laterosporus PE36) | VEATDLLLRNRRLNTDQNVVIWQIGCVGDLGFNFS |
| | | GYDNRNLKILVEYLEKFYAKDHMVTHYQGSQYSIC |
| | | PPSIAKMPLSELKNAPVTGISTLYIPPQDKLKLDEEM |
| | | VQRLGLRKTQTVNTQKVAEPARKTDQQKVQPRKA |
| | | SYNQYVAAPDHSELANFLAELSESPLLLAQFMRNP |
| | | EITSSLMADLSPSEKDALLSQHPGKIRMAIKLSSWN |
| | | KHLKGVSTLIKQRENNF (SEQ ID NO: 13) |
| Type III | split borosin precursor | MNKRTENNIESYDDAQKFFQQLLINPQLAKEYVEPI |
| | WP_022584802.1 | QQAQINENPDVITTWLASKGYNTNPDEITQAQEQM |
| | BlaA (Brevibacillus | QNTDLIYWAGIYGRTSVSSDIPPNKNSVFKPGPPLV |
| | laterosporus PE36) | VQDDKTVVLNSIPLKNFTFKNKTLTWGFSDNSTAG |
| | | SITFVEVPSVNSDSTAPQSYTGKEFTGTIQMSSGEEK |
| | | EFYSGQIGPITAFPLANWSGYYGETSIEQSANNYIAG |
| | | PTLIVRDNETVLLNNQPLVNFTYDNIKNQLTWSQQ |
| | | DNDSSGSIYFARTTTPTQTGYVGLYFHGTIKQSSDQ |
| | | SLPYTGHISTPKKLEDWTGVYGQTVLTDSDNKHSQ |
| | | GPELKVISNTRVTLDGVDLKNFSYDENQKLLTWSIA |
| | | DNSTAGNITFGKITTPTSQGYVGNYFEGTLQQSKAS |
| | | EPLKYFGEVGTASSYSGGYQPSTVEKVFQILGYVAT |
| | | VAGLAQMVYVGYKVGKWAWEKLFKSQSETSEITD |
| | | GLAEGVEPELVRYTPIDPENPPPINTTSETTTETVAE |
| | | PVTTTETEVVTSEVTSEVTTEVTTEVTTEVTTEVVE |
| | | VVEVVEVVEVVEVVEVVEVVEVVEVVEVVEVVFA |
| | | EDEEKDEEKDEDEDVKKED (SEQ ID NO: 14) |
| Type III | split borosin alpha-N- | MNKNGKLIVIGSGIKSIAHFTLESQAHLQQADIVLY |
| | methyltransferase | AASDPVTDMWIQKQNPNSFDLYQYYGNTKNRIITY |
| | ADB39711.1 | TQMIERVMMELRSGKYVCALFYGHPGVFVTPSHN |
| | SliM(Spirosoma | AIELARREGYEAEMLPGISAEDCLFADLGVDPSIPGL |
| | linguale DSM74) | QTYEATDLLLRQRSINTEINAVIWQVGCVGDVGFK |
| | | FHGYDNEKLNILLDYLDKFYPPDQIVYNYVASMFS |
| | | MAKPKKDKFKLSDFRDPSIAKEVTGISTFFIPAVTM |
| | | TESDIEMSKKLGLKSGKSRSNPLICDHELYPSYKKV |
| | | ALKNISEHLIPEGYKFSHSSDALYNLVTRLALDFKE |
| | | LYKYRENPSAYLKLIPELTPTERNMLTMQHHGALR |
| | | MLFKRDRMEEARLFVDEAIKKPTIANEYLKKQQAE |
| | | YDALINEEISESDYEARLVKWFLDKGYATTPSAVTA |
| | | SINVLEMPDIIDFSGEYQCVLTMNDSKQKVSVKVNI |
| | | LDQTIAVNNELIETPYFGGDHVIWSKEDKNNSTGVL |
| | | TFSKQLNAFILEGKYANNSSVLPETYNLHGVTDTA |
| | | YN (SEQ ID NO: 15) |
| Type III | split borosin precursor | MTATNFSGNYMSFVLGVDGNTWGPGPVVAVNTTSQ |
| | ADB39710.1 | TLFIDGVQVNNPTFTSTSVMWMASSNNPSSGSIKF |
| | SliA(Spirosoma | YSSAPDTGGFIGTYVEGTAPLPSSNNFSGVSSAAPD |
| | linguale DSM74) | DLSVWNGTYNTFILNGSKWTQDSTLIVAAPNISYN |
| | | GKDISNYIYMGTKSTQNLDQLSWFVAGGNAQNAIV |
| | | EFFKDSSGYLTFGGTQWVSGDAPAANNFIGTTKAT |
| | | PEPPTITAVVAIVINSSTTEVAEVTEVTEVTEVTE |
| | | VTEVVEVVEVVEVVEVIAAAEEAEVKGQQQQSKIS |
| | | TAQQDQEAAYKLSRKSNG (SEQ ID NO: 16) |

Nucleotide sequences and amino acid sequences encoding split borosin alpha-N-methyltransferases and split borosin precursor peptides can be derived from any species, provided that the split borosin alpha-N-methyltransferase domain is less than 70% similar at the amino acid level to the canonical borosin OphMA domain and the split borosin precursor peptide domain is encoded by a separate gene that does not encode the split borosin alpha-N-methyltransferase. In some cases, nucleotide sequences and amino acid sequences encoding the split borosin alpha-N-methyltransferases and split borosin precursor peptides are derived from one or more of the following species: Shewanella oneidensis MR-1, Streptomyces sp. NRRL S-118, Rhodospirillum centenum SW, Burkholderia stabilis, Achromobacter insuavis AXXA, Pseudomonas mosselii ATCC BAA-99, Brevibacillus laterosporus PE36, and Spirosoma linguale DSM74. Split borosin gene products from these species vary in size from 250-1100 amino acid N-methyltransferases and 70-700 amino acid borosin precursors.

In some cases, the split borosin alpha-N-methyltransferase comprises an amino acid sequence having at least 70% (e.g., at least 70%, 75%, 80%, 85%, 90%, 95%, 98%, 99%) sequence similarity to an amino acid sequence selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15. Exemplary amino acid sequences of split borosin alpha-N-methyltransferase of this disclosure include, without limitation, SEQ ID NO:1, SEQ ID NO:3, SEQ ID NO:5, SEQ ID NO:7, SEQ ID NO:9, SEQ ID NO:11, SEQ ID NO:13, and SEQ ID NO:15.

The term “target peptide” is used herein to refer to any peptide that can serve as an alpha-N-methylation substrate for a split borosin alpha-N-methyltransferase. Preferably, the target peptide used with the present invention is a split borosin precursor peptide. The precursor peptide can encode one or more core peptides that can be N-methylated as described herein. Any appropriate split borosin precursor peptide can be used in connection with the methods of this disclosure. In some cases, the target peptide is a split borosin precursor comprising an amino acid sequence having at least 70% sequence similarity to an amino acid sequence selected from SEQ ID NO:2, SEQ ID NO:4, SEQ ID NO:6, SEQ ID NO:8, SEQ ID NO:10, SEQ ID NO:12, SEQ ID NO:14, and SEQ ID NO:16. Exemplary amino acid sequences of split borosin precursor peptide domains of this disclosure include, without limitation, SEQ ID NO:2, SEQ ID NO:4, SEQ ID NO:6, SEQ ID NO:8, SEQ ID NO:10, SEQ ID NO:12, SEQ ID NO:14, and SEQ ID NO:16.

In some cases, the split borosin alpha-N-methyltransferase acts on multiple alpha-N-methylation substrates. For example, the methods can comprise contacting a split borosin alpha-N-methyltransferase to a single precursor peptide substrate or multiple precursor peptide substrates whereby a library of alpha-N-methylated polypeptides is produced, either in vivo or in vitro.

As used herein, the term “encoding” refers to the inherent ability of specific sequences of nucleotides (e.g., a gene, a cDNA, or an mRNA) to serve as a template for the synthesis of a defined sequence of nucleotides (i.e., rRNA, tRNA and mRNA) or a defined sequence of amino acids. For example, a gene encodes a protein that may be produced if the gene is transcribed into mRNA that is then translated into a protein. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. As used herein, a nucleotide sequence that encodes a protein or an RNA may also include introns to the extent that the nucleotide sequence encoding the protein may in some version contain an intron(s).

As used herein, the terms “identity” and “sequence identity” refer to the subunit sequence identity between two polymeric molecules, particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the term “sequence similarity” refers to percent similarity of two sequences and is determined by the sum of identical and similar matches. Like sequence identity, sequence similarity is expressed as a percent and is computed by considering all identical and similar matches. A match is “similar” if there is a conservative substitution whereby physiochemical properties are preserved. The similarity between two proteins is determined using pairwise alignments and depends on the criteria of how two amino acid residues relate to each other. By way of example, a change from arginine to lysine maintains the +1 positive charge and is considered to be a conservative substitution. Such a substitution is more likely to be acceptable since the two residues have similar properties. Most similarity scoring matrices give higher scores for conservative substitutions even if amino acid/base is changed, resulting in non-identical residues at a particular position. Substitutions that result in a functional change are not considered to be “similar,” for example, replacement of a basic amino acid with an acidic amino acid. In some cases, sequence similarity is calculated with a BLOSUM62 matrix using a software program such as Geneious.

Evaluating the structural and functional homology of two or more polypeptides generally includes determining the percent identity of their amino acid sequences to each other. Sequence identity between two or more amino acid sequences is determined by conventional methods. See, for example, Altschul et al., (1997), Nucleic Acids Research, 25(17):3389-3402; and Henikoff and Henikoff (1982), Proc. Natl. Acad. Sci. USA, 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the shorter sequence plus the number of gaps introduced into the longer sequence in order to align the two sequences])×(100). Those skilled in the art will appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of another peptide. The FASTA algorithm is described by Pearson and Lipman (1988), Proc. Nat'l Acad. Sci. USA, 85:2444, and by Pearson (1990), Meth. Enzymol., 183:63.

As illustrated in the Examples that follow, the methods can be in vitro (i.e., outside of a living organism) or in vivo (i.e., within a living organism). For in vitro methods, it is preferable for the split borosin alpha-N-methyltransferase and target peptide to be isolated proteins. In such cases, methods of this disclosure comprise expressing a split borosin alpha-N-methyltransferase polypeptide in vitro and contacting the expressed polypeptide to a target peptide for which alpha-N-methylation is desired. In some embodiments, one or more of the split borosin alpha-N-methyltransferase and the target peptide are expressed as recombinant proteins, i.e., proteins made by artificially combining two or more otherwise separated protein segments. In some embodiments, one or more of the split borosin alpha-N-methyltransferase and the target peptide are synthetic proteins, i.e., proteins that are produced using chemical protein synthesis outside of a living cell.

Any means of obtaining isolated proteins can be used. For example, isolated proteins can be obtained by recombinant or synthetic methods known in the art. In some cases, an isolated split borosin methyltransferase protein is obtained by: introducing into a cell a nucleotide sequence encoding a split borosin methyltransferase protein, where the nucleotide sequence is introduced in an expression vector; expressing the split borosin methyltransferase protein in the cell; and purifying the expressed split borosin methyltransferase protein.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. A protein, peptide, or polypeptide may be just a fragment of a naturally occurring protein or peptide. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex.

In some cases, peptides, polypeptides, nucleic acids, and other biomolecules of this disclosure may be isolated. As used herein, “isolated” means to separate from at least some of the components with which it is usually associated, whether it is derived from a naturally occurring source or made synthetically, in whole or in part. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Peptides, polypeptides, nucleic acids, and other biomolecules of the disclosure may be purified. As used herein, “purified” means separate from the majority of other compounds or entities. A compound or moiety may be partially purified or substantially purified. Purity may be denoted by weight measure and may be determined using a variety of analytical techniques such as but not limited to mass spectrometry, HPLC, etc.

In some embodiments, recombinant expression of a split borosin protein of this disclosure in a host cell is achieved by introducing an expression vector comprising a nucleotide sequence encoding the split borosin protein of interest into a host cell. Any appropriate nucleic acid vector can be used with the methods provided herein. The term “expression vector” or “vector” as used herein refers to a recombinant DNA molecule containing a desired coding sequence and appropriate heterologous nucleic acid sequences necessary for the expression (i.e., transcription and/or translation) of the operably linked coding sequence, for example, heterologous promoter sequences. The heterologous sequence (i.e., sequence from a difference species than the coding sequence) can comprise a heterologous promoter or heterologous transcriptional regulatory region that allows for expression of the polypeptide. As used herein, the terms “heterologous promoter,” “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Expression vectors include all those known in the art including, without limitation, a yeast artificial chromosome, bacterial plasmid (e.g., naked or contained in liposomes), phagemid, shuttle vector, cosmid, virus (e.g., Sendai viruses, lentiviruses, retroviruses, adenoviruses, and adeno-associated viruses), chromosome, mitochondrial DNA, plastid DNA, and nucleic acid fragment. Generally, an expression vector comprises sufficient cis-acting elements for expression; other elements for expression can be supplied by the host cell or in an in vitro expression system.

For in vivo methods, contacting of the split borosin alpha-N-methyltransferase to the target peptide occurs in a host cell. For example, the method can comprise expressing in vivo a split borosin alpha-N-methyltransferase polypeptide in a host cell. In some cases, the split borosin alpha-N-methyltransferase is co-expressed with a target peptide for which alpha-N-methylation is desired. In such cases, the method comprises introducing into a cell one or more vectors (e.g., plasmids) encoding the split borosin methyltransferase protein and the target peptide. In one embodiment, the split borosin alpha-N-methyltransferase is encoded in a first vector and the target peptide is encoded in a second vector. In another embodiment, a single vector encodes both the split borosin alpha-N-methyltransferase and the target peptide. Any appropriate method of introducing nucleic acid sequences or vectors into a host cell can be used. In some cases, nucleic acids are transfected into a non-human host cell. The term “transfected” or “transformed” or “transduced” as used herein refers to a process by which exogenous nucleic acid (including, e.g., cDNA and vectors) is transferred or introduced into the host cell (e.g., a prokaryotic cell, a eukaryotic cell). A “transfected” or “transformed” or “transduced” cell is one which has been transfected, transformed or transduced with exogenous nucleic acid. The cell includes the primary subject cell and its progeny.

In some cases, a split borosin alpha-N-methyltransferase and/or a target peptide (e.g., a split borosin precursor) comprises an affinity tag and/or a solubility tag. As used herein, the term “affinity tag” refers to a member of a binding pair, i.e. a pair of two molecules wherein one of the molecules specifically binds to the other molecule through chemical or physical means. Affinity tags suitable for these methods are readily known in the art and include, without limitation, a histidine tag, FLAG tag, glutathione transferase (GST) tag, Halo tag, Streptavidin binding peptide tag (Strep-II), Calmodulin-binding protein tag (CBP), Staphylococcal Protein A tag (Protein A), Intein mediated purification with the chitin-binding domain tag (IMPACT), Cellulose-binding module tag (CBM), Dockerin domain of Clostridium josui tag (Dock), fungal avidin-like protein tag (Tamavidin), Albumin-binding protein tag (ABP), Biotin-carboxy carrier protein tag (B-tag), Choline-binding domain tag (CBD), Human influenza hemagglutinin tag (HA), polyarginine tag (Arg-tag), polyaspartate tag (Asp-tag), polycysteine tag (Cys-tag), polyphenylalanine tag (Phe-tag), and Universal HTTPHH tag, etc.

As used herein, the term “solubility tag” refers to moiety that is added to a peptide to enhance its solubility. Exemplary solubility tags include, without limitation, SUMO tags, maltose-binding tags (MBT), Fasciola hepatica 8-kDa antigen tags (Fh8), N-utilization substance tags (NusA), Thioredoxin tag (Trx), solubility-enhancer peptide sequence tags (SET), IgG domain of Protein G tag (GB1), IgG repeat domain ZZ of Protein A tags (ZZ), Solubility eNhancing Ubiquitous Tags (SNUT), Seventeen kilodalton protein tags (Skp), Phage T7 protein kinase tags (T7PK), E. coli secreted protein A tags (EspA), E. coli trypsin inhibitor tags (Ecotin), Calcium-binding protein tags (CaBP), Stress-responsive arsenate reductase tags (ArsC), RNA polymerase alpha-subunit tags (RpoA), Aggregation-resistant protein tags (SlyD), RNA polymerase sigma factor tags (RpoS), Spermidine/putrescine-binding periplasmic protein tags (PotD), Acidic protein tags (msyB), Disulfide isomerase I tags (DsbA), Superfolder green fluorescent protein tags (sfGFP), Small metal-binding protein tags (SmbP), Tetracopeptide domain-containing thioredoxin tags (TDX), polycationic tags, polyanionic tags, and Dihydrofolate reductase tag (DHFR), etc.

Any suitable methyl donor may be used with the present invention. As used herein, the term “methyl donor” refers to any substrate that can be used as a source of methyl groups by a methyltransferase. In some cases, the methyl donor is S-Adenosyl-methionine (SAM) or an enzymatically active analog thereof. For example, synthetic or semisynthetic SAM analogs could be used as methyl donors. Exemplary SAM analogs include, without limitation, those described by Thomsen et al., Org. Biomol. Chem., 2013, 11, 7606-7610

For in vitro methods, conditions that are conducive for alpha-N-methylation of the target peptide should be utilized. Such conditions and concentrations are readily known or ascertainable by one skilled in the art. For example, general in vitro methyltransferase conditions are described in Lee et al., J. Biol. Chem., 2012, 287(2), 1426-1434, but suitable conditions are not limited thereto.

Split borosin alpha-N-methyltransferases of this disclosure can be used in connection with a split borosin precursor peptide domain alone or in conjunction with one or more other methyltransferase domains or modifying enzymes. As described in the Examples that follow, the methods provided herein permit one to prepare a peptide library comprising random alpha-N-methylated peptides.

For example, a split borosin alpha-N-methyltransferase can be expressed with one or more split borosin precursors that contains one or more genetic variations relative to a nucleotide sequence encoding a wild-type split borosin precursor. As used herein, the term “genetic variation” refers to a difference in the nucleotide sequence relative to the wild-type sequence. Suitable genetic variations include, for example, base-pair substitutions, insertions, and deletions. Genetic variations can be naturally occurring or can be introduced by non-natural means such as genetic engineering. Suitable means of genetic engineering a protein are known in the art and are well understood by one skilled in the art.

In some embodiments, one or more split borosin precursors comprise genetic variations produced by random mutagenesis. The term “random mutagenesis” refers to methods of randomly introducing mutations into a gene sequence. Random mutagenesis can be used to create libraries comprising thousands of variations of a gene. Suitable random mutagenesis methods include, for example, those that utilize error-prone PCR, rolling circle-error-prone PCR, mutator strains, transposon insertion, ethyl methanesulfonate, nitrous acid, and DNA shuffling. Error prone PCR methods can be divided into (a) methods that reduce the fidelity of the polymerase by unbalancing nucleotides concentrations and/or adding of chemical compounds such as manganese chloride (see, e.g., Lin-Goerke et al., (1997), Biotechniques, 23:409-412), (b) methods that employ nucleotide analogs (see, e.g., U.S. Pat. No. 6,153,745), (c) methods that utilize ‘ mutagenic’ polymerases (see, e.g., Cline, J. and Hogrefe, H. H. (2000), Strategies (Stratagene Newsletter), 13:157-161 and (d) combined methods (see, e.g., Xu et al., (1999), Biotechniques, 27:1102-1108. Other PCR-based mutagenesis methods include those, e.g., described by Osuna et al., (2004), Nucleic Acids Res., 32(17): e136 and Wong et al., (2004), Nucleic Acids Res., 10; 32(3):e26), and others known in the art.

In some embodiments, one or more split borosin precursors comprise genetic variations produced by site-directed mutagenesis. In contrast to random mutagenesis, the term “site-directed mutagenesis” refers to methods by which intentional changes are made to a gene sequence. Suitable site-directed methods include, for example, those that utilize traditional PCR, inverse PCR, primer extension, and CRISPR-based genome editing.

In some cases, the method comprises introducing into a cell one or more expression vectors comprising a nucleotide sequence encoding a split borosin alpha-N-methyltransferase and one or more nucleotide sequences encoding one or more split borosin precursors comprising one or more genetic variations relative to a nucleotide sequence encoding a wild-type split borosin precursor; and isolating the alpha-N-methylated peptides to produce the peptide library. In some embodiments, the method comprises the step of detecting production of alpha-N-methylated peptides prior to isolating the alpha-N-methylated peptides.

The split borosin alpha-N-methyltransferase/precursor pairs of the present invention are distinguished from the canonical borosin pairs in that the alpha-N-methyltransferase can methylate the precursor polypeptide when these components are expressed as separate polypeptides (i.e., in trans), allowing for a single N-methyltransferase to be able to methylate more precursor peptides. However, in some embodiments, for use with the present invention, the split borosin alpha-N-methyltransferase may be expressed as either a separate polypeptide (i.e., in trans) or as a recombinant fusion polypeptide (i.e., in cis) with the split borosin precursor polypeptide. As used herein, the term “in cis” when used in reference to an interaction of two or more entities (i.e., a split borosin alpha-N-methyltransferase and one or more split borosin core peptides) means that the two or more entities are expressed as a single polypeptide. In contrast, the term “in trans” means that the two or more entities are expressed as separate polypeptides. Advantageously, the split borosin alpha-N-methyltransferase and split borosin precursor(s) polypeptides of the present invention are expressed in trans, as the inventors have discovered that such systems are capable of multiple substrate turnover. Thus, this trans system allows for increased production of methylated target sequences with less methyltransferase present in the system.

The alpha-N-methylated peptides produced by the methods may be detected using any known methods of protein detection and protein mass spectrometry. In some cases, alpha-N-methylation is detected on the peptides, e.g., using mass spectrometry.

The alpha-N-methylated peptides produced by the methods may be isolated using any protein purification methods known in the art. Suitable methods include affinity chromatography (e.g., nickel column purification using a His-tagged protein), size exclusion chromatography, ion exchange chromatography, and HPLC.

In another aspect, provided herein are methods for enzymatically introducing selective α-N-methylations into ribosomally produced therapeutic peptides. As described herein, these methods avoid chemical synthesis steps that produce low yields, use harsh conditions, and introduce off-target modifications. Alpha-N-methylations are important chemical moieties that improve therapeutic peptide metabolic stability, membrane permeability, target selectivity, affinity, and oral bioavailability.

In another aspect, provided herein is a system for engineering precursor peptide sequences for production of selectively, differentially alpha-N-methylated peptides. Current production of alpha-N-methylated peptides are carried out by non-ribosomal peptide synthetases (NRPSs) or by chemical synthesis using pre-methylated amino acid building blocks. Both of these methods require the methylation to occur before the peptide bond is formed. As described herein, the systems of this disclosure are preferable to conventional systems because the methylation occurs after peptide bond formation. This difference makes engineering and synthesizing alpha-N-methylated peptides simpler by requiring fewer chemical steps.

In another aspect, provided herein is a vector comprising a nucleotide sequence encoding a split borosin alpha-N-methyltransferase domain. In some cases, the nucleotide sequence encodes a split borosin alpha-N-methyltransferase domain comprising an amino acid sequence having at least 70% sequence similarity to an amino acid sequence selected from SEQ ID NO:1, SEQ ID NO:3, SEQ ID NO:5, SEQ ID NO:7, SEQ ID NO:9, SEQ ID NO:11 SEQ ID NO:13, and SEQ ID NO:15. In some cases, the nucleotide sequence encodes a split borosin alpha-N-methyltransferase domain comprising an amino acid selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15.

The vector can be provided in a host cell, where the vector is introduced into the host cell by any appropriate means, including those described herein. The host cell can be a eukaryotic cell (e.g., a mammalian cell) or a prokaryotic cell (e.g., bacteria).

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

The terms “comprising”, “comprises” and “comprised of” as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

It should also be understood that, unless clearly indicated to the contrary, in any methods claimed herein that include more than one step or act, the order of the steps or acts of the method is not necessarily limited to the order in which the steps or acts of the method are recited.

The present invention has been described in terms of one or more preferred embodiments, and it should be appreciated that many equivalents, alternatives, variations, and modifications, aside from those expressly stated, are possible and within the scope of the invention. The invention will be more fully understood upon consideration of the following non-limiting Examples. The invention will be described in greater detail by way of specific examples. The following examples are offered for illustrative purposes, and are not intended to limit the invention in any manner.

### EXAMPLES

The Examples in the following section demonstrate in vitro and in vivo alpha-N-methylation reactions by split borosin alpha-N-methyltransferase and split borosin precursor domains.

### Example 1: N-Methylation of Target Peptide Using Alpha-N-Methyltransferase Described

In this Example, the inventors have demonstrated the expression and production of the borosin alpha-N-methyltransferase and split borosin precursor domains. These proteins and peptides are used in N methylation reactions to demonstrate the methylation of the target peptides. Mass spectrometry was performed to demonstrate the methylation pattern of the target peptides using the different split borosin alpha-N-methyltransferases. The ability to N-methylate in trans as demonstrated will allow for the production of peptides with favorable characteristics, including increased proteolytic stability and membrane permeability.

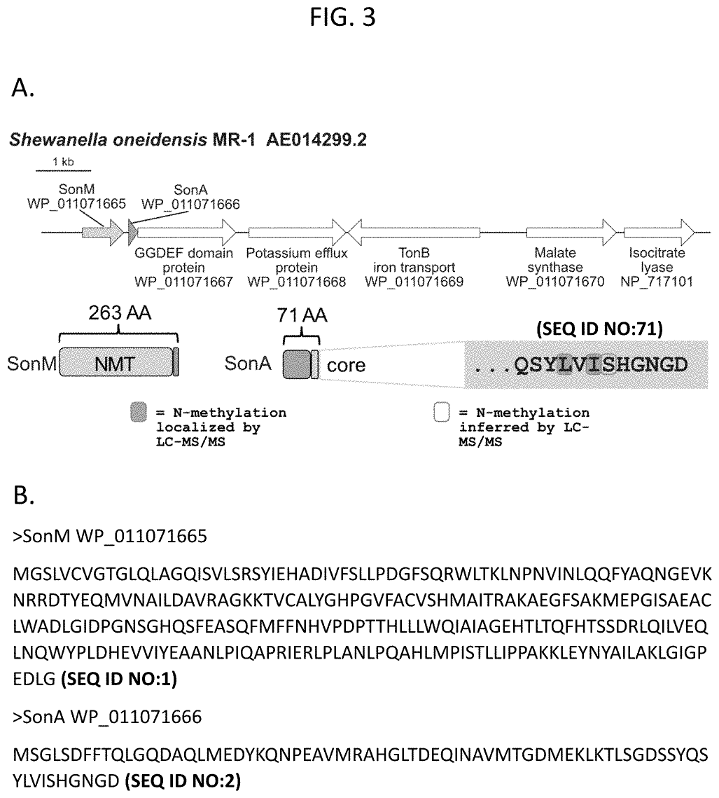

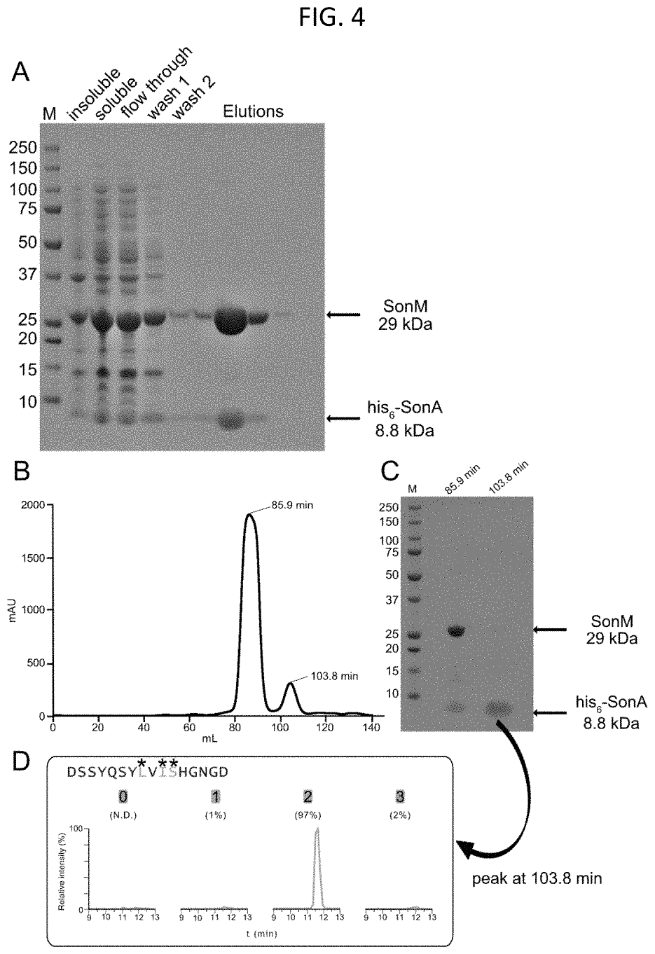

Methods for Data Described in FIGS. 3 and 4.

To make the sonM-sonA co-expression construct, pET28b backbone was digested with NcoI-HF and SalI-HF, treated with Antarctic phosphatase, and the band was extracted from an agarose gel using a kit (Thermo Scientific). The native RBS was used in the co-expression construct and an N-terminal hexa-histidine (his6) tag was added to sonA. Gene sonM was amplified using primers prmMRJ036_fw (5′-ACTTTAAGAAGGAGATATACCATGGGATCACTCGTCTGTG-3′; SEQ ID NO:43) and prmMRJ043_rev (5′-GATGATGATGATGATGCATGTTTTCTCCTTATTGTTAATAATGATTCAATAAC-3′; SEQ ID NO:44). sonA was amplified with an N-terminal his6 tag using primers prmMRJ044_fw (5′-AGGAGAAAACATGCATCATCATCATCATCACATGTCTGGATTATCGGATTTTTTT AC-3′; SEQ ID NO:45) and pRMMRJ045_rev (5′-CGAGTGCGGCCGCAAGCTTGTCGACTTAATCACCATTACCATGTG-3′; SEQ ID NO:46) in a PCR reaction. Overlap extension PCR was used to join the sonM and his6-sonA amplicons. Resulting band was excised from an agarose gel before assembly into the backbone. Assembly was transformed into electrocompetent TOP10 E. coli cells and colonies were screened via colony PCR using primers T7_fw (5′-TAATACGACTCACTATAGGG-3′; SEQ ID NO:47) and T7_rv (5′-GCTAGTTATTGCTCAGCGG-3′; SEQ ID NO:48) and OneTaq polymerase. Colonies showing a correctly sized band were sequence verified by ACGT.

Heterologous expressions were conducted in E. coli cells BL21(DE3). Saturated overnight culture (10 mL) in LB with 50 μg/mL kanamycin was used to inoculate 1 L of TB with 50 μg/mL kanamycin in a 2.5 L baffled Ultra Yield flask (Thomson Scientific). The 1 L culture was incubated in a 37° C. shaker until the OD₆₀₀ reached approximately 0.7, at which time the culture was cold shocked in an ice bath for 30-60 min. After cold shocking, the culture was induced with 200 mM IPTG and placed in a 16° C. shaker for 24 hrs. After 24 hrs, the cells were harvested by centrifugation at 4000×g for 30 min at 4° C., snap frozen in liquid nitrogen, and stored at −80° C. until use.

For protein purification by nickel affinity chromatography, frozen cells were thawed on ice and then resuspended to homogeneity in ice-cold lysis buffer (300 mM NaCl, 50 mM sodium phosphate, 20 mM imidazole, 10% glycerol, pH 8.0) with 4 mL of buffer for every 1 g of wet cell mass. After resuspension, lysozyme was then added to a final concentration of 1 mg/mL and incubated on ice for 30 min. After lysozyme treatment, cells were further lysed by sonication. After sonication, lysate was clarified by centrifugation at 15,000×g for 45 min at 4° C. The soluble protein from the clarified supernatant was then batch-bound to nickel-NTA resin (GoldBio) for 60 min on a rotator at 4° C. After binding, resin was added to a 5 mL fritted column, washed with 10 column volumes of lysis buffer, and the protein was eluted in lysis buffer with 250 mM imidazole. For subsequent gel filtration chromatography, protein was concentrated, sterile filtered and loaded onto a HiLoad 16/600 Superdex 200 pg size exclusion column was used at a flow rate of 1 ml/min of lysis buffer without imidazole. Protein was analyzed by SDS-PAGE gel, fractions were pooled and concentrated using Amicon Ultra centrifugal filter columns (MilliporeSigma). Concentrations were measured by Bradford assay and proteins were snap frozen in liquid nitrogen and stored at −80° C. until use. When using frozen protein, all samples were thawed on ice, centrifuged at top speed in a microcentrifuge at 4° C. for 10 min, aggregate removed by transferring supernatant to a fresh tube, and the concentration re-measured.

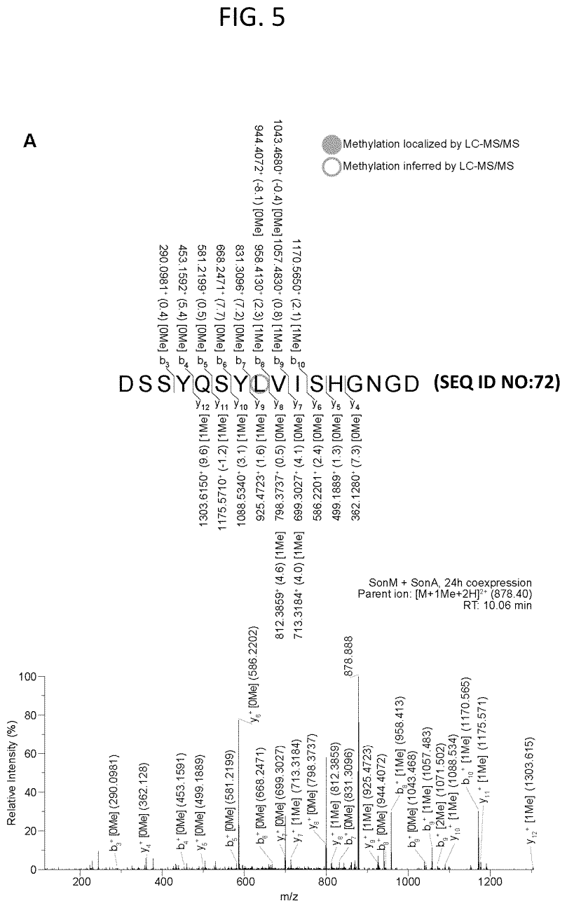

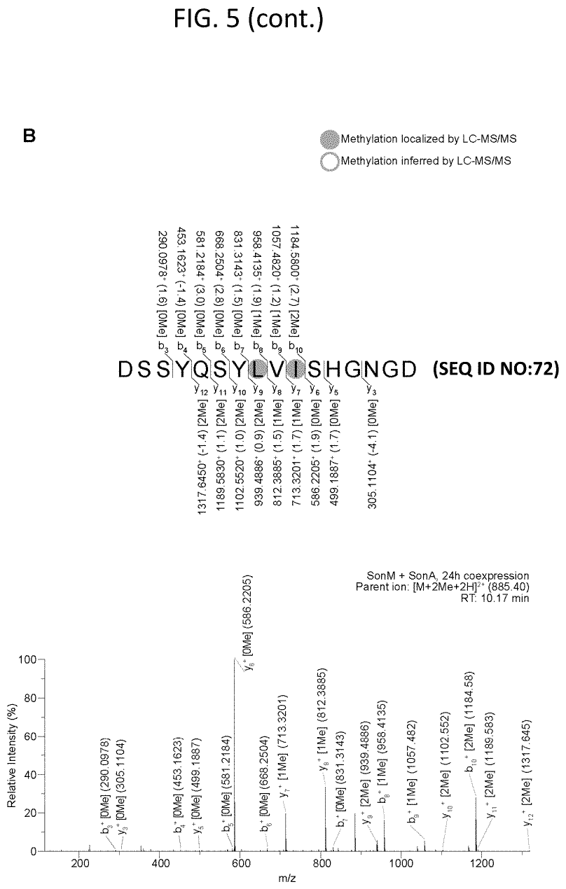

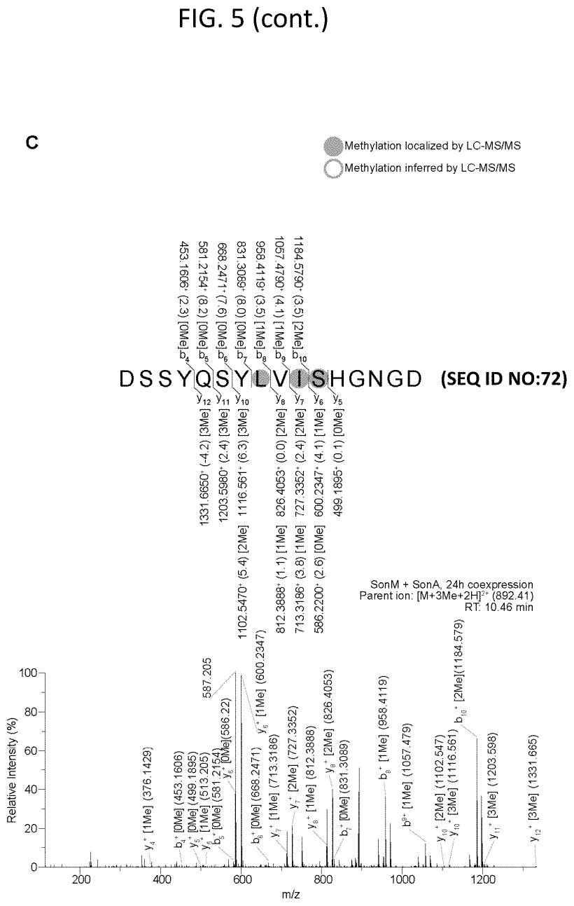

Methods for Data Described in FIG. 5.

Briefly, the band corresponding to his6-SonA was extracted from an SDS-PAGE gel, cut into ˜2 mm×2 mm pieces and placed in 1.5 mL LoBind tubes (Eppendorf). Gel cubes were then washed with a 1:1 ratio of 100 mM ammonium bicarbonate (ABC):acetonitrile (ACN) three times until gel pieces appeared clear. After dye removal, they were then dehydrated in 100% ACN until semi-opaque (˜30 sec), and the ACN was subsequently discarded. After rehydration in digest buffer (50 mM ABC and 1:50 units AspN protease (Promega)), gel pieces were placed on ice for 15 min and then were transferred to a 37° C. incubator overnight. The next day, excess liquid from the digest was collected and transferred to a new LoBind tube. Digested peptides were extracted from the gel pieces by first covering them with 60 μL of 50% ACN and 0.3% formic acid (FA) and incubating at room temperature for 15 min. After this incubation, the supernatant was recovered. This extraction was repeated with 604 of 80% ACN and 0.3% FA and the supernatant was recovered and placed into the same LoBind tube. The pooled peptide extractions were frozen at −80° C. for 30 min to deactivate the protease. After freezing, the extracted peptides were thawed and dried using a SpeedVac (Eppendorf). Dried peptides were reconstituted in 0.1% FA and purified/desalted using C18 ZipTips according to the manufacturer's instructions. Purified and desalted peptides were again dried using the SpeedVac and then reconstituted in 15-30 μl of 20% ACN, 0.1% FA, and transferred to glass vials for MS analysis. Peptide mass spectrometric analysis (LC-MS/MS HCD) LC-MS/MS measurements of digested peptides was performed as previously described.¹⁶ Briefly, data were obtained on a Thermo Scientific Fusion mass spectrometer furnished with a Dionex Ultimate 3000 UHPLC system with a nLC column (200 mm×75 μm) packed with Vydac 5-μm particles of 300 Å pore size (Hichrom Limited). Elutions used a linear gradient consisting of 0.1% FA in water (solvent A) and 0.1% FA in ACN (solvent B) at a flow rate of 0.3 μl/min. The column was initially equilibrated with 20% solvent B for 5 min and then subjected to a linear increase of solvent B to 85% over 32 min followed by a final elution step of 85% solvent B for 2 min. Mass spectra were acquired in positive-ion mode. Full MS was done at a resolution of 60,000 [automatic gain control (AGC) target, 4×10⁵; maximum ion trap (IT), 50 ms; range, 300 to 1800 m/z], and data-dependent and targeted MS/MS were both performed at a resolution of 15,000 (AGC target, 5×10⁵; maximum IT, 500 ms; isolation window, 2.2) using higher-energy collisional dissociation (HCD). HCD collision energies from 14-20% with steps of ±4% were used during LC-MS/MS measurements.

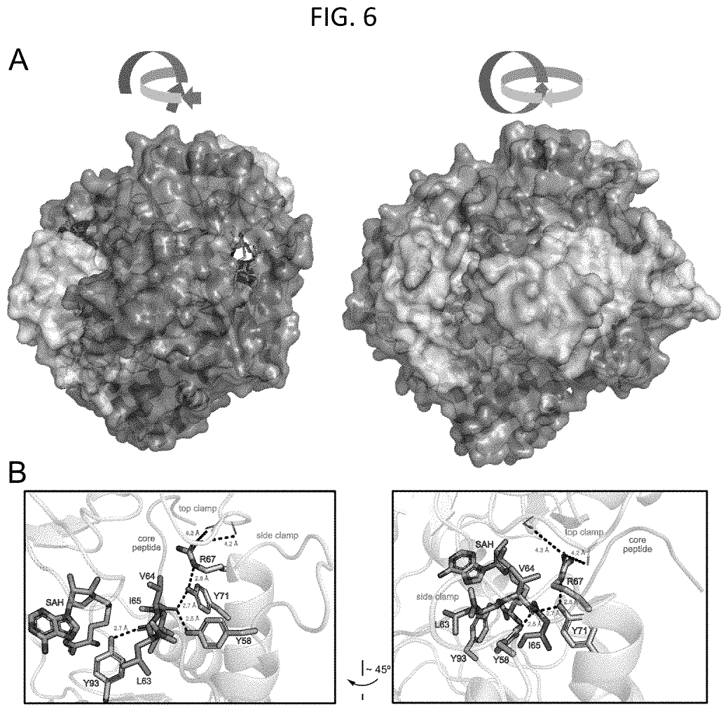

Methods for Data Described in FIG. 6.

Purified SonA-SonM protein complex was concentrated to 20 mg/mL and buffer-exchanged into 10 mM HEPES pH 8 buffer. The hanging drop method was used with 1 mL of well solution in 24 well plates. For structures with SAH co-crystallized, SAH was added to the protein solution to a final concentration of 5 mM before tray was laid. Crystal trays were kept at 20° C. and crystals formed in less than 48 hrs. Crystals were looped, treated with a cryoprotectant consisting of 20% PEG 3350, 20% glycerol, and 240 mM sodium malonate at the same pH as the well solution for that particular crystal, and frozen in liquid nitrogen for storage. XRD data sets were collected at the Advanced Photon Source (APS, Argonne, Ill., USA). Molecular replacement was performed using the structure of OphMA (PDB: 5N0Q).

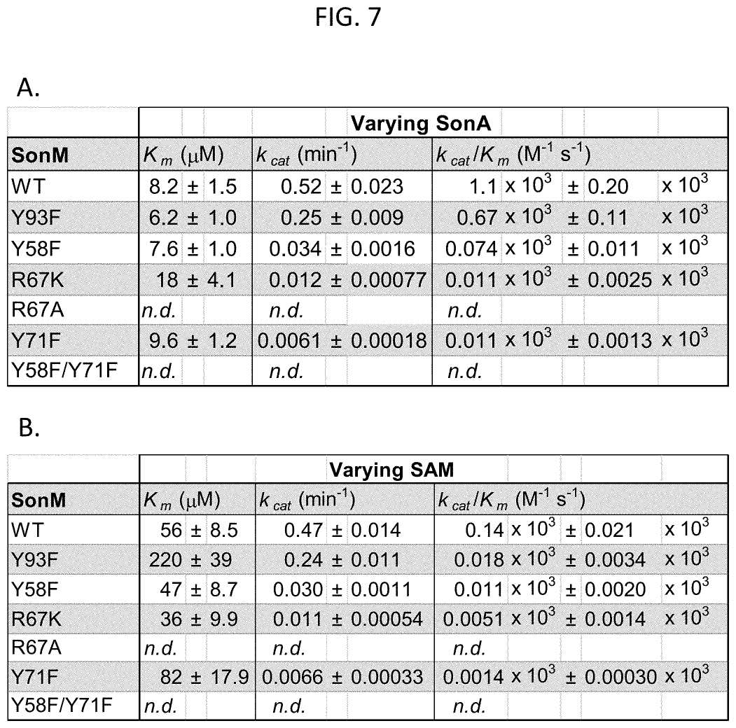

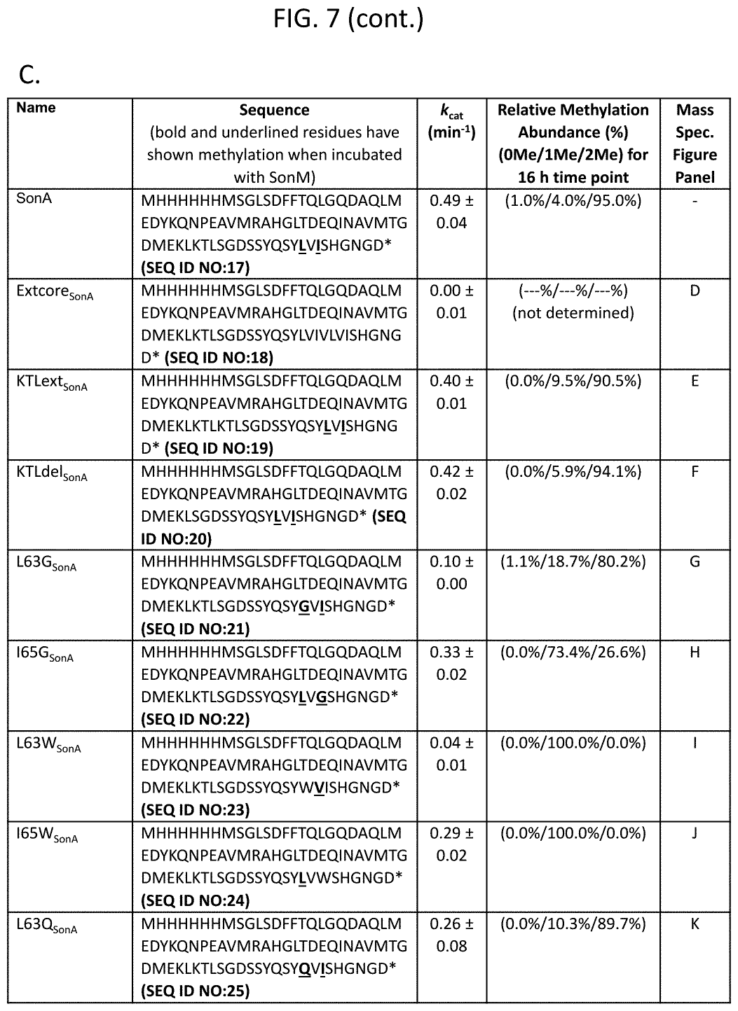

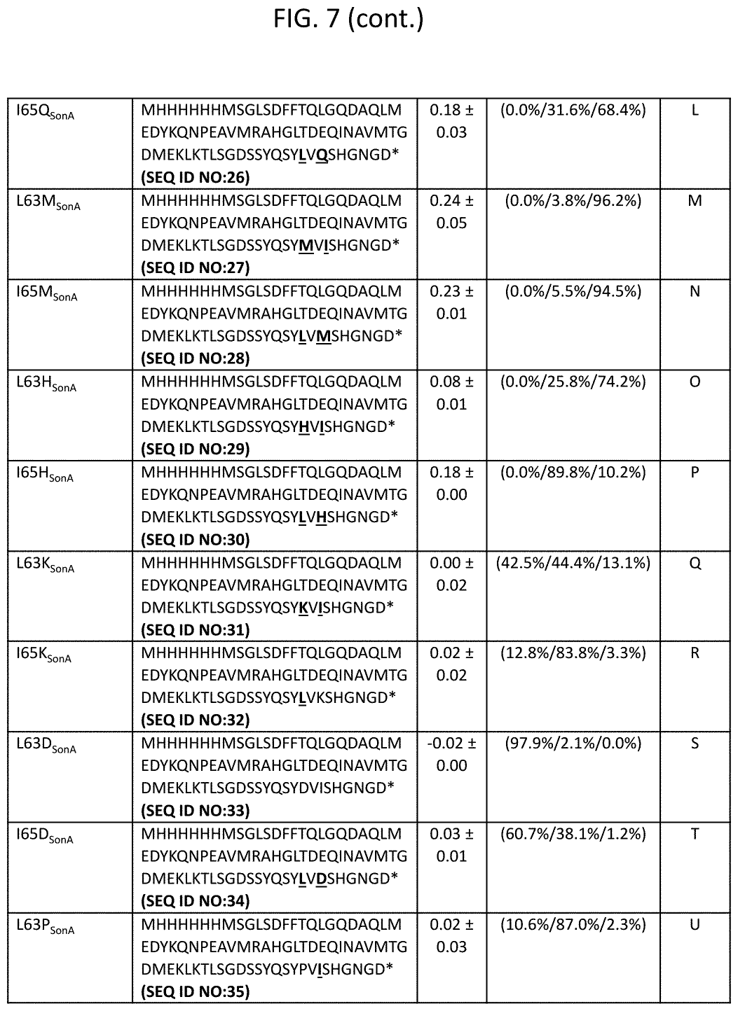

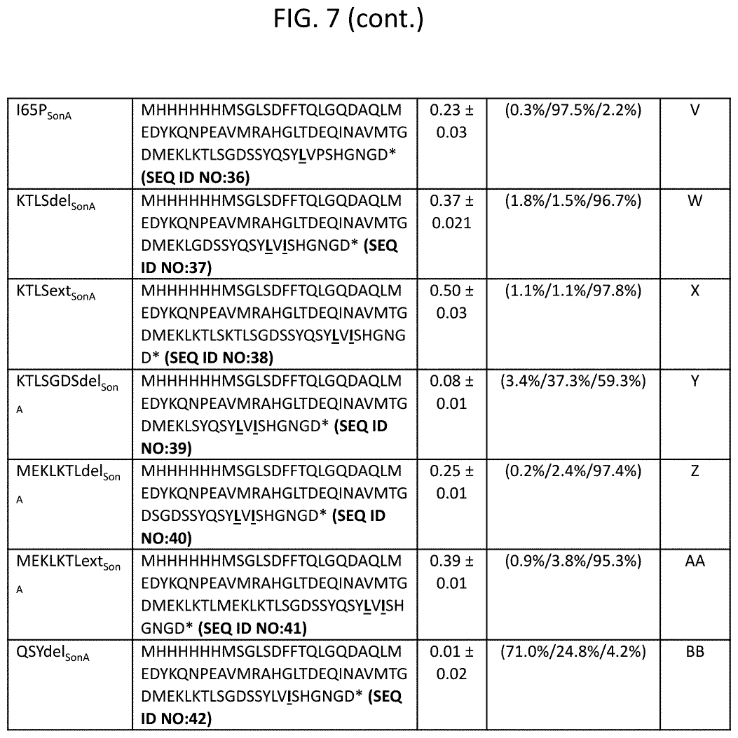

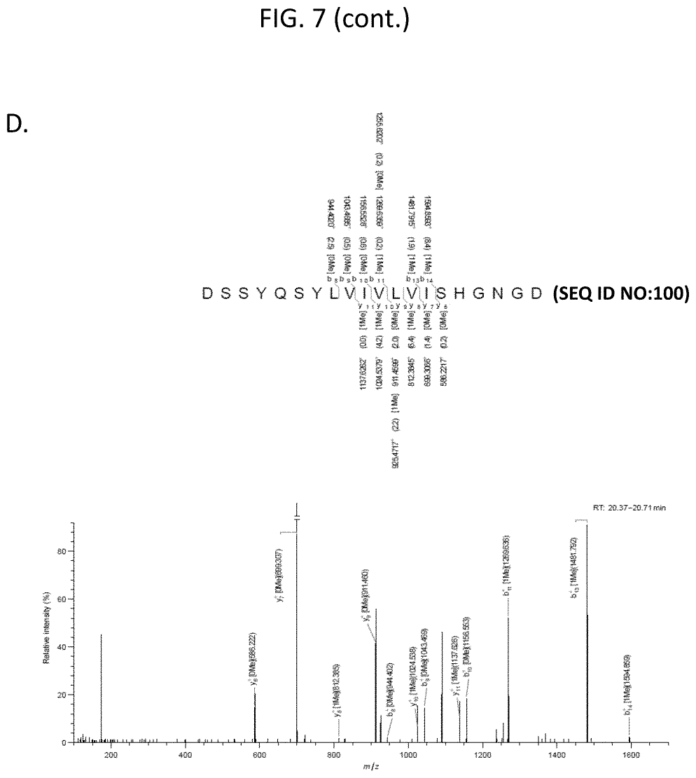

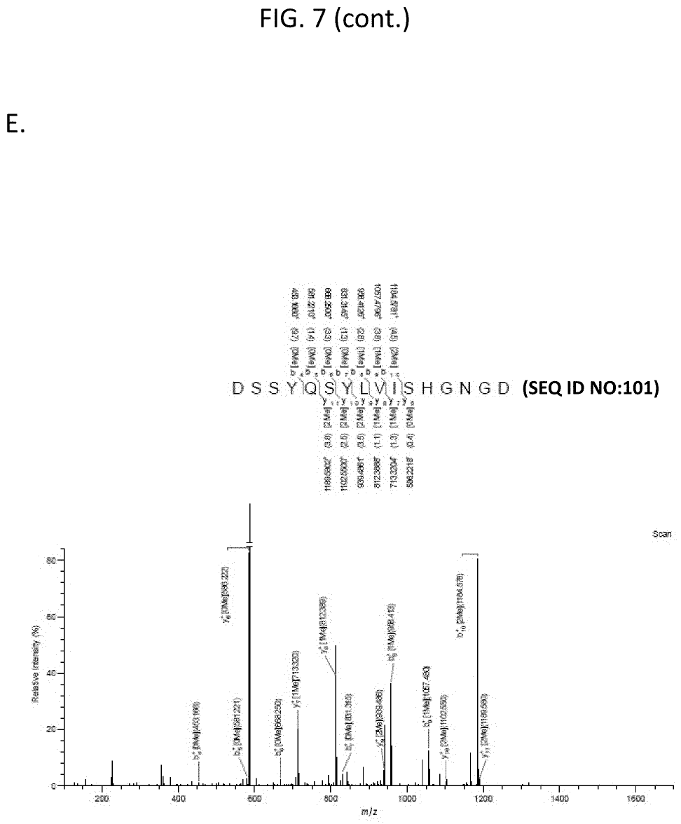

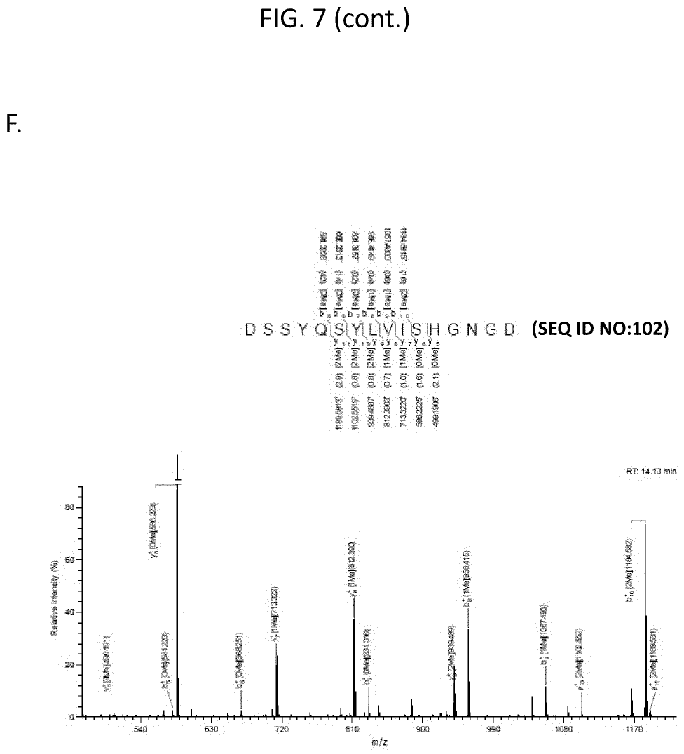

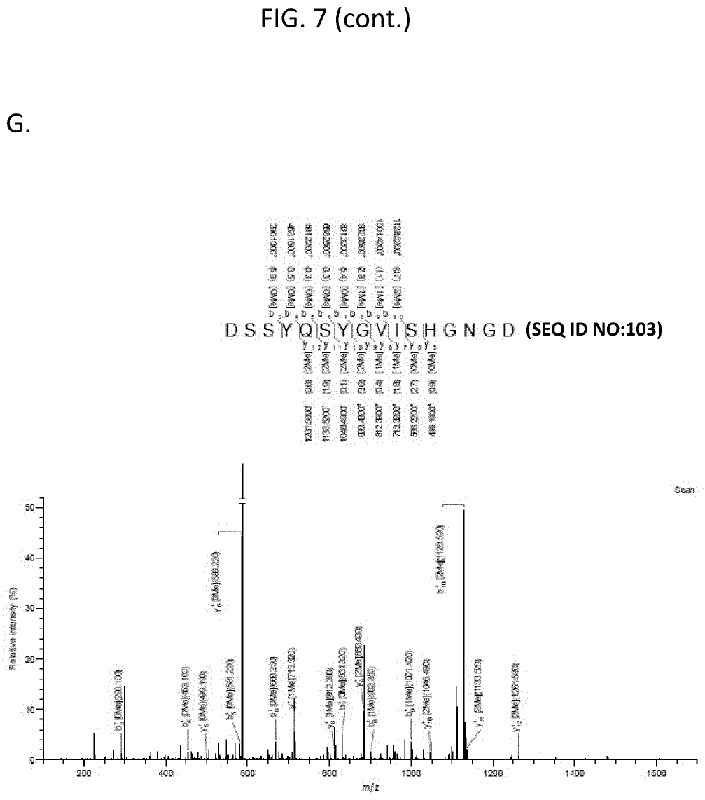

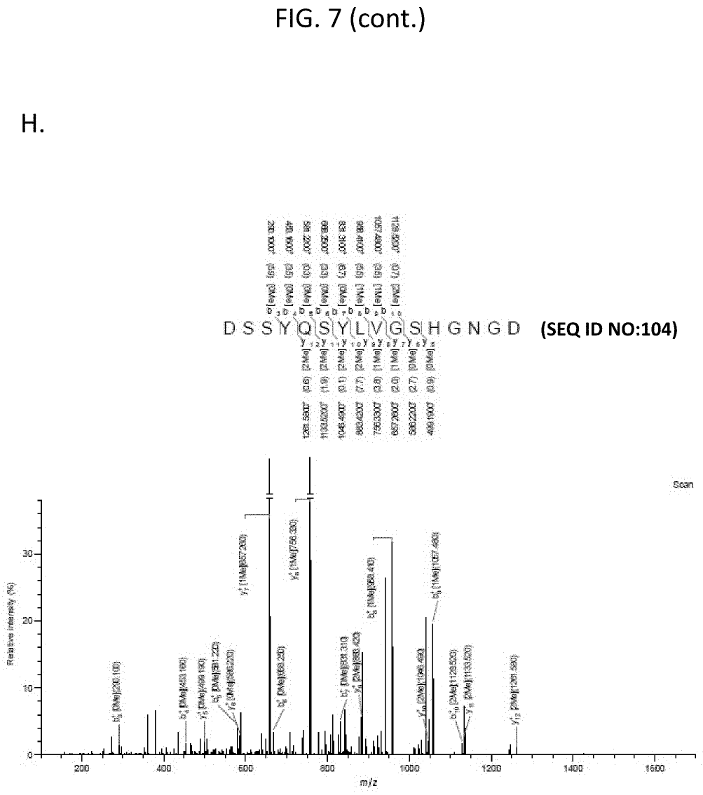

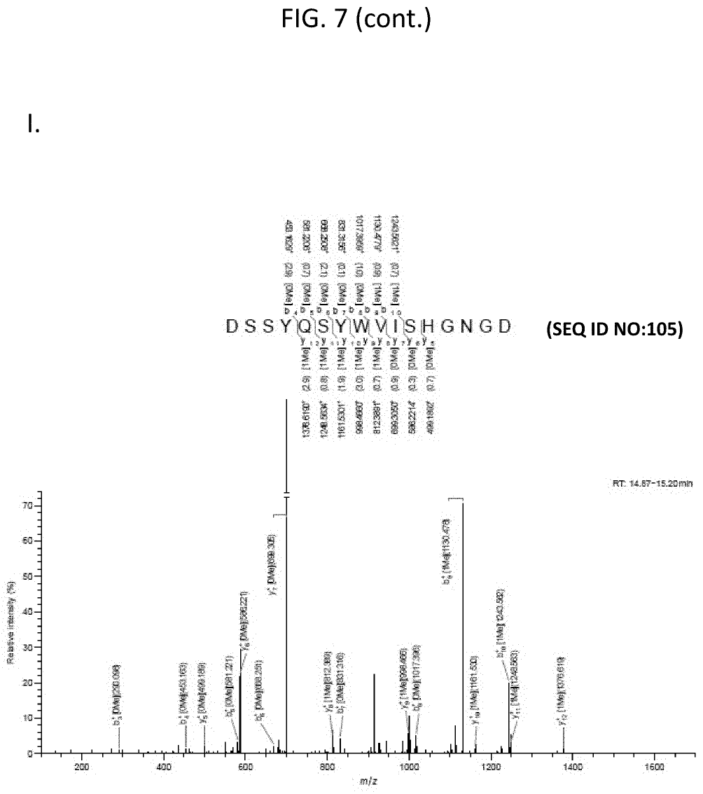

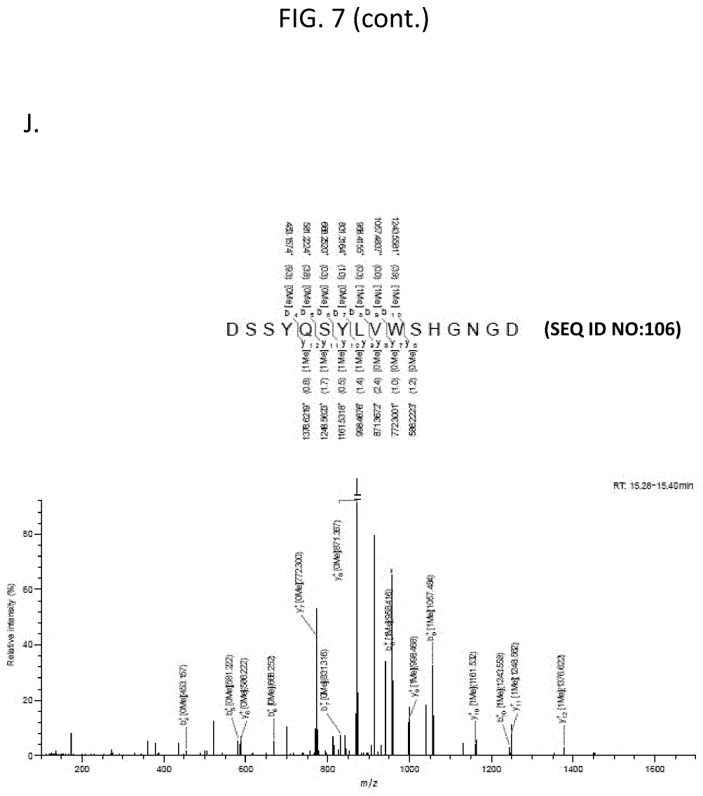

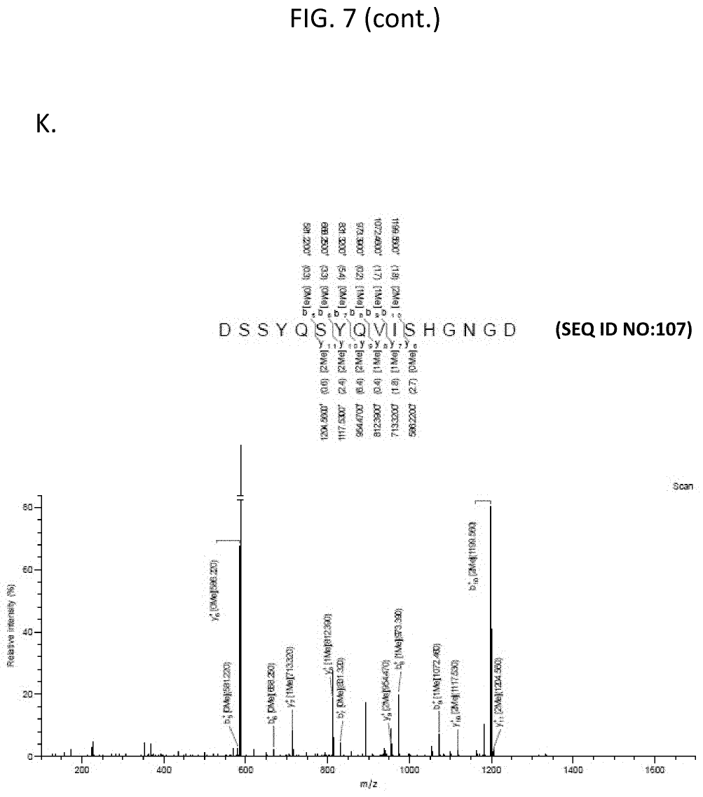

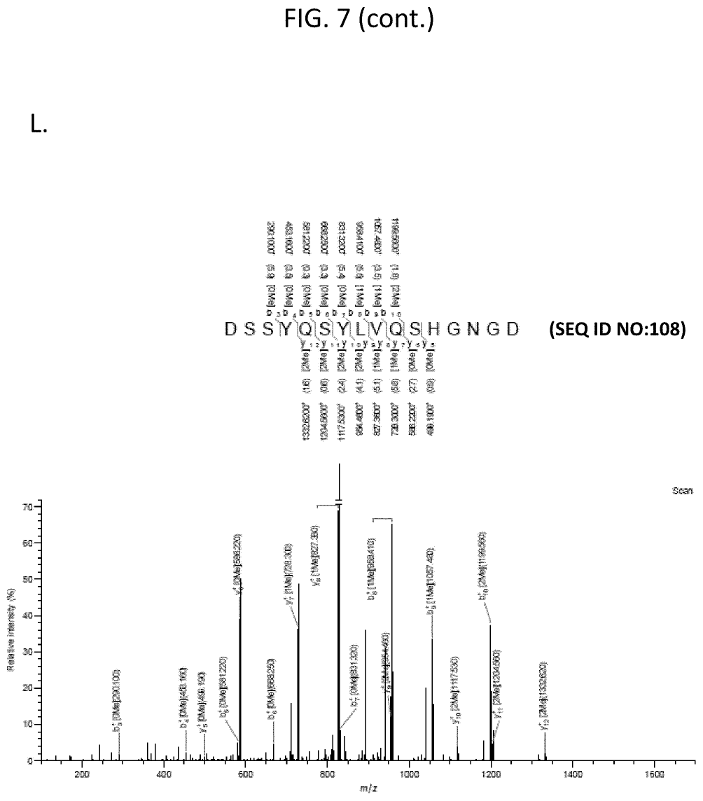

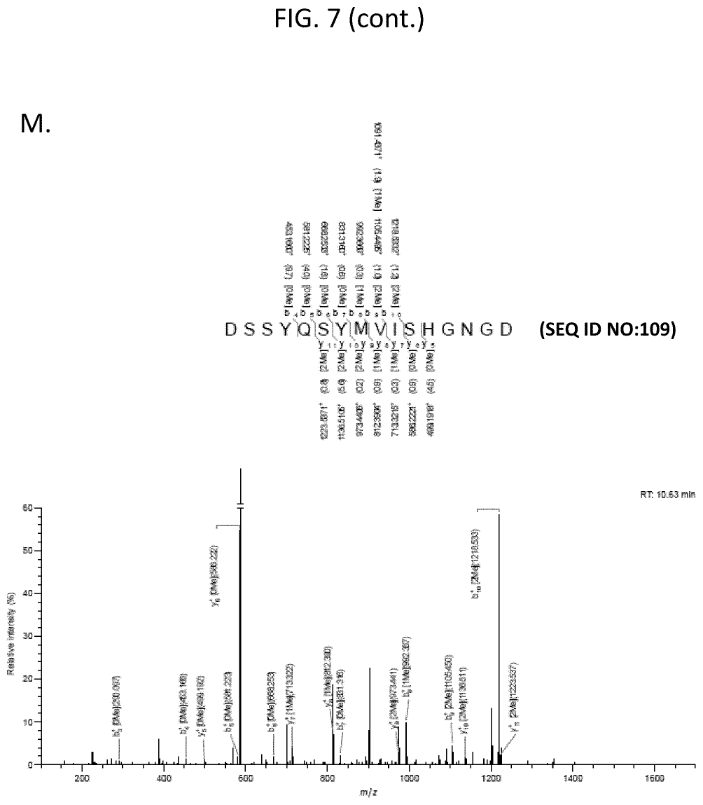

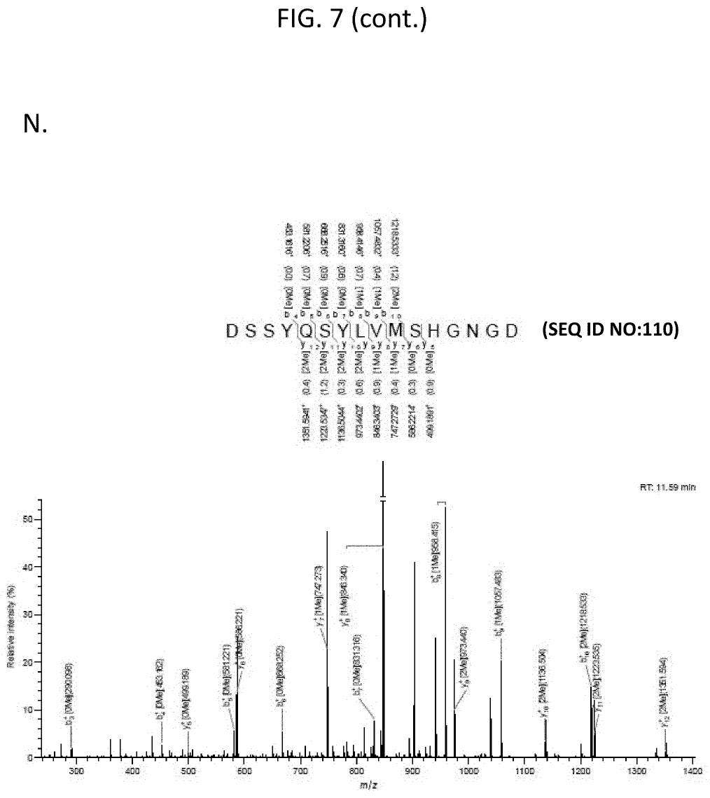

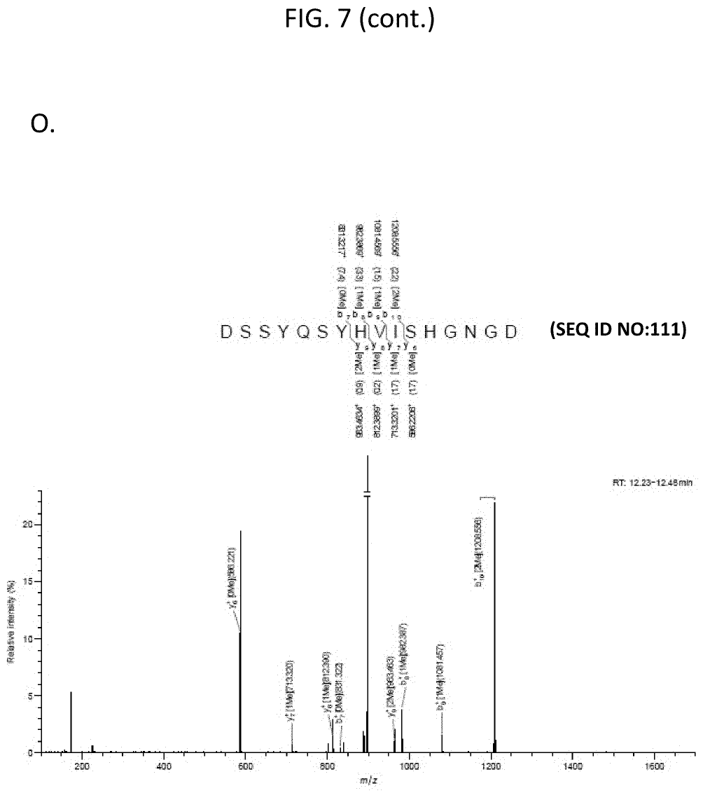

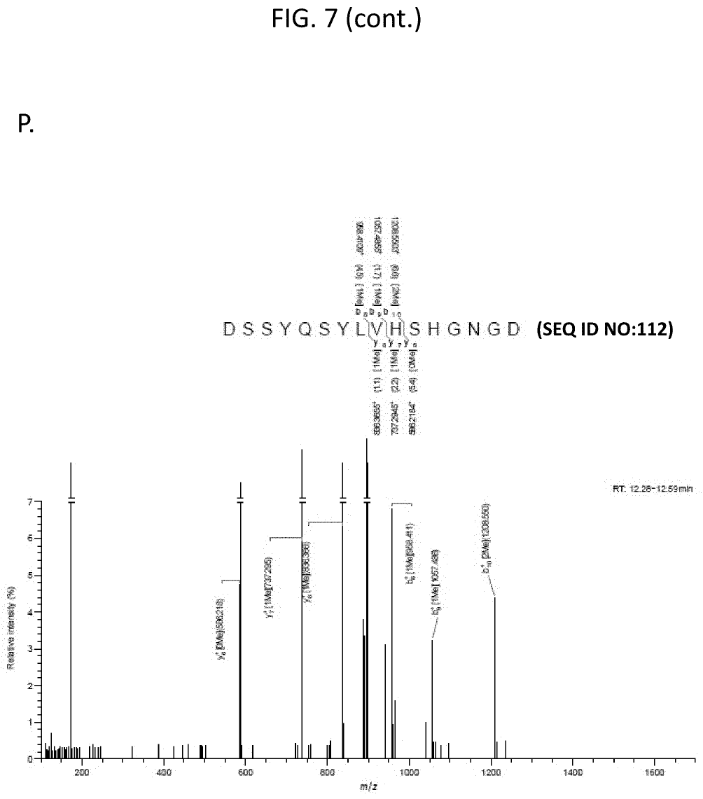

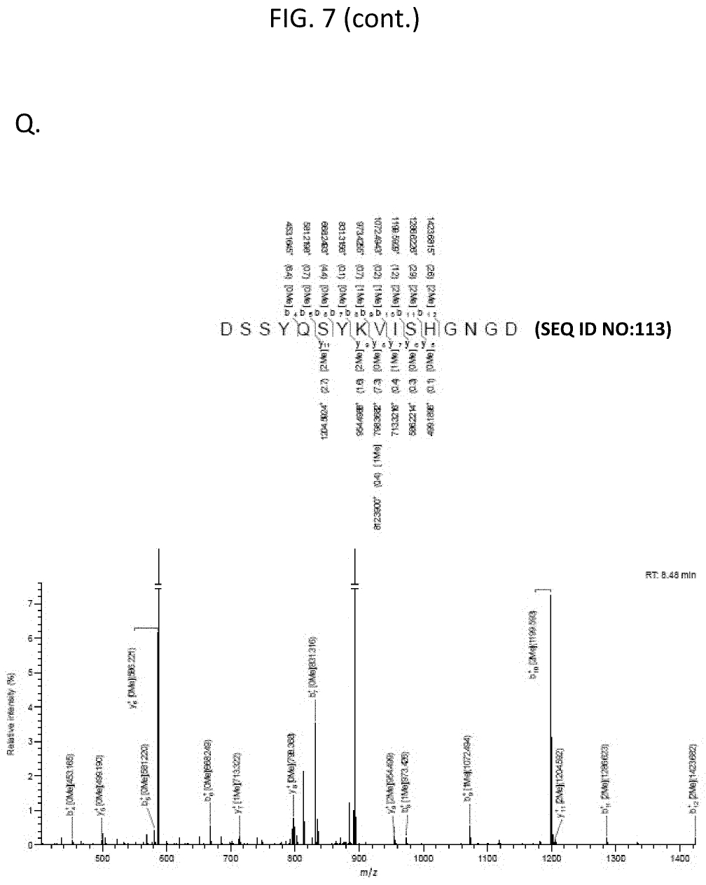

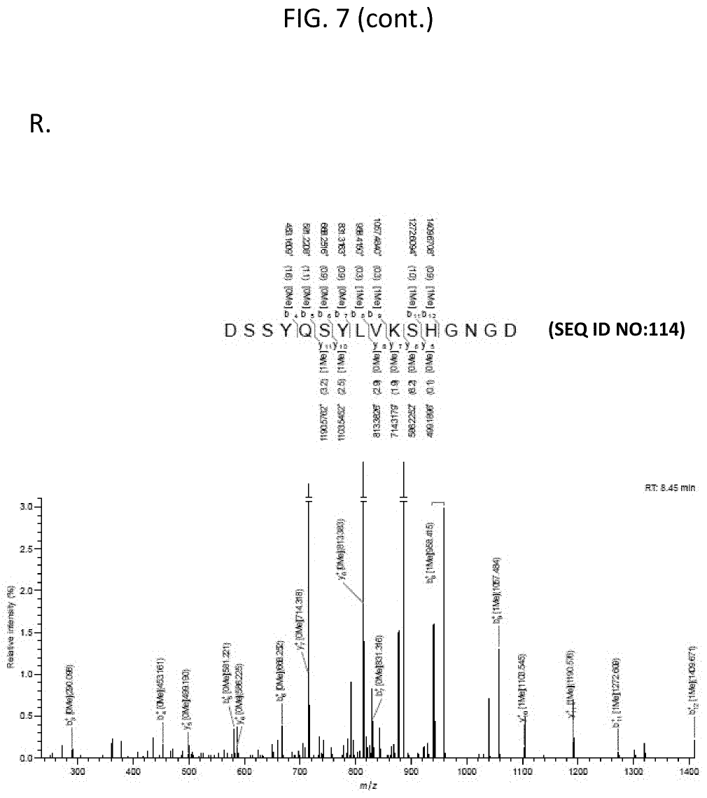

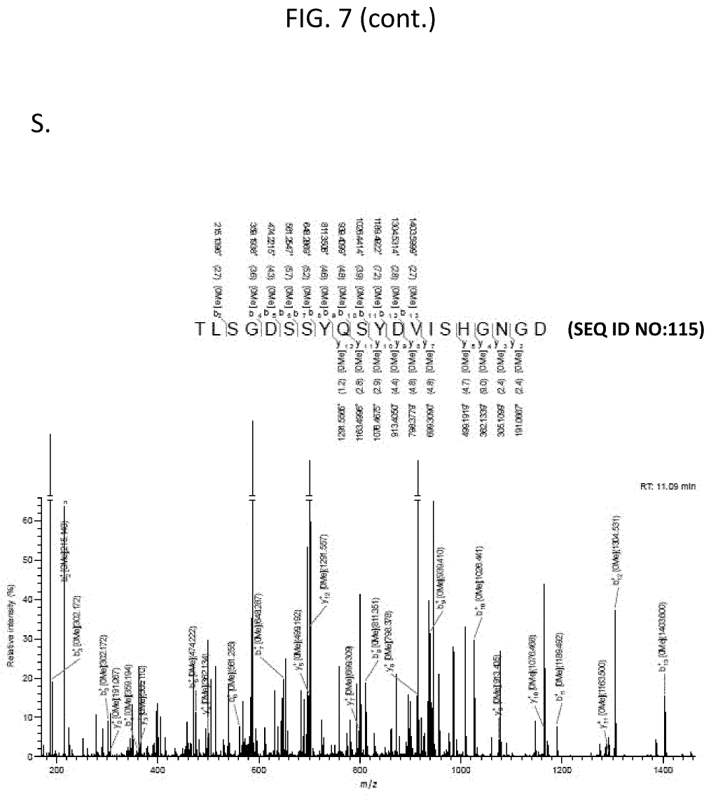

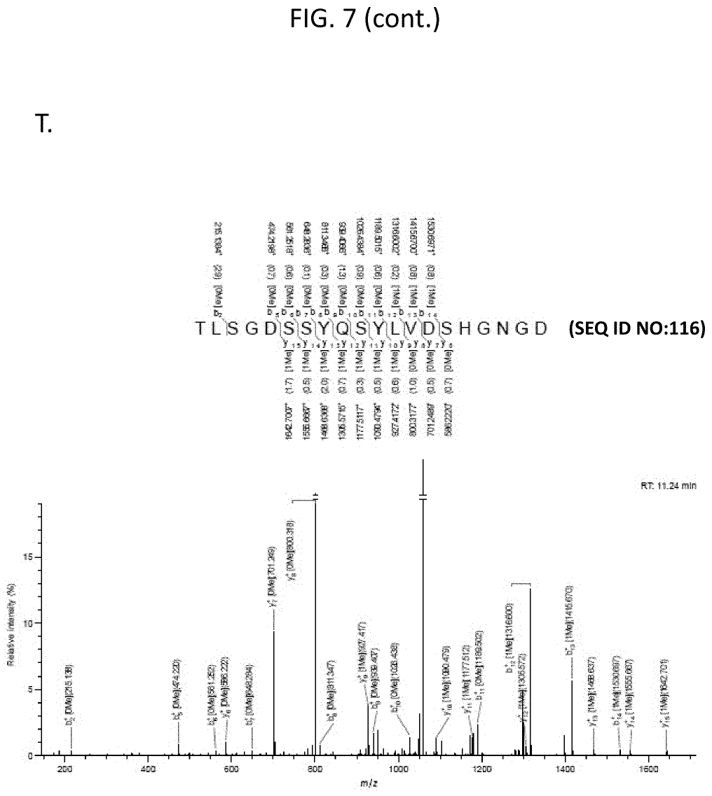

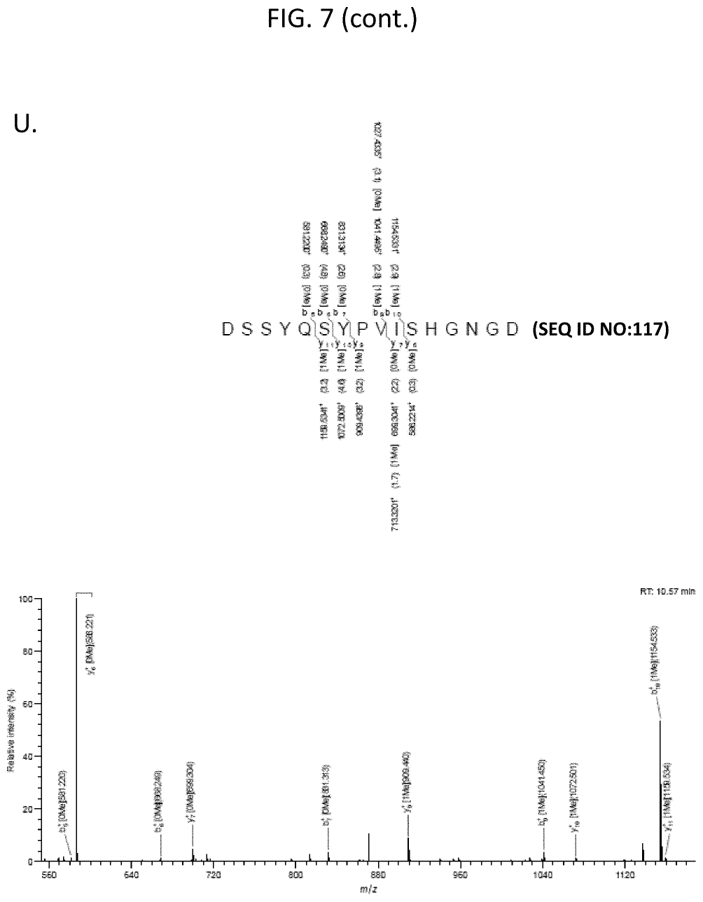

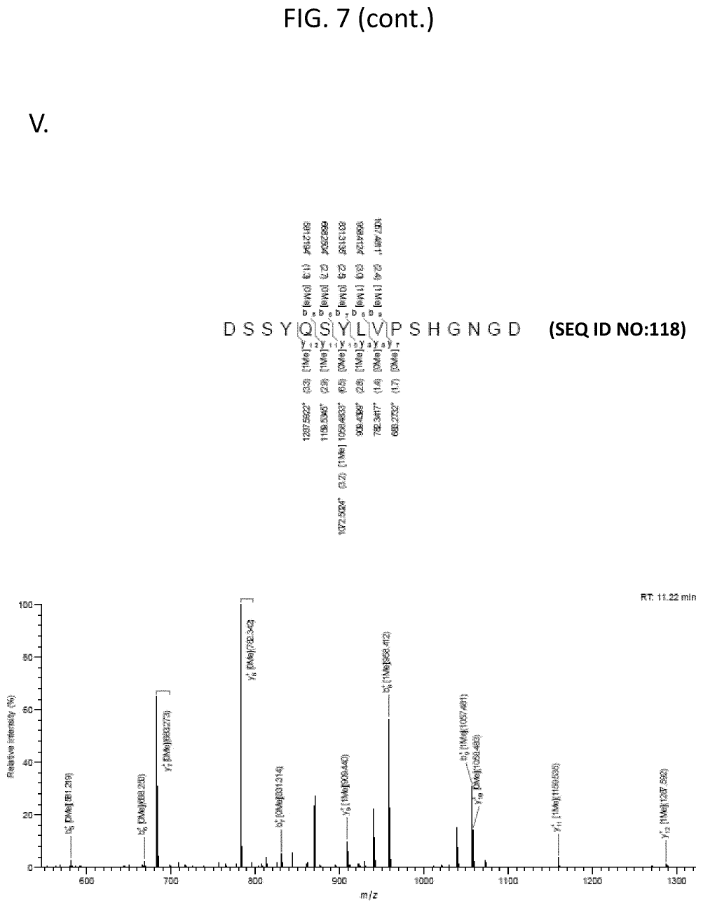

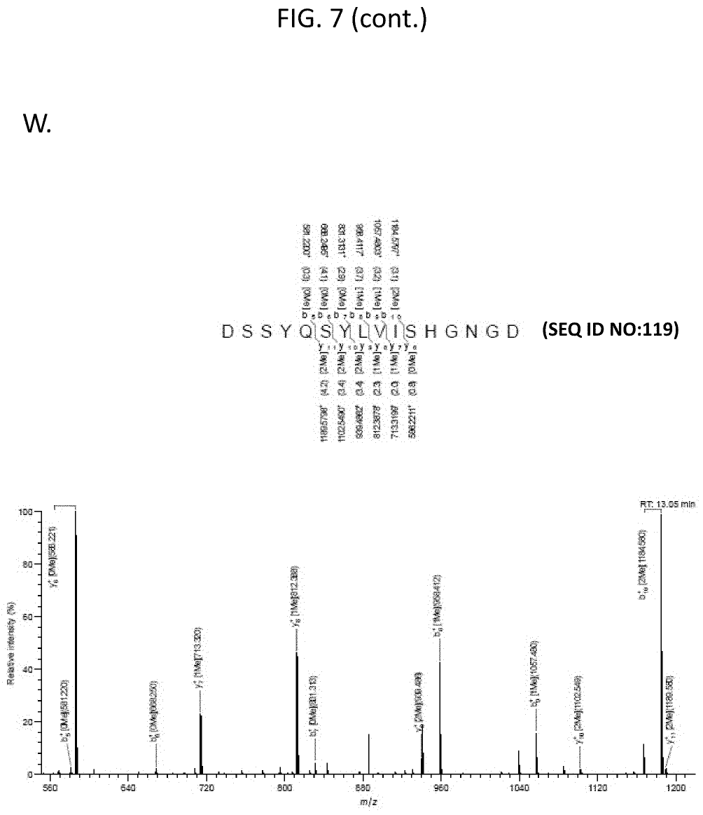

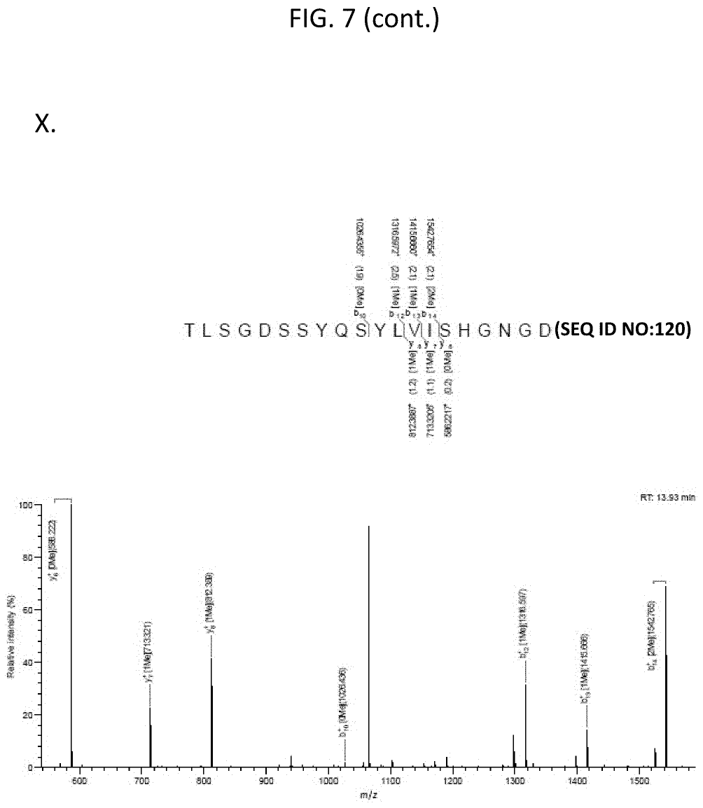

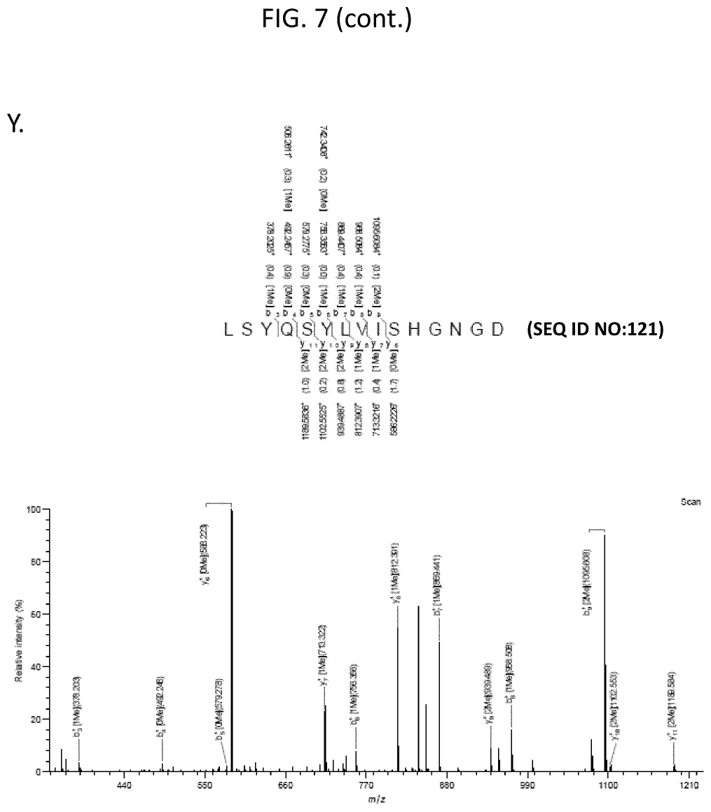

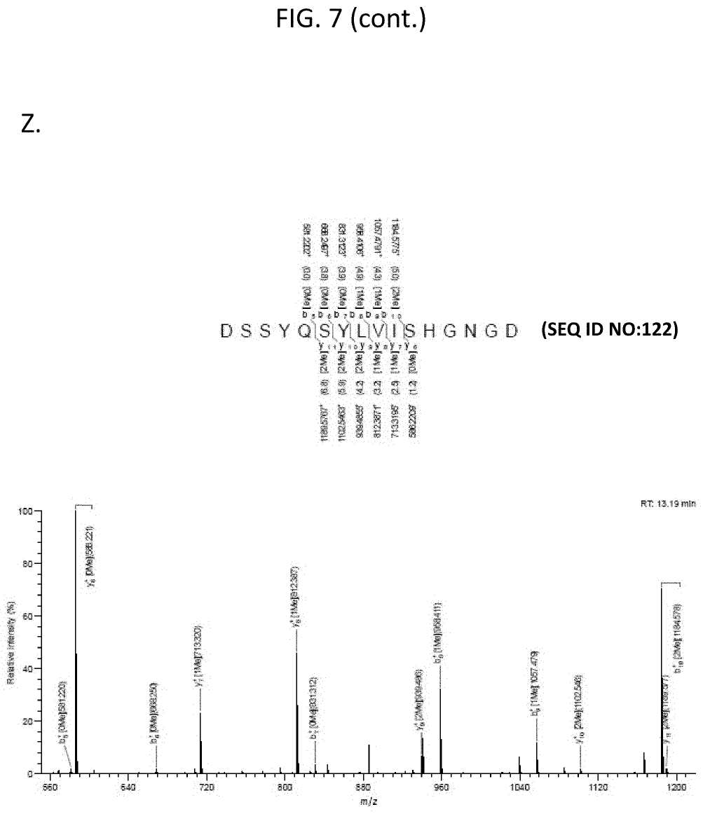

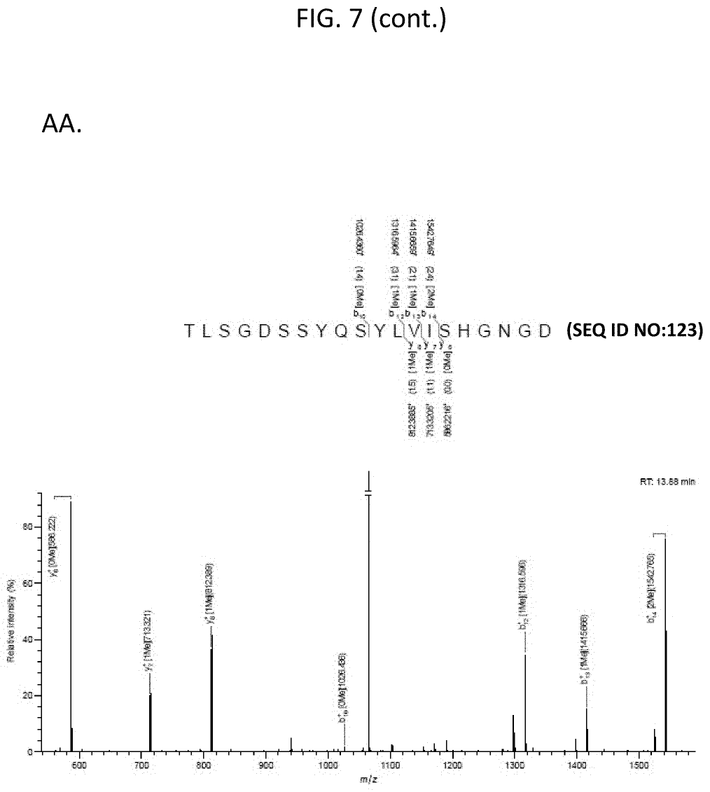

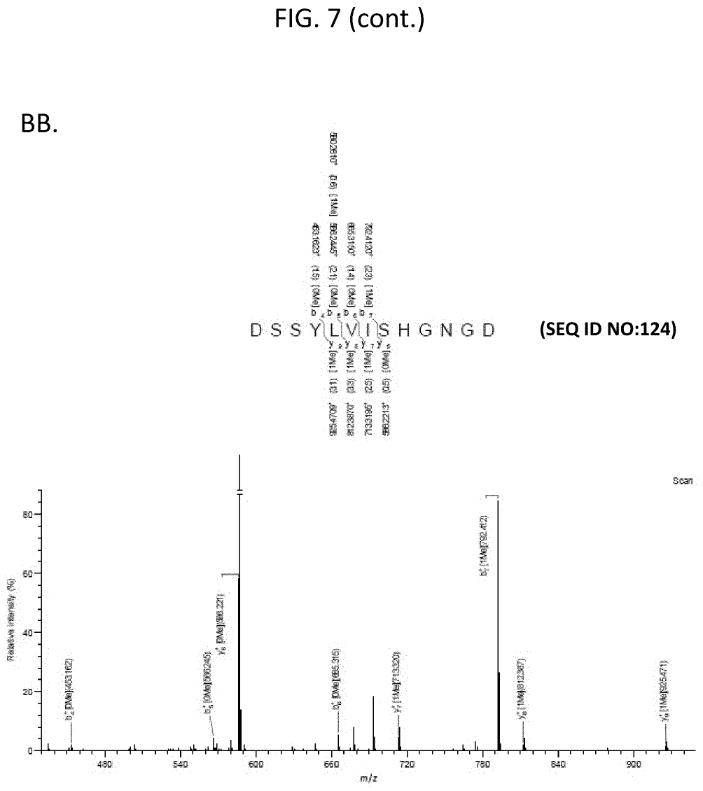

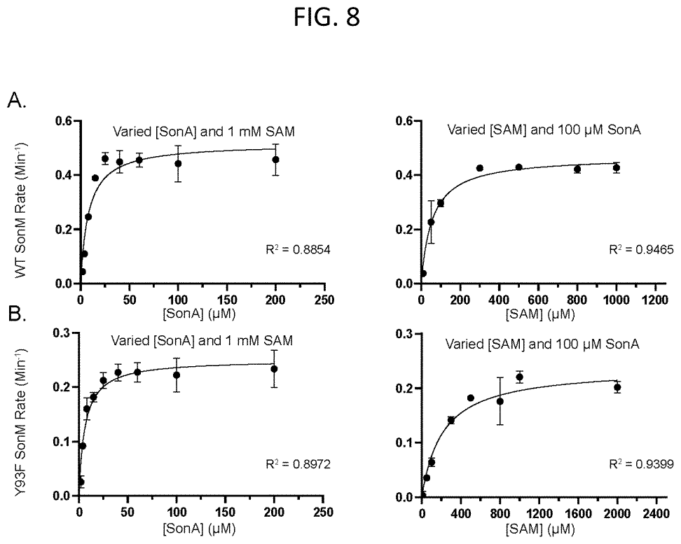

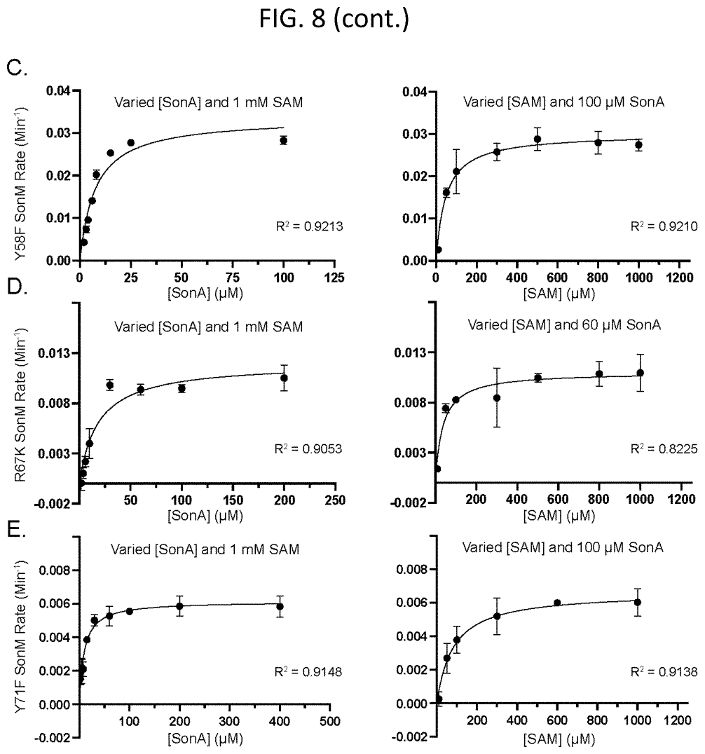

Methods for Data Described in FIGS. 7 and 8.

The gene coding for His6-SonA was amplified from the co-expression construct using primers prFM1175 (5′-TTTAAGAAGGAGATATACATGCATCATCATCATCAT-3′; SEQ ID NO:49)) and prFM1176 (5′-AGTGCGGCCGCAAGCTTGTTAATCACCATTACCATG-3′; SEQ ID NO:50). The gene coding for SonM was amplified and his6 tag was added from the co-expression construct using primers prFM1177 (5′-TAAGAAGGAGATATACATGCATCATCATCATCATCACAGCAGCATGGGATCACT CGTC-3′; SEQ ID NO:51) and prFM1178 (5′-AGTGCGGCCGCAAGCTTGTTATCCCAAATCTTCGGG-3′; SEQ ID NO:52). After verification by agarose gel electrophoresis, the PCR products were cleaned up using a kit (Thermo Scientific). The backbone (pET28b) was prepared by digesting with NcoI-HF and SalI-HF (NEB), treating with Antarctic Phosphatase (NEB), and extracting the digested backbone from an agarose gel (NEB Monarch kit). Gibson assembly for both constructs was performed using Hafiz DNA Assembly Master Mix (NEB) according to the manufacturer's instructions. Resultant colonies were screened by colony PCR using primers T7_fw (5′-TAATACGACTCACTATAGGG-3′; SEQ ID NO:47) and T7_rv (5′-GCTAGTTATTGCTCAGCGG-3′; SEQ ID NO:48). Positive hits were sequence verified by ACGT using Sanger sequencing and the same colony PCR primers.

Heterologous expressions were conducted in E. coli cells BL21(DE3). Saturated overnight culture (10 mL) in LB with 50 μg/mL kanamycin was used to inoculate 1 L of TB with 50 μg/mL kanamycin in a 2.5 L baffled Ultra Yield flask (Thomson Scientific). The 1 L culture was incubated in a 37° C. shaker until the OD₆₀₀ reached approximately 0.7, at which time the culture was cold shocked in an ice bath for 30-60 min. After cold shocking, the culture was induced with 200 mM IPTG and placed in a 16° C. shaker for 24 hrs. After 24 hrs, the cells were harvested by centrifugation at 4000×g for 30 min at 4° C., snap frozen in liquid nitrogen, and stored at −80° C. until use.

For protein purification by nickel affinity chromatography, frozen cells were thawed on ice and then resuspended to homogeneity in ice cold lysis buffer (300 mM NaCl, 50 mM HEPES, 20 mM imidazole, 10% glycerol, pH 8.0) with 4 mL of buffer for every 1 g of wet cell mass. After resuspension, lysozyme was then added to a final concentration of 1 mg/mL and incubated on ice for 30 min. After lysozyme treatment, cells were further lysed by sonication. After sonication, lysate was clarified by centrifugation at 15,000×g for 45 min at 4° C. The soluble protein from the clarified supernatant was then batch-bound to nickel-NTA resin (GoldBio) for 60 min on a rotator at 4° C. After binding, resin was added to a 5 mL fritted column, washed with 10 column volumes of lysis buffer, and the protein was eluted in lysis buffer with 250 mM imidazole. For subsequent gel filtration chromatography, protein was concentrated, sterile filtered and loaded onto a HiLoad 16/600 Superdex 200 pg size exclusion column was used at a flow rate of 1 ml/min of lysis buffer without imidazole. Protein was analyzed by SDS-PAGE gel, fractions were pooled and concentrated using Amicon Ultra centrifugal filter columns (MilliporeSigma). Concentrations were measured by Bradford assay and proteins were snap frozen in liquid nitrogen and stored at −80° C. until use. When using frozen protein, all samples were thawed on ice, centrifuged at top speed in a microcentrifuge at 4° C. for 10 min, aggregate removed by transferring supernatant to a fresh tube, and the concentration re-measured.

For the coupled-enzyme kinetics assay, plasmids for expressing S-adenosylhomocysteine nucleosidase (SAHN; Uniprot POAF12) and adenine deaminase (ADE; Uniprot P31441) with N-terminal his6 tags were acquired from the ASKA collection.²³ SAHN was expressed and purified as above with the addition of 1 mM DTT in all buffers. During the expression of ADE, to replace the Fe²⁺ metal with Mn²⁺ in the active site, 20 μM 2,2′-dipyridyl and 1.0 mM MnCl₂ were added at the time of induction.²⁴ Other expression and purification steps for ADE were carried out in the same manner as for SAHN. Glutamate dehydrogenase (GDH) and ammonia assay reagent were used from the Ammonia Detection Kit (Millipore Sigma AA0100) according to previously established methods.²⁵ For use in the kinetics assays, SAM was purified by HPLC using a BUCHI PrepChrom C-700 instrument and BUCHI FlashPure EcoFlex C18 Column (140000048). A flow rate of 10 mlimin was used with a gradient of: Solvent (A) H₂O with 0.1% formic acid and Solvent (B) acetonitrile. The gradient used was Solvent A) 95% 0.5 min, 95%-5% 15 min, 5% 2 min. SAM was purified to ˜97-98.5% purity when measured by our assay. Kinetic experiments were conducted in a clear, flat-bottomed 96-well plate in a SpectraMax IDS (Molecular Devices, Inc). Methyl transfer was measured by monitoring the decrease in absorbance at 340 nm (corresponding to the loss of NADPH in the coupled-enzyme assay). Three replicates for each condition were used, and reads were taken every 30 or 40 secs. Upon assembling all assay components except the methyltransferase in the plate wells, absorbance values were collected for 10-15 minutes prior to the addition of the methyltransferase to start the reaction. The absorbance data was used to calculate the concentration of NADPH at each time point with Beers' Law and the reported extinction coefficient of NADPH, 6220 M⁻¹. The concentration of the final reading before addition of the methyltransferase was used to subtract all successive concentration values from, making the curve reflect product formation over time. The slope was taken over the linear range of this curve giving the velocity of product formation (μM/min). The velocity of the three negative control replicates (lacking the varied substrate) were averaged and subtracted from the velocity of each individual replicate to account for background SAM degradation. These velocity values were then divided by the enzyme concentration used giving the rate of product formation (min⁻¹) and plotted with their respective substrate concentrations in GraphPad Prism to produce the substrate-velocity curve. A non-linear regression analysis was used to fit the data to the Michaelis-Menten equation and give values for the desired kinetic constants, Vmax, Kcat, and Km or Ki, where appropriate.

The SonM and SonA mutants that were tested in FIG. 7 were generated by PCR site directed mutagenesis.

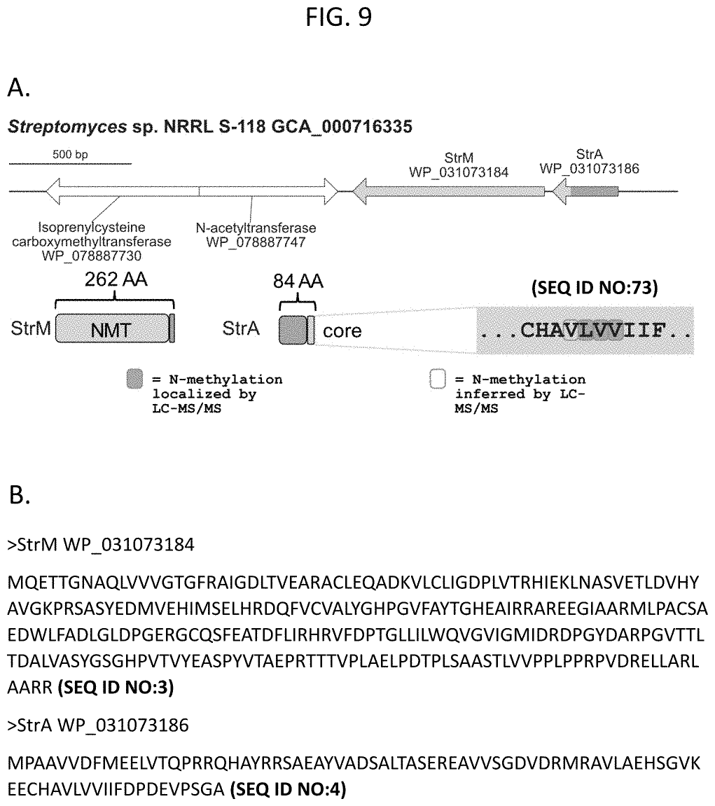

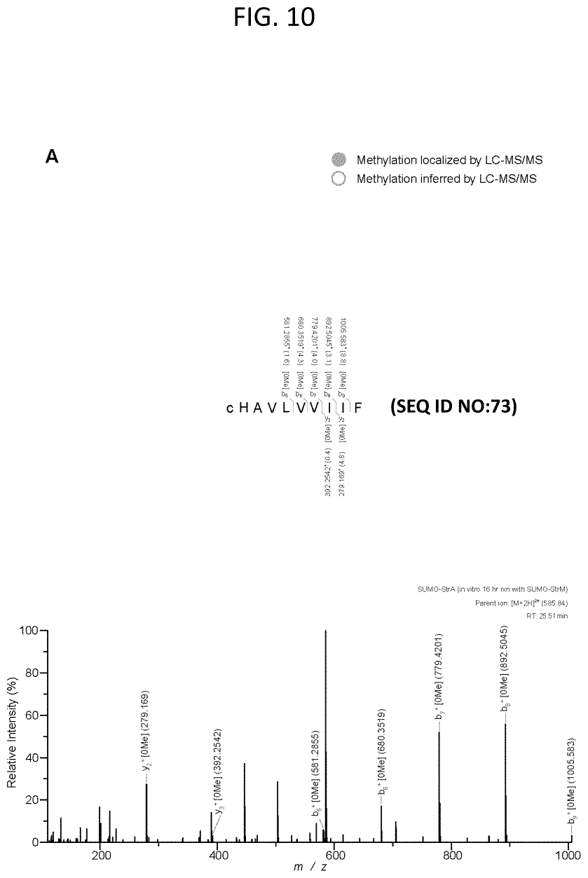

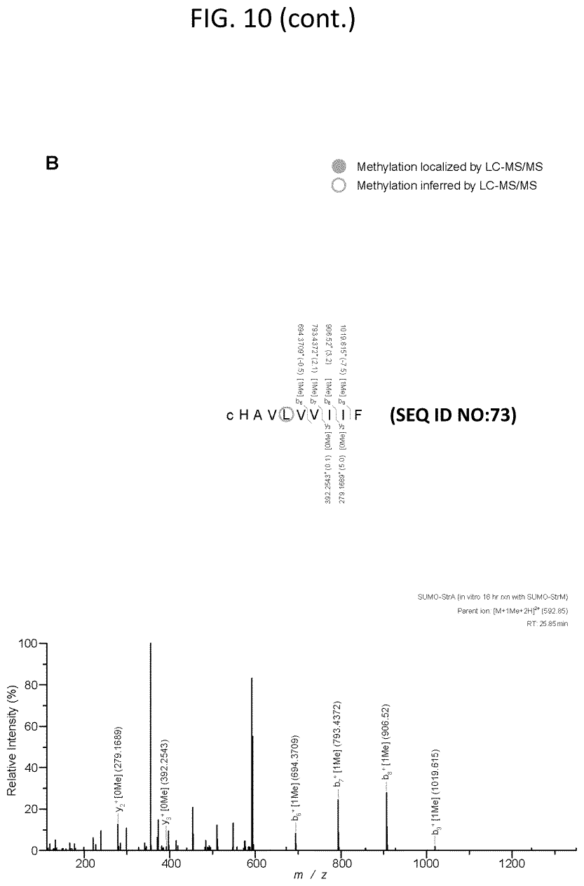

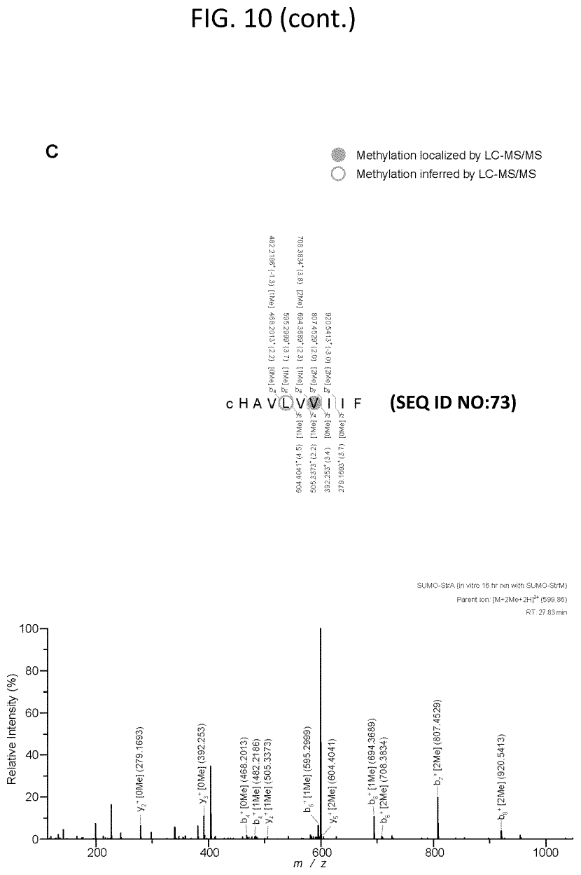

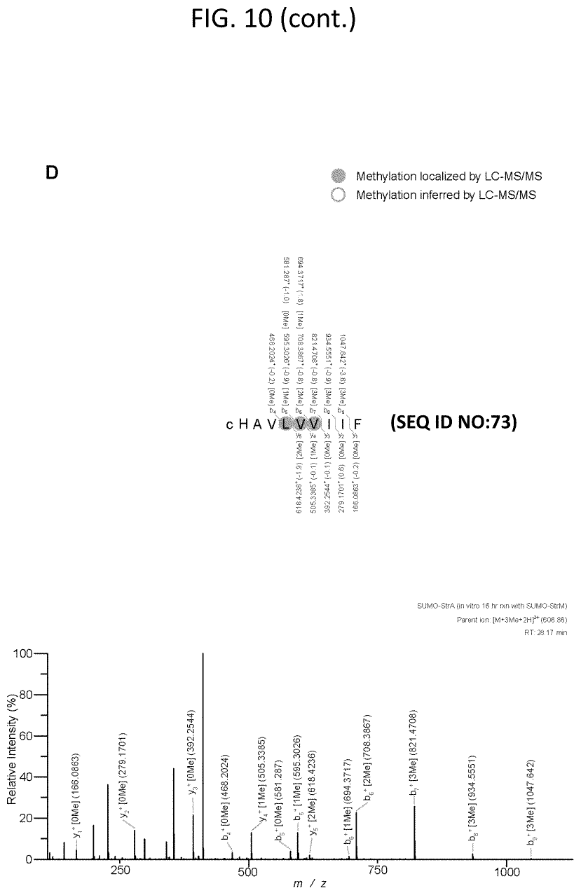

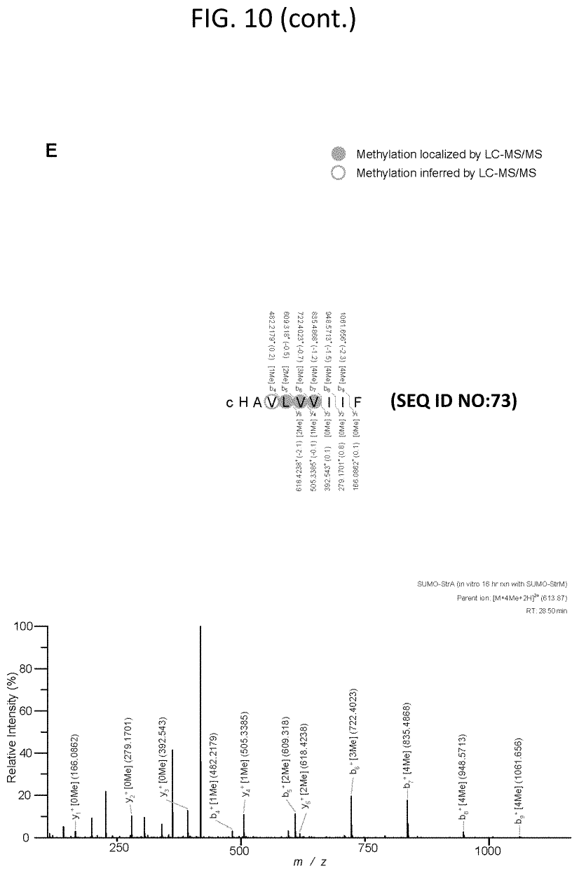

Methods for Data Described in FIGS. 9 and 10.

N-Terminal histidine and SUMO tags were cloned in front of strA and strM. Construct pET28b-his6-SUMO backbone was digested with NdeI and BamHI, treated with Antarctic phosphatase, and the band was extracted from an agarose gel using a kit (Thermo Scientific). Gene fragments for strA and strM were codon optimized for expression in E. coli and purchased as gBlocks. The strA gBlock was amplified with primers prmMRJ068 (5′-ATATAACATATGCCGGCGGC-3′; SEQ ID NO:53) and prmMRJ069 (5′-TTATATGGATCCTTACGCACCGCTCGG-3′; SEQ ID NO:54) to add NdeI and BamHI cut sites on the termini. The PCR product was verified by agarose gel electrophoresis, digested with NdeI and BamHI, and the reaction was cleaned up using a kit (Thermo Scientific). The strM gBlock was amplified with primers prmMRJ066 (5′-ATATAACATATGCAGGAGACCACCG-3′; SEQ ID NO:55) and prmMRJ067 (5′-TTATATGGATCCTTAACGACGCGCCG-3′; SEQ ID NO:56) to add NdeI and BamHI cut sites on the termini. The PCR product was verified by agarose gel electrophoresis, digested with NdeI and BamHI, and the reaction was cleaned up using a kit (Thermo Scientific). T4 DNA ligase was used to ligate the sticky overhangs into the prepared plasmid backbone. Resultant colonies were screened by colony PCR using primers T7_fw (5′-TAATACGACTCACTATAGGG-3′; SEQ ID NO:47) and T7_rv (5′-GCTAGTTATTGCTCAGCGG-3′; SEQ ID NO:48). Positive hits were sequence verified by ACGT using Sanger sequencing and the same colony PCR primers.

Heterologous expressions were conducted in E. coli cells BL21(DE3). Saturated overnight culture (10 mL) in LB with 50 μg/mL kanamycin was used to inoculate 1 L of TB with 50 μg/mL kanamycin in a 2.5 L baffled Ultra Yield flask (Thomson Scientific). The 1 L culture was incubated in a 37° C. shaker until the OD₆₀₀ reached approximately 0.7, at which time the culture was cold shocked in an ice bath for 30-60 min. After cold shocking, the culture was induced with 200 mM IPTG and placed in a 16° C. shaker for 24 hrs. After 24 hrs, the cells were harvested by centrifugation at 4000×g for 30 min at 4° C., snap frozen in liquid nitrogen, and stored at −80° C. until use.

For protein purification by nickel affinity chromatography, frozen cells were thawed on ice and then resuspended to homogeneity in ice cold lysis buffer (300 mM NaCl, 50 mM sodium phosphate, 20 mM imidazole, 10% glycerol, pH 8.0) with 4 mL of buffer for every 1 g of wet cell mass. After resuspension, lysozyme was then added to a final concentration of 1 mg/mL and incubated on ice for 30 min. After lysozyme treatment, cells were further lysed by sonication. After sonication, lysate was clarified by centrifugation at 15,000×g for 45 min at 4° C. The soluble protein from the clarified supernatant was then batch-bound to nickel-NTA resin (GoldBio) for 60 min on a rotator at 4° C. After binding, resin was added to a 5 mL fritted column, washed with 10 column volumes of lysis buffer, and the protein was eluted in lysis buffer with 250 mM imidazole. For subsequent gel filtration chromatography, protein was concentrated, sterile filtered and loaded onto a HiLoad 16/600 Superdex 200 pg size exclusion column was used at a flow rate of 1 ml/min of lysis buffer without imidazole. Protein was analyzed by SDS-PAGE gel, fractions were pooled and concentrated using Amicon Ultra centrifugal filter columns (MilliporeSigma). Concentrations were measured by Bradford assay and proteins were snap frozen in liquid nitrogen and stored at −80° C. until use. When using frozen protein, all samples were thawed on ice, centrifuged at top speed in a microcentrifuge at 4° C. for 10 min, aggregate removed by transferring supernatant to a fresh tube, and the concentration re-measured.

Split borosin methyltransferase and precursor proteins were expressed and purified as described above in separate plasmids. Proteins were dialyzed into a buffer containing 50 mM HEPES, 300 mM NaCl, 10% glycerol, pH 8.0. Reactions were conducted in 100 μL final volumes with saturating amounts of SAM (dissolved in 0.5 mM HEPES pH 8.0). Precursor (25 μM) was used in all samples. Reactions were incubated at room temperature for 16 hrs and quenched with SDS sample buffer and boiled prior to in-gel digestion and HPLC-MS/MS analysis.

Briefly, the band corresponding to his6-SUMO-StrA was extracted from an SDS-PAGE gel, cut into ˜2 mm×2 mm pieces and placed in 1.5 mL LoBind tubes (Eppendorf). Gel cubes were then washed with a 1:1 ratio of 100 mM ammonium bicarbonate (ABC): acetonitrile (ACN) three times until gel pieces appeared clear. After dye removal, they were then dehydrated in 100% ACN until semi-opaque (˜30 sec), and the ACN was subsequently discarded. The gel pieces were rehydrated with a solution containing 50 mM ABC and 55 mM DTT and incubated in a 56° C. water bath for 60 min. DTT solution was subsequently removed and replaced with a solution containing 50 mM ABC and 55 mM iodoacetamide, at which point the tubes were placed in the dark at room temperature for 30 min. The iodoacetamide solution was removed. After rehydration in digest buffer (50 mM ABC and 1:50 units AspN protease (Promega) and GluC protease (Thermo Scientific)), gel pieces were placed on ice for 15 min and then were transferred to a 37° C. incubator overnight. The next day, excess liquid from the digest was collected and transferred to a new LoBind tube. Digested peptides were extracted from the gel pieces by first covering them with 60 μL of 50% ACN and 0.3% formic acid (FA) and incubating at room temperature for 15 min. After this incubation, the supernatant was recovered. This extraction was repeated with 60 μL of 80% ACN and 0.3% FA and the supernatant was recovered and placed into the same LoBind tube. The pooled peptide extractions were frozen at −80° C. for 30 min to deactivate the protease. After freezing, the extracted peptides were thawed and dried using a SpeedVac (Eppendorf). Dried peptides were reconstituted in 0.1% FA and purified/desalted using C18 ZipTips according to the manufacturer's instructions. Purified and desalted peptides were again dried using the SpeedVac and then reconstituted in 15-30 μl of 20% ACN, 0.1% FA, and transferred to glass vials for MS analysis. Peptide mass spectrometric analysis (LC-MS/MS HCD) LC-MS/MS measurements of digested peptides was performed as previously described.¹⁶ Briefly, data were obtained on a Thermo Scientific Fusion mass spectrometer furnished with a Dionex Ultimate 3000 UHPLC system with a nLC column (200 mm×75 μm) packed with Vydac 5-μm particles of 300 Å pore size (Hichrom Limited). Elutions used a linear gradient consisting of 0.1% FA in water (solvent A) and 0.1% FA in ACN (solvent B) at a flow rate of 0.3 μl/min. The column was initially equilibrated with 20% solvent B for 5 min and then subjected to a linear increase of solvent B to 85% over 32 min followed by a final elution step of 85% solvent B for 2 min. Mass spectra were acquired in positive-ion mode. Full MS was done at a resolution of 60,000 [automatic gain control (AGC) target, 4×10⁵; maximum ion trap (IT), 50 ms; range, 300 to 1800 m/z], and data-dependent and targeted MS/MS were both performed at a resolution of 15,000 (AGC target, 5×10⁵; maximum IT, 500 ms; isolation window, 2.2) using higher-energy collisional dissociation (HCD). HCD collision energies from 14-20% with steps of ±4% were used during LC-MS/MS measurements.

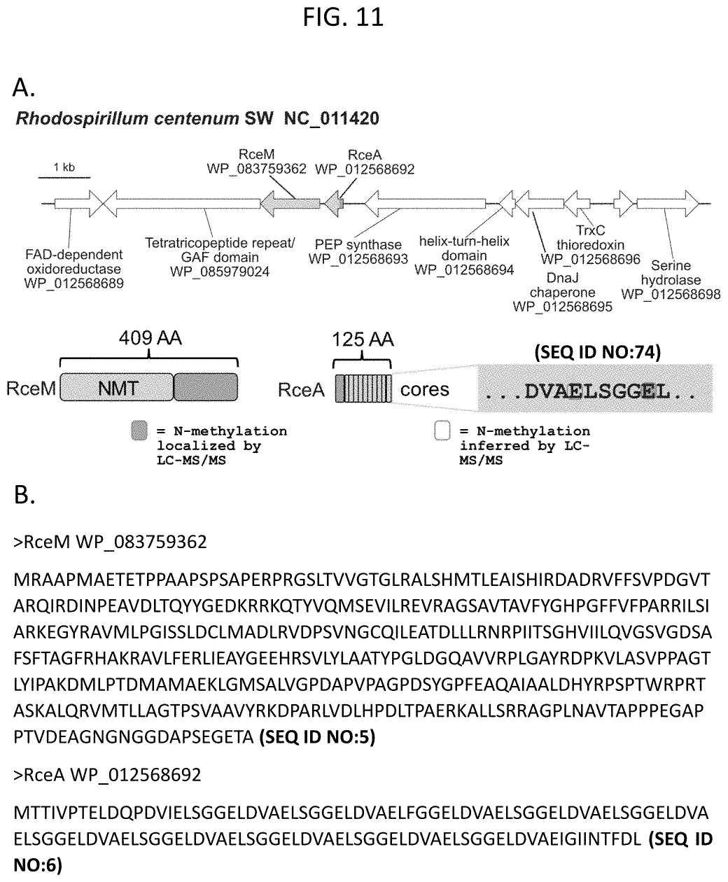

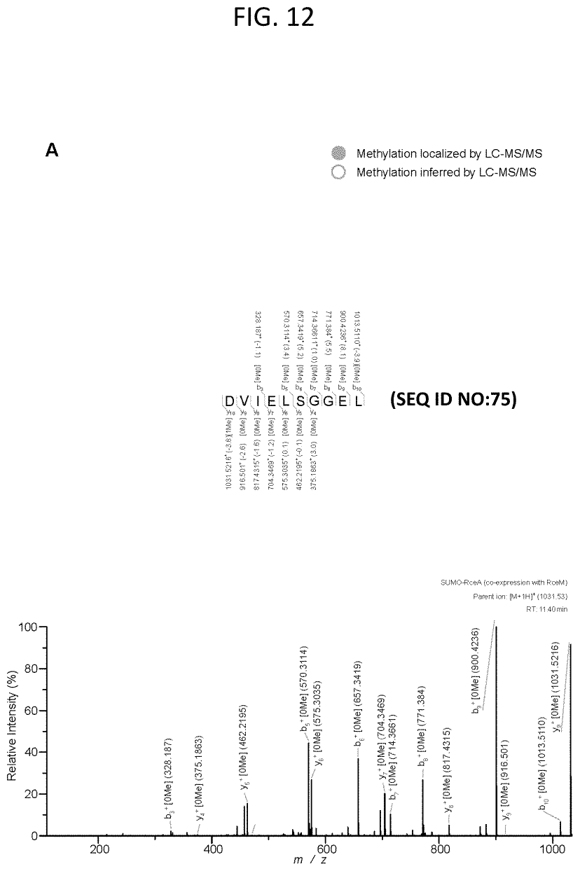

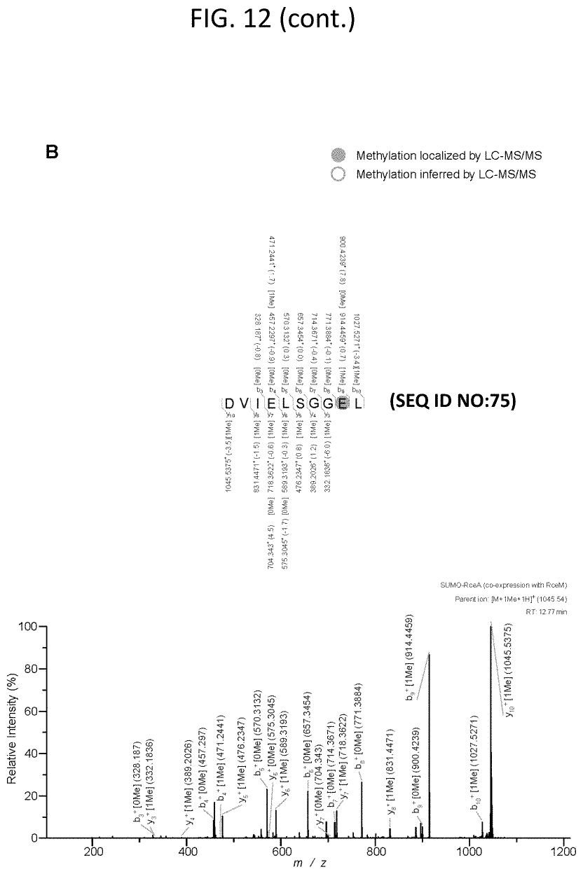

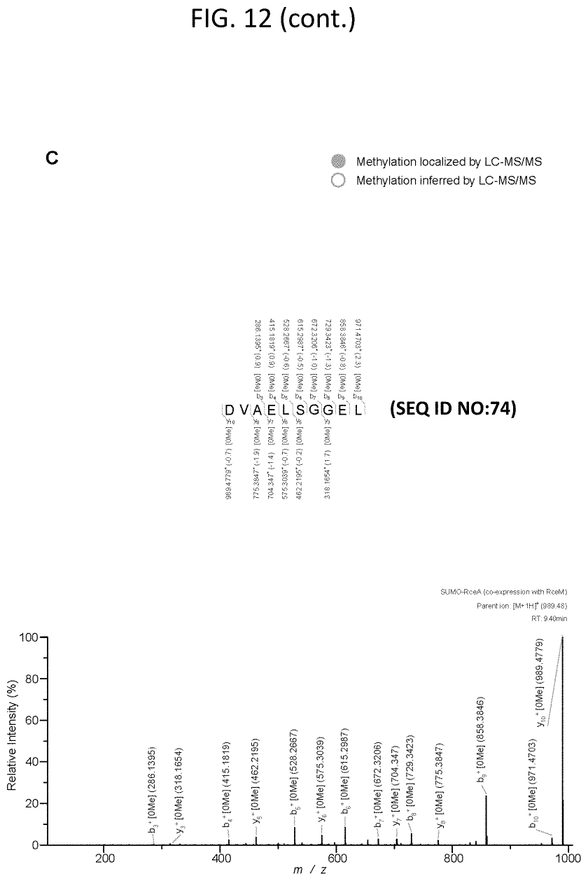

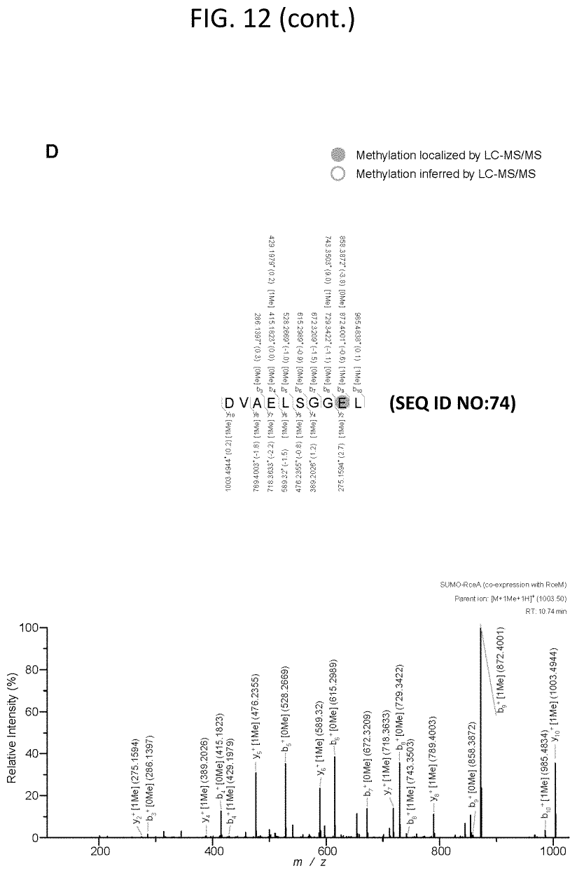

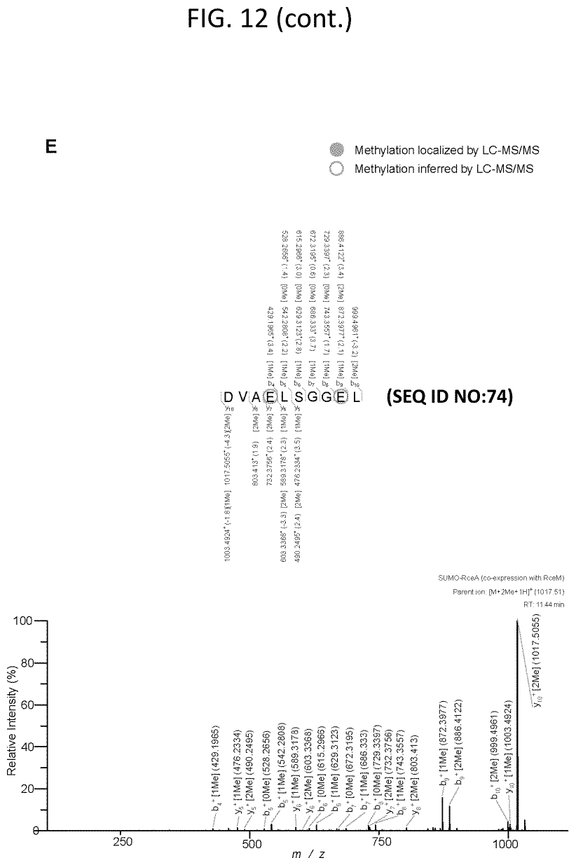

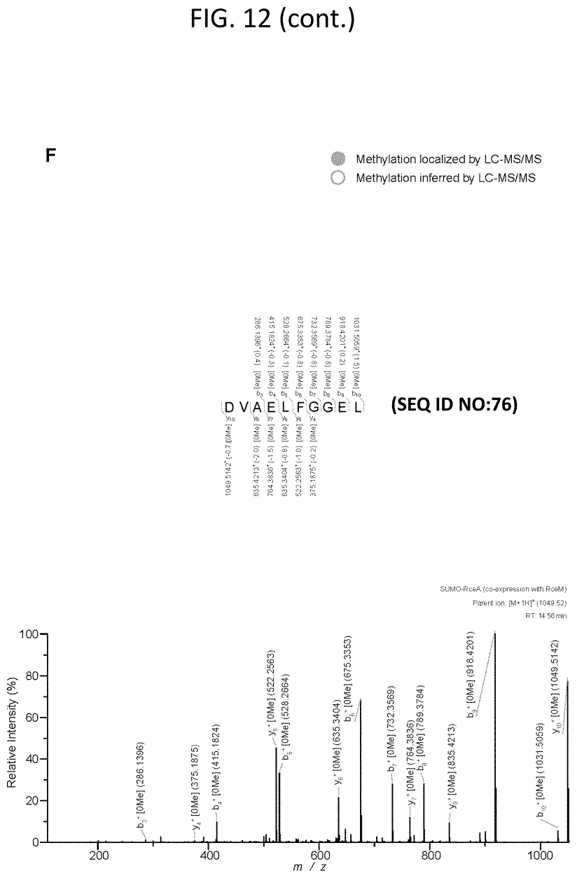

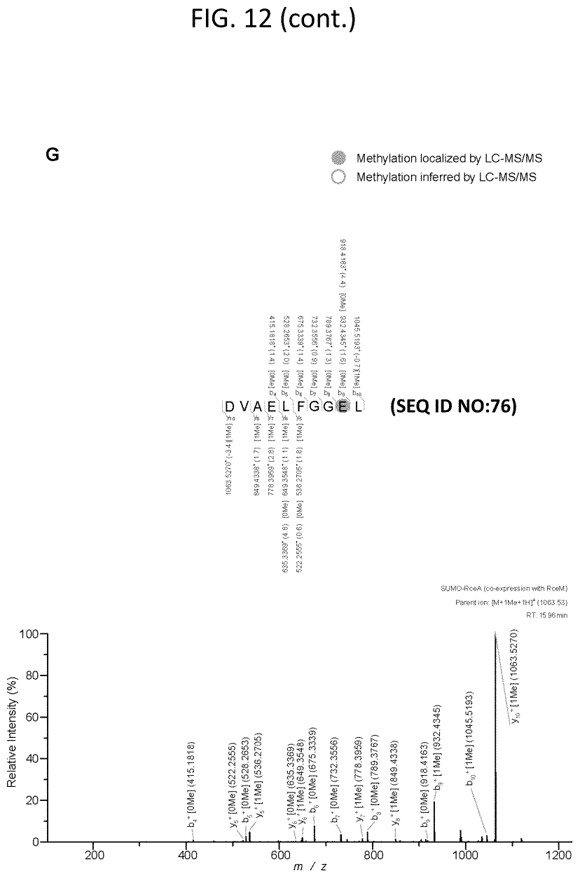

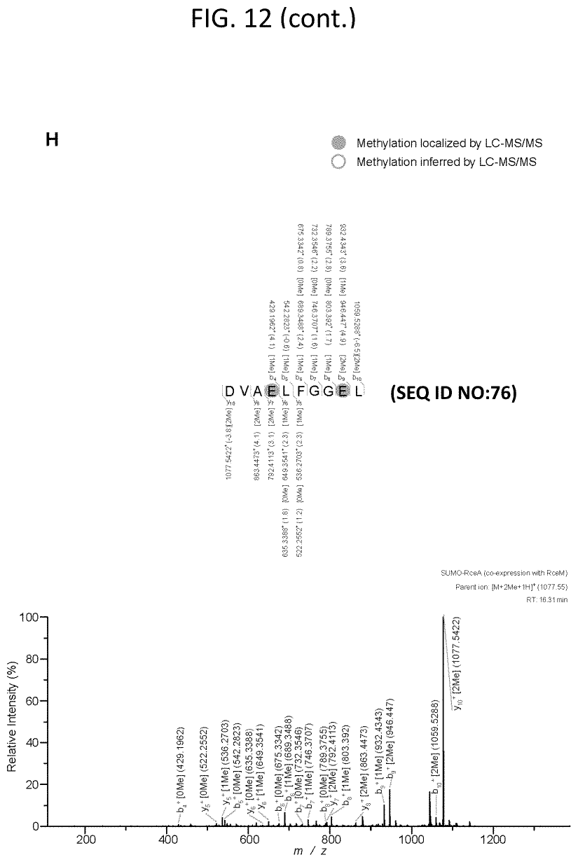

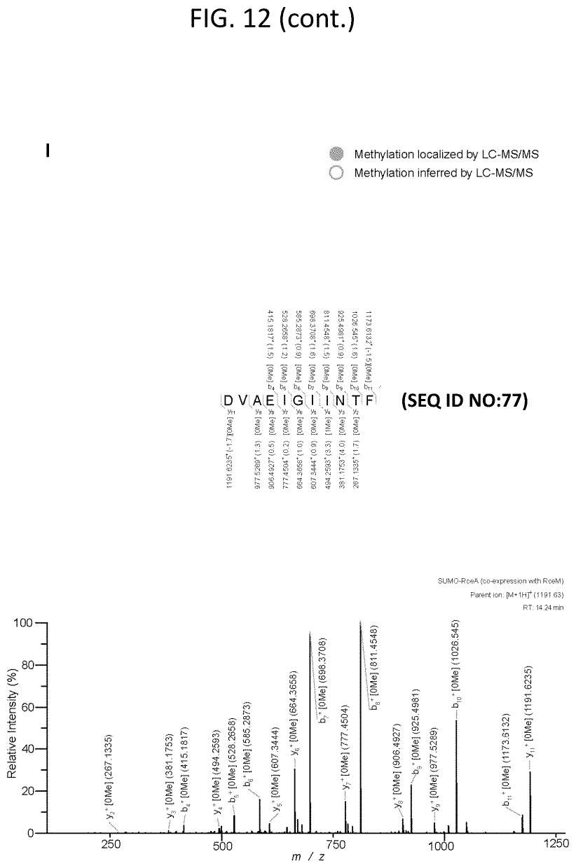

Methods for Data Described in FIGS. 11 and 12.

An N-terminal histidine and SUMO tag was cloned in front of rceA. Construct pET28b-his6-SUMO backbone was digested with NdeI and BamHI, treated with Antarctic phosphatase, and the band was extracted from an agarose gel using a kit (Thermo Scientific). The syntenic rceA-rceM genes were amplified together from genomic DNA of the organism with primers prmMRJ064 (5′-ATATAACATATGACGACCATCGTCCC-3′; SEQ ID NO:57) and prmMRJ063 (5′-TTATATGGATCCTCAGGCGGTTTCCCC-3′; SEQ ID NO:58) to add NdeI and BamHI restriction sites to the termini. The PCR product was verified by agarose gel electrophoresis, digested with NdeI and BamHI, and the reaction was cleaned up using a kit (Thermo Scientific). The strM gBlock was amplified with primers prmMRJ062 (5′-ATATAACATATGAGAGCCGCCCCG-3′; SEQ ID NO:59) and prmMRJ063 (5′-TTATATGGATCCTCAGGCGGTTTCCCC-3′; SEQ ID NO:60) to add NdeI and BamHI cut sites on the termini. The PCR product was verified by agarose gel electrophoresis, digested with NdeI and BamHI, and the reaction was cleaned up using a kit (Thermo Scientific). T4 DNA ligase was used to ligate the sticky overhangs into the prepared plasmid backbone. Resultant colonies were screened by colony PCR using primers T7_fw (5′-TAATACGACTCACTATAGGG-3′; SEQ ID NO:47) and T7_rv (5′-GCTAGTTATTGCTCAGCGG-3′; SEQ ID NO:48). Positive hits were sequence verified by ACGT using Sanger sequencing and the same colony PCR primers.

Heterologous expressions were conducted in E. coli cells BL21(DE3). Saturated overnight culture (10 mL) in LB with 50 μg/mL kanamycin was used to inoculate 1 L of TB with 50 μg/mL kanamycin in a 2.5 L baffled Ultra Yield flask (Thomson Scientific). The 1 L culture was incubated in a 37° C. shaker until the OD₆₀₀ reached approximately 0.7, at which time the culture was cold shocked in an ice bath for 30-60 min. After cold shocking, the culture was induced with 200 mM IPTG and placed in a 16° C. shaker for 24 hrs. After 24 hrs, the cells were harvested by centrifugation at 4000×g for 30 min at 4° C., snap frozen in liquid nitrogen, and stored at −80° C. until use.

For protein purification by nickel affinity chromatography, frozen cells were thawed on ice and then resuspended to homogeneity in ice cold lysis buffer (300 mM NaCl, 50 mM sodium phosphate, 20 mM imidazole, 10% glycerol, pH 8.0) with 4 mL of buffer for every 1 g of wet cell mass. After resuspension, lysozyme was then added to a final concentration of 1 mg/mL and incubated on ice for 30 min. After lysozyme treatment, cells were further lysed by sonication. After sonication, lysate was clarified by centrifugation at 15,000×g for 45 min at 4° C. The soluble protein from the clarified supernatant was then batch-bound to nickel-NTA resin (GoldBio) for 60 min on a rotator at 4° C. After binding, resin was added to a 5 mL fritted column, washed with 10 column volumes of lysis buffer, and the protein was eluted in lysis buffer with 250 mM imidazole. For subsequent gel filtration chromatography, protein was concentrated, sterile filtered and loaded onto a HiLoad 16/600 Superdex 200 pg size exclusion column was used at a flow rate of 1 ml/min of lysis buffer without imidazole. Protein was analyzed by SDS-PAGE gel, fractions were pooled and concentrated using Amicon Ultra centrifugal filter columns (MilliporeSigma). Concentrations were measured by Bradford assay and proteins were snap frozen in liquid nitrogen and stored at −80° C. until use. When using frozen protein, all samples were thawed on ice, centrifuged at top speed in a microcentrifuge at 4° C. for 10 min, aggregate removed by transferring supernatant to a fresh tube, and the concentration re-measured.

Briefly, the band corresponding to his6-SonA was extracted from an SDS-PAGE gel, cut into ˜2 mm×2 mm pieces and placed in 1.5 mL LoBind tubes (Eppendorf). Gel cubes were then washed with a 1:1 ratio of 100 mM ammonium bicarbonate (ABC):acetonitrile (ACN) three times until gel pieces appeared clear. After dye removal, they were then dehydrated in 100% ACN until semi-opaque (˜30 sec), and the ACN was subsequently discarded. After rehydration in digest buffer (50 mM ABC and 1:50 units AspN protease (Promega)), gel pieces were placed on ice for 15 min and then were transferred to a 37° C. incubator overnight. The next day, excess liquid from the digest was collected and transferred to a new LoBind tube. Digested peptides were extracted from the gel pieces by first covering them with 60 μL of 50% ACN and 0.3% formic acid (FA) and incubating at room temperature for 15 min. After this incubation, the supernatant was recovered. This extraction was repeated with 60 μL of 80% ACN and 0.3% FA and the supernatant was recovered and placed into the same LoBind tube. The pooled peptide extractions were frozen at −80° C. for 30 min to deactivate the protease. After freezing, the extracted peptides were thawed and dried using a SpeedVac (Eppendorf). Dried peptides were reconstituted in 0.1% FA and purified/desalted using C18 ZipTips according to the manufacturer's instructions. Purified and desalted peptides were again dried using the SpeedVac and then reconstituted in 15-30 μl of 20% ACN, 0.1% FA, and transferred to glass vials for MS analysis. Peptide mass spectrometric analysis (LC-MS/MS HCD) LC-MS/MS measurements of digested peptides was performed as previously described.¹⁶ Briefly, data were obtained on a Thermo Scientific Fusion mass spectrometer furnished with a Dionex Ultimate 3000 UHPLC system with a nLC column (200 mm×75 μm) packed with Vydac 5-μm particles of 300 Å pore size (Hichrom Limited). Elutions used a linear gradient consisting of 0.1% FA in water (solvent A) and 0.1% FA in ACN (solvent B) at a flow rate of 0.3 μl/min. The column was initially equilibrated with 20% solvent B for 5 min and then subjected to a linear increase of solvent B to 85% over 32 min followed by a final elution step of 85% solvent B for 2 min. Mass spectra were acquired in positive-ion mode. Full MS was done at a resolution of 60,000 [automatic gain control (AGC) target, 4×10⁵; maximum ion trap (IT), 50 ms; range, 300 to 1800 m/z], and data-dependent and targeted MS/MS were both performed at a resolution of 15,000 (AGC target, 5×10⁵; maximum IT, 500 ms; isolation window, 2.2) using higher-energy collisional dissociation (HCD). HCD collision energies from 14-20% with steps of ±4% were used during LC-MS/MS measurements.

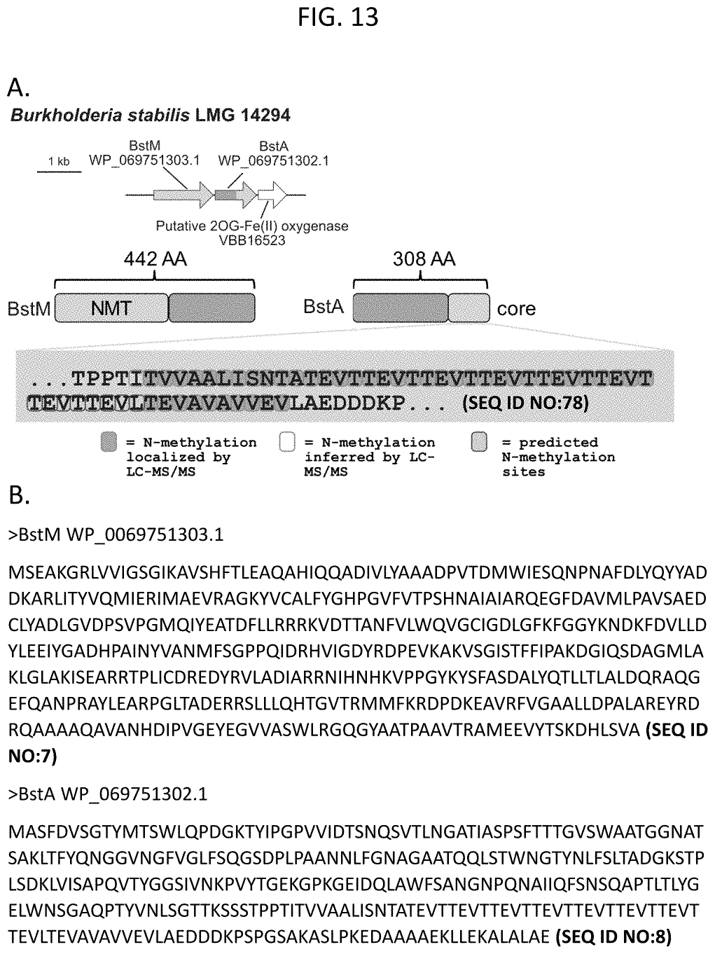

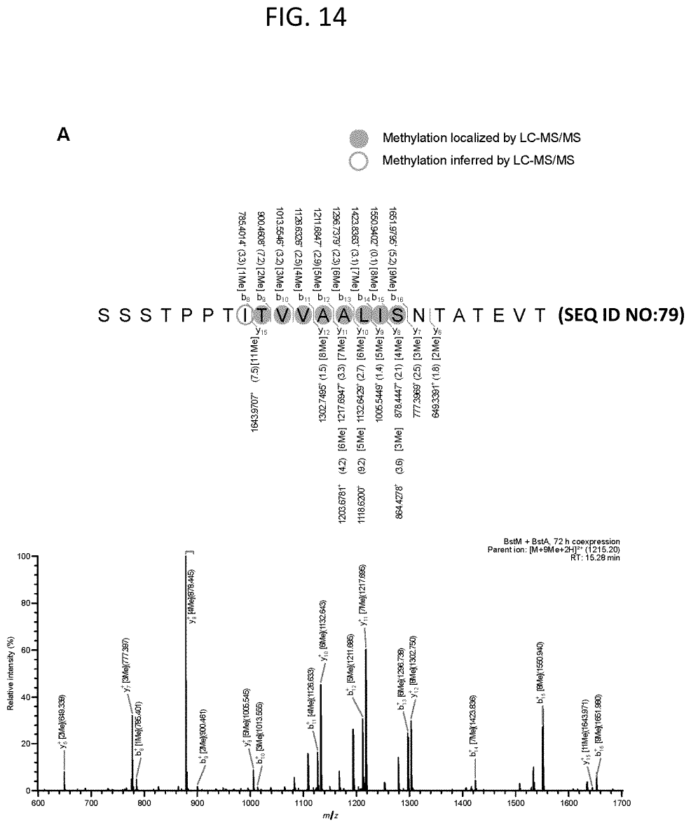

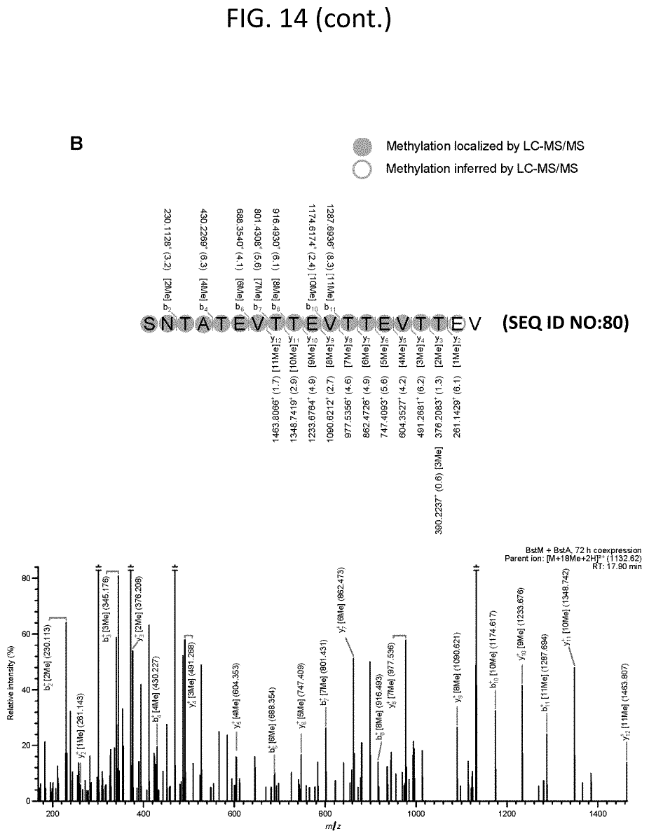

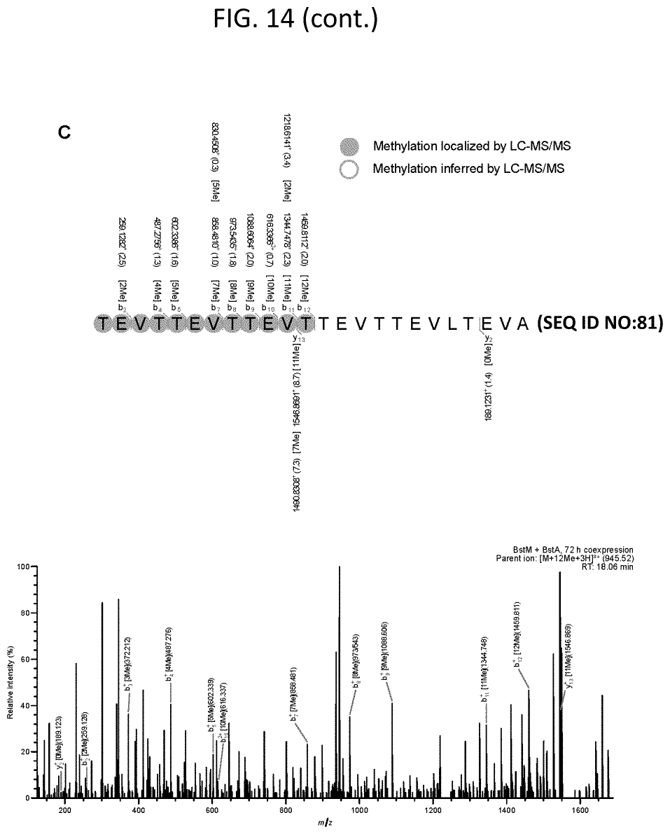

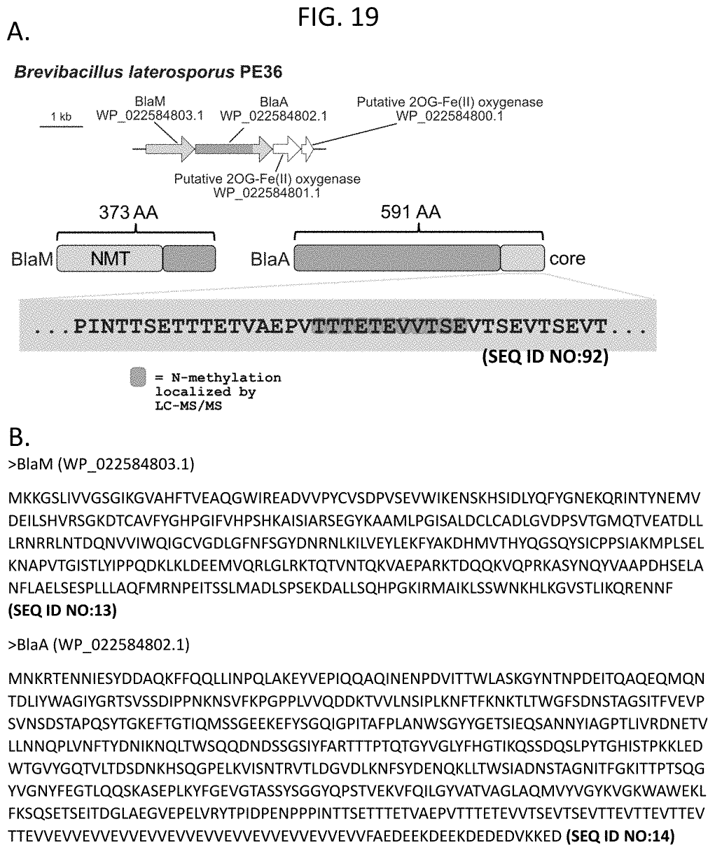

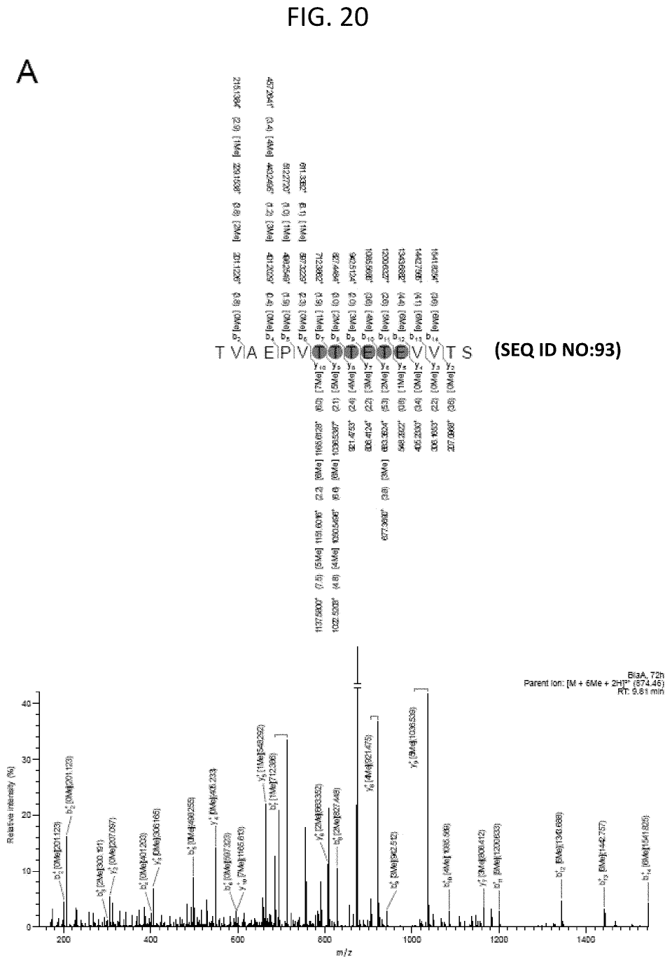

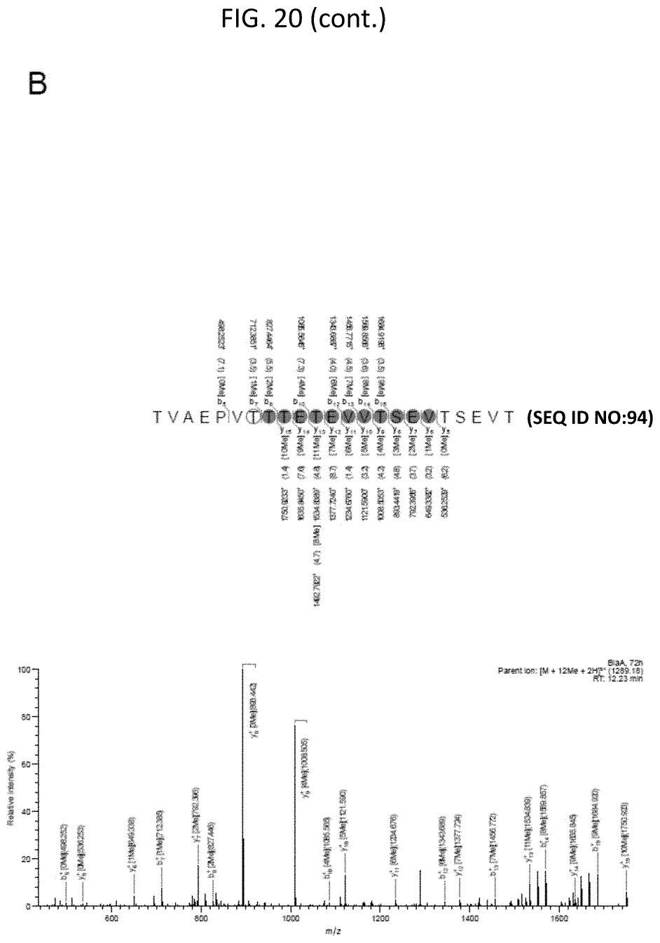

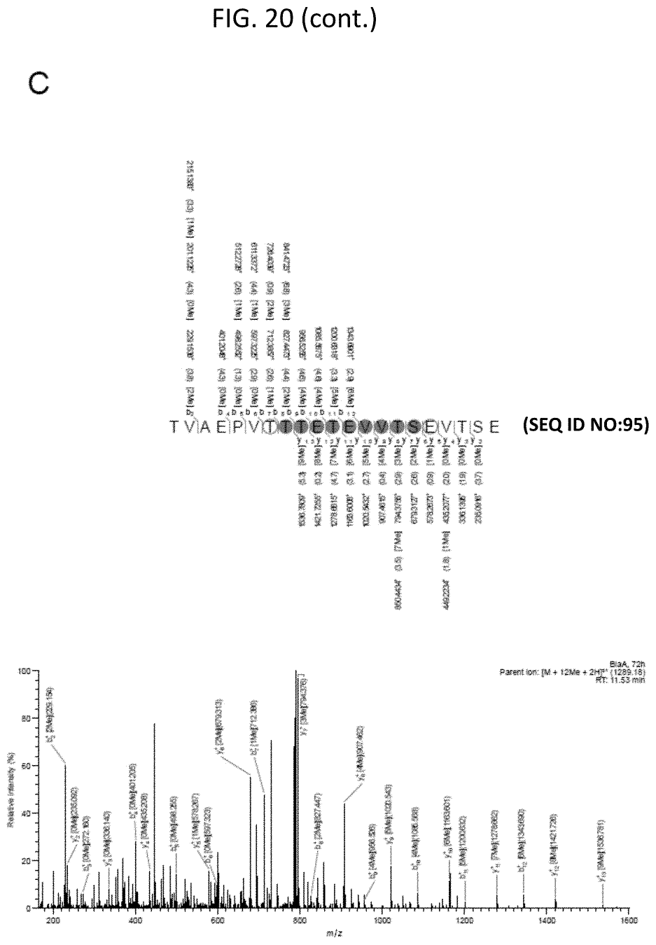

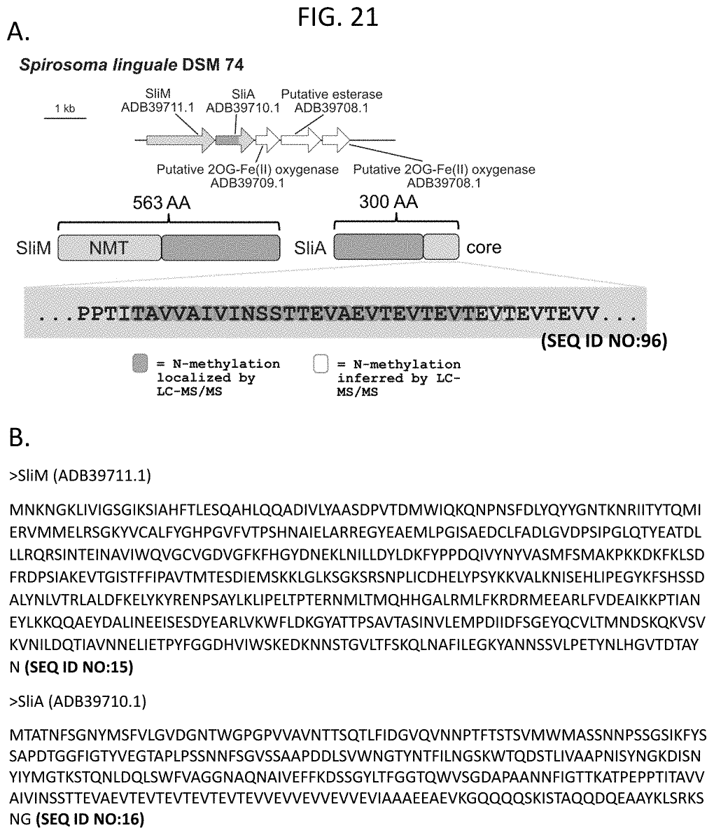

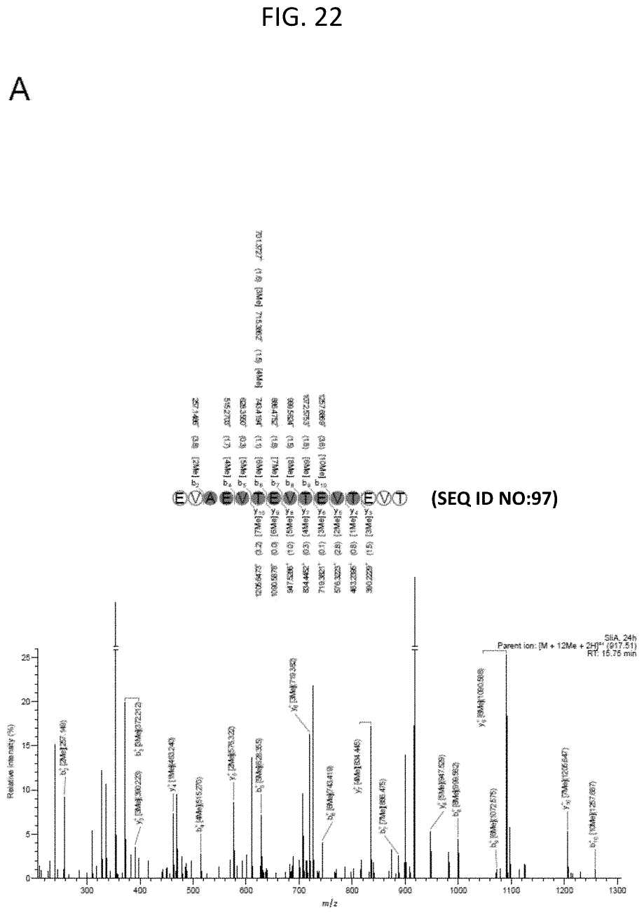

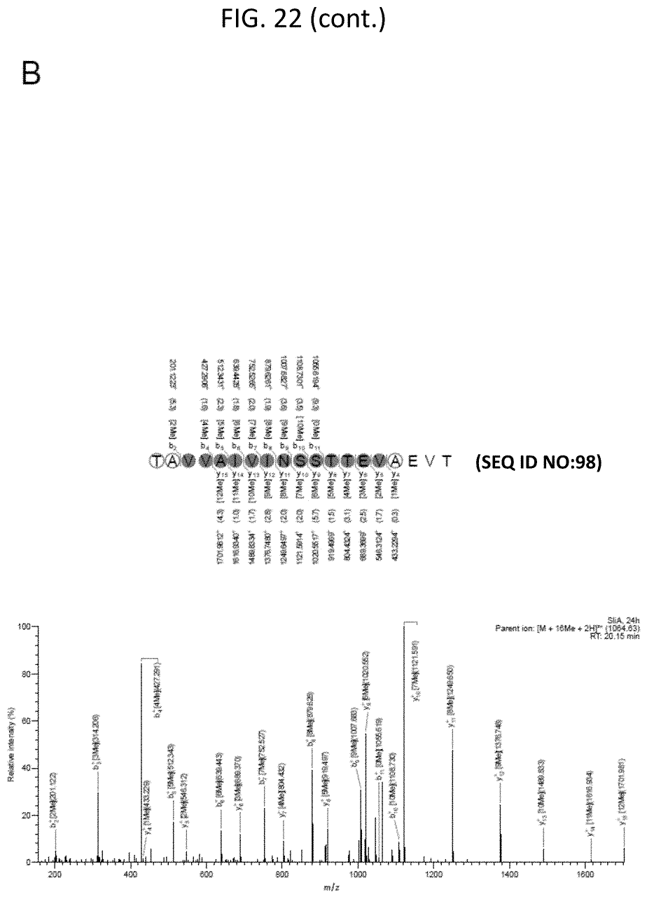

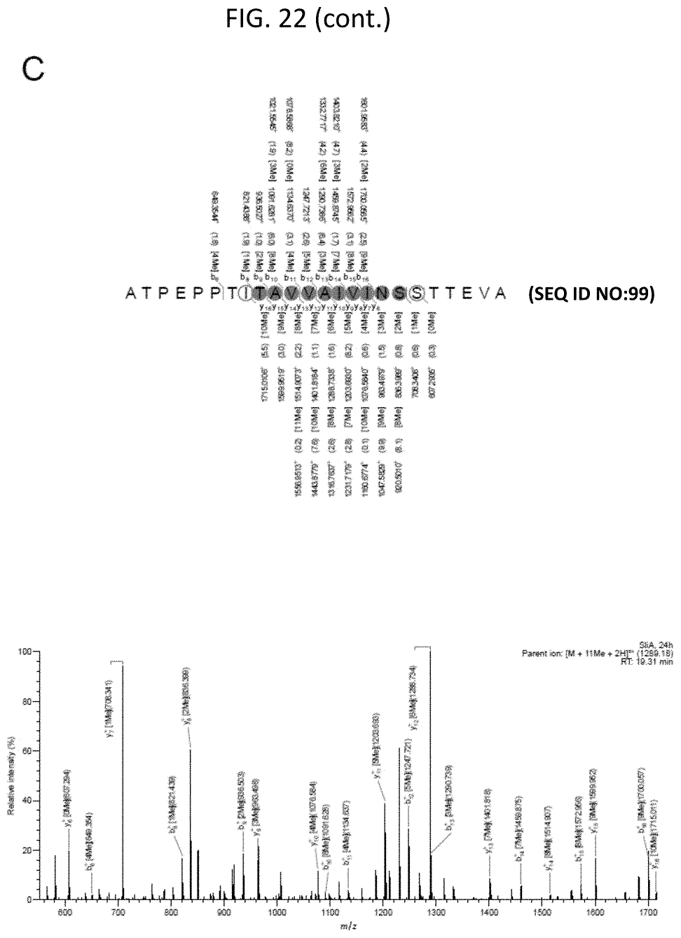

Methods for data described in FIGS. 13, 14, and 19-22.

All constructs for heterologous expression of BstA (WP_069751302.1) and BstM (WP_069751303.1) were made using the genes cloned out of the native organism. N-Terminal histidine and SUMO tags were cloned in front of bstA and bstM. Q5 polymerase was used to amplify genes from genomic DNA according to the manufacturer's protocol. The bstA fragment was amplified using primers F_BstA_pET28HisSumo (5′-CCGGTGGCGCTTCCTTCGATGTGTCCGGAACATACATG-3′; SEQ ID NO:61) and R_BstA_pET28HisSumo (5′-CCGCAAGCTTTCATTCCGCCAGCGCCAGC-3′; SEQ ID NO:62), whereas bstM was amplified using primers F_BstMT_pET28HisSumo (5′-CCGGTGGCAGCGAGGCCAAAGGCAGGC-3′; SEQ ID NO:63) and R_BstMT_pET28HisSumo (5′-GCCGCAAGCTTTCAGGCCACGCTCAGGTGGT-3′; SEQ ID NO:64). Overlap extension PCR was used to combine the SUMO-bstA and SUMO-bstM fragments using primers F_BstA_coexp_HindIII (5′-GCGGAAGCTTAATACGACTCACTATAGGGGAATTGTGAGCG-3′; SEQ ID NO:65) and R_BstA_coexp_XhoI (5′-AATCCTCGAGTCAGGCCACGCTCAGGTGG-3′; SEQ ID NO:66) that added recognition sequences for the restriction endonucleases HindIII and XhoI (New England Biolabs). Compatible sticky-ends were produced with the pET28 vector backbone digested with the same endonucleaes. T4 Ligase (New England Biolabs) was used to ligate the backbone and insert together, before transformation into electrocompetent TOP10 E. coli cells.

Genes were expressed in E. coli BL21(DE3) cells and induced with 0.2 mM IPTG final concentration at 16° C. for 72 h. Cells were harvested by centrifugation at 4,000×g for 35 minutes, and lysed by sonication. After centrifugation at 15,000×g for 45 minutes, the lysis supernatant was transferred to a fresh tube. Recombinant proteins were purified from the lysis supernatant via nickel-chelate chromatography based on manufacturers' recommendations (Ni-NTA, Gold Biotechnology).

Proteolytic digestions and MS analysis: Proteins were digested using an in-gel digestion method. Appropriate bands from soluble fractions were excised from SDS-PAGE gels and cut into ˜2 mm×2 mm cubes. Gel pieces were washed with a 1:1 ratio of 100 mM ammonium bicarbonate (ABC):acetonitrile (ACN) three times until all the dye was removed. Gel pieces were then dehydrated in 100% ACN until semi-opaque (˜30 sec), after which the ACN was discarded. Gel pieces were then rehydrated in digestion buffer (50 mM ABC, 5 mM CaCl2) with a 1:4 molar ratio of proteinase K (protease):analyte protein. Enough digestion buffer was added so the gel pieces were completely submerged, before overnight incubation at 37° C. Digestion supernatant was recovered the next day, and placed in a fresh tube. Digested peptides were recovered by dehydrating the gel pieces in two successive steps. First, 60 μL of 50% ACN and 0.3% formic acid (FA) was added, incubated for 15 min at room temperature and recovered. Second, 60 μL of 80% ACN and 0.3% FA was added, incubated and recovered. The extracted peptides were pooled and frozen at −80° C. for 30 min to deactivate the protease. Peptide solutions were then thawed and dried using a SpeedVac (Eppendorf). Peptides were resuspended in 0.1% FA and further purified and desalted using C18 ZipTips according to the manufacturer's specifications. After drying the samples again, peptides were resuspended in 30 μl of 20% ACN, 0.1% FA, and transferred to glass vials for MS analysis.

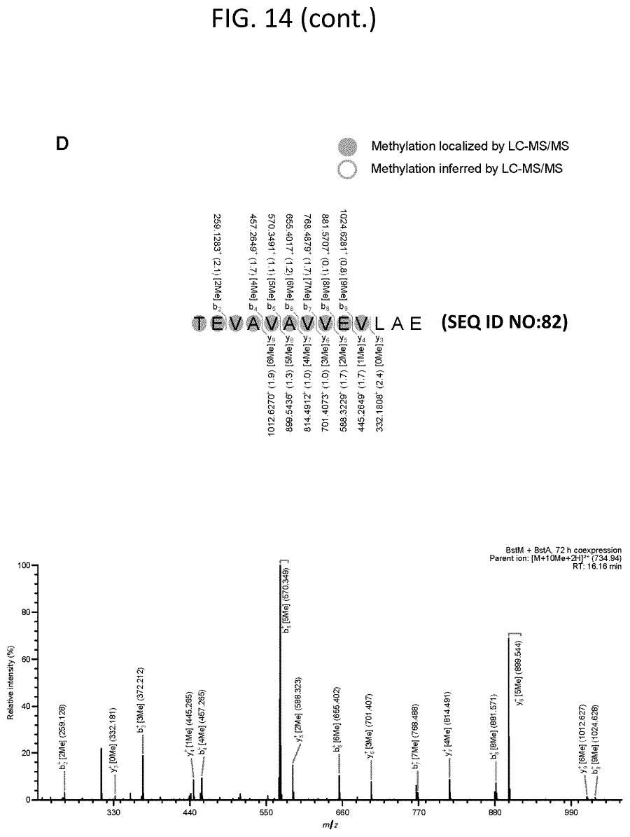

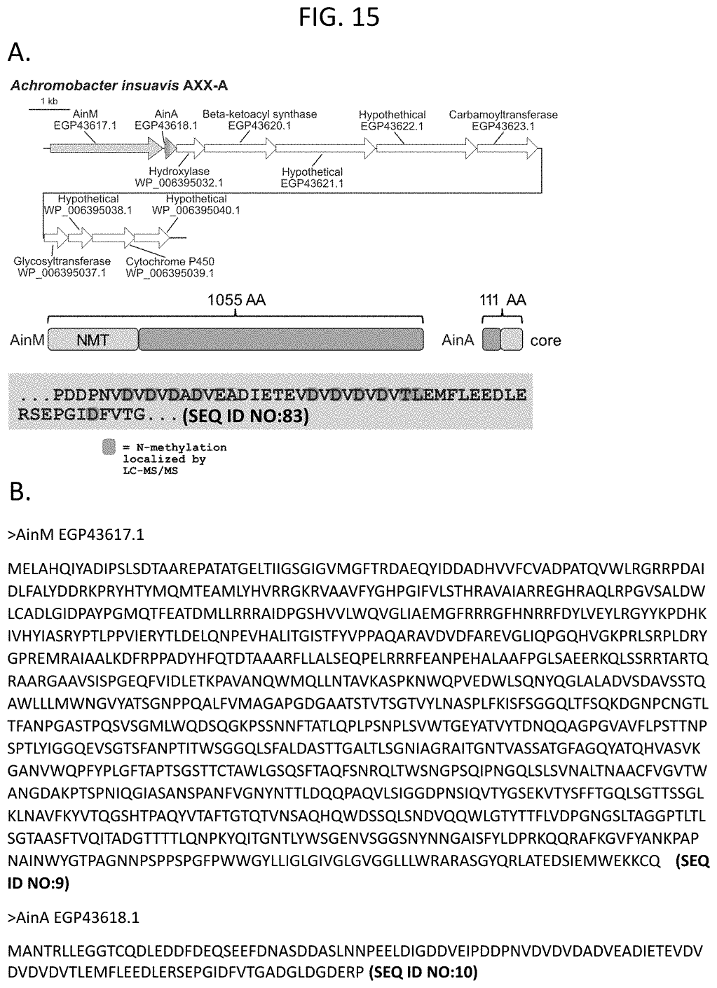

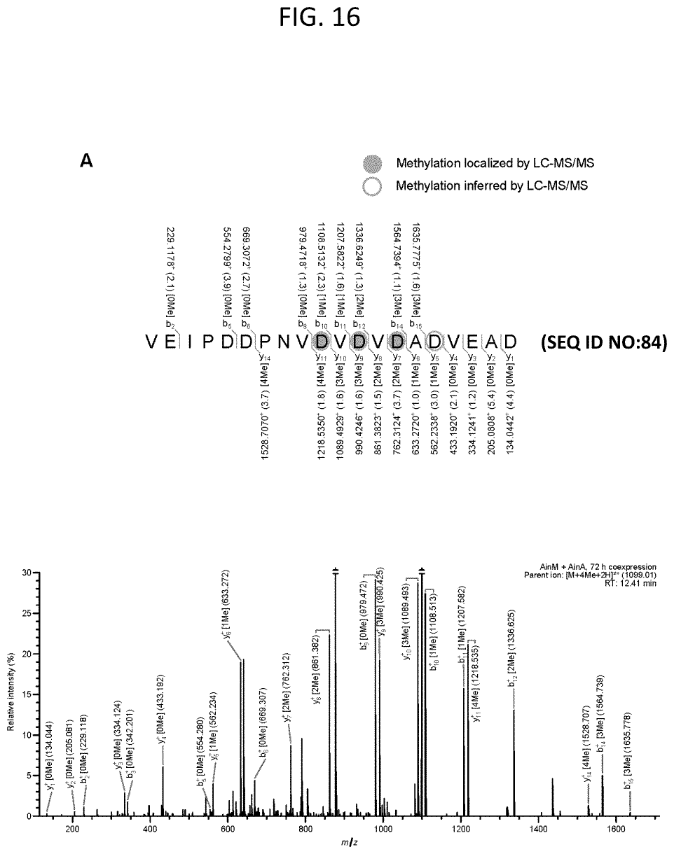

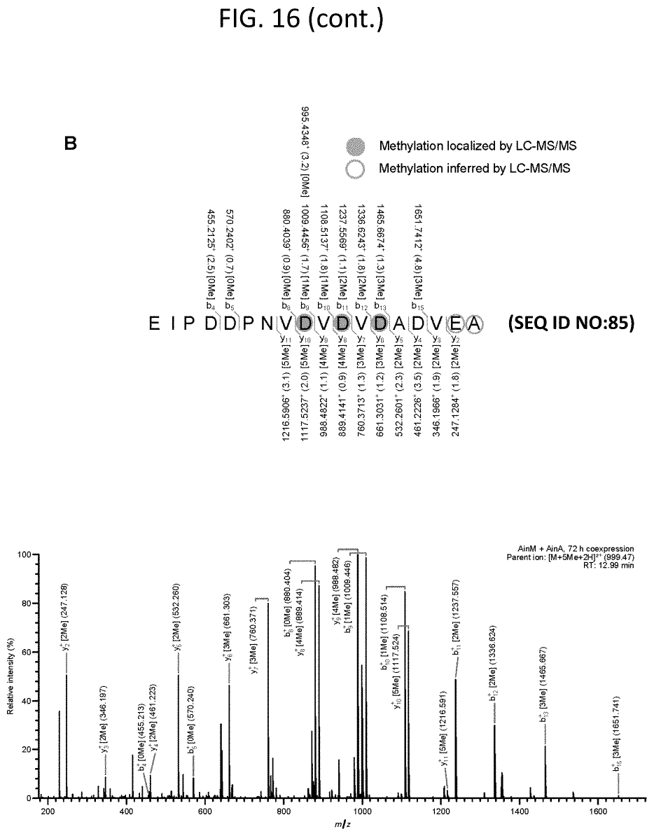

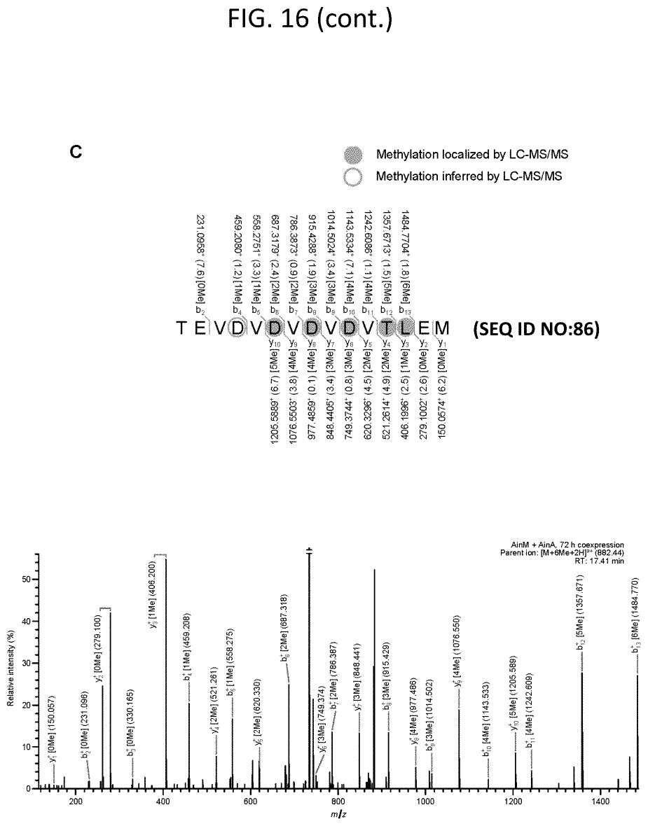

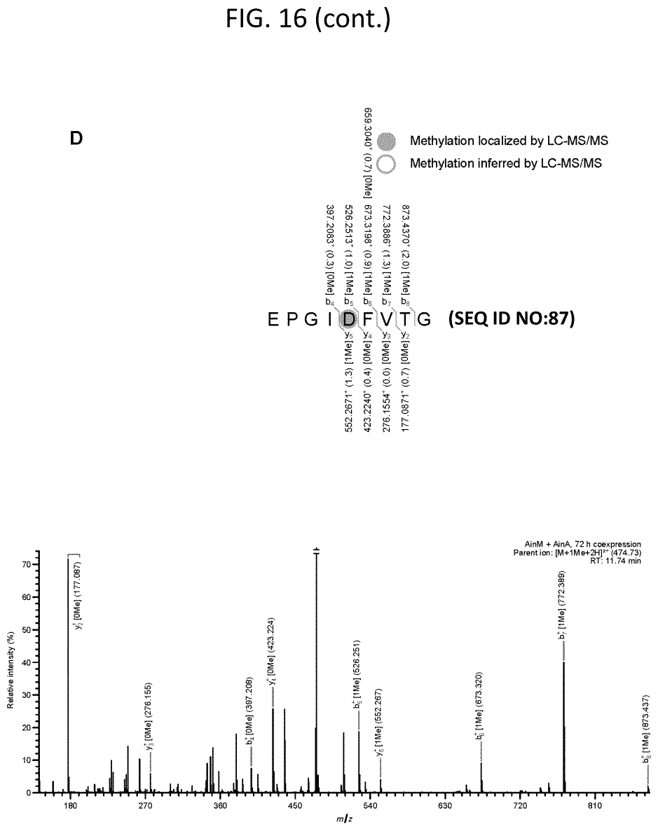

Methods for Data Described in FIGS. 15 and 16.

Genes were expressed in E. coli BL21(DE3) cells and induced with 0.2 mM IPTG final concentration at 16° C. for 72 h. Cells were harvested by centrifugation at 4,000×g for 35 minutes, and lysed by sonication. After centrifugation at 15,000×g for 45 minutes, the lysis supernatant was transferred to a fresh tube. Recombinant proteins were purified from the lysis supernatant via nickel-chelate chromatography based on manufacturers' recommendations (Ni-NTA, Gold Biotechnology).

Proteins were digested using an in-gel digestion method. Appropriate bands from soluble fractions were excised from SDS-PAGE gels and cut into ˜2 mm×2 mm cubes. Gel pieces were washed with a 1:1 ratio of 100 mM ammonium bicarbonate (ABC):acetonitrile (ACN) three times until all the dye was removed. Gel pieces were then dehydrated in 100% ACN until semi-opaque (˜30 sec), after which the ACN was discarded. Gel pieces were then rehydrated in digestion buffer (50 mM ABC, 5 mM CaCl2) with a 1:4 molar ratio of Proteinase K (protease):analyte protein. Enough digestion buffer was added so the gel pieces were completely submerged, before overnight incubation at 37° C. Digestion supernatant was recovered the next day, and placed in a fresh tube. Digested peptides were recovered by dehydrating the gel pieces in two successive steps. First, 60 μL of 50% ACN and 0.3% formic acid (FA) was added, incubated for 15 min at room temperature and recovered. Second, 60 μL of 80% ACN and 0.3% FA was added, incubated and recovered. The extracted peptides were pooled and frozen at −80° C. for 30 min to deactivate the protease. Peptide solutions were then thawed and dried using a SpeedVac (Eppendorf). Peptides were resuspended in 0.1% FA and further purified and desalted using C18 ZipTips according to the manufacturer's specifications. After drying the samples again, peptides were resuspended in 30 μl of 20% ACN, 0.1% FA, and transferred to glass vials for MS analysis.

LC-MS/MS data was recorded on a Thermo Scientific Fusion mass spectrometer equipped with a Dionex Ultimate 3000 UHPLC system using a nLC column (200 mm×75 μm) packed using Vydac 5-μm particles with a 300 Å pore size (Hichrom Limited). Elution was performed with a linear gradient using water with 0.1% FA (solvent A) and ACN with 0.1% FA (solvent B) at a flow rate of 0.3 μl/min. The column was equilibrated with 20% solvent B for 5 min, followed by a linear increase of solvent B to 85% over 32 min and a final elution step with 85% solvent B for 2 min. Mass spectra were acquired in positive-ion mode. Full MS was done at a resolution of 60,000 [automatic gain control (AGC) target, 4×105; maximum ion trap (IT), 50 ms; range, 300 to 1800 m/z], and data-dependent as well as targeted MS/MS was performed at a resolution of 15,000 (AGC target, 5×105; maximum IT, 500 ms; isolation window, 2.2) using higher-energy collisional dissociation (HCD). HCD collision energy of 15% with steps of ±3% were used. Data were processed using Thermo Fisher Xcalibur software and MaxQuant.

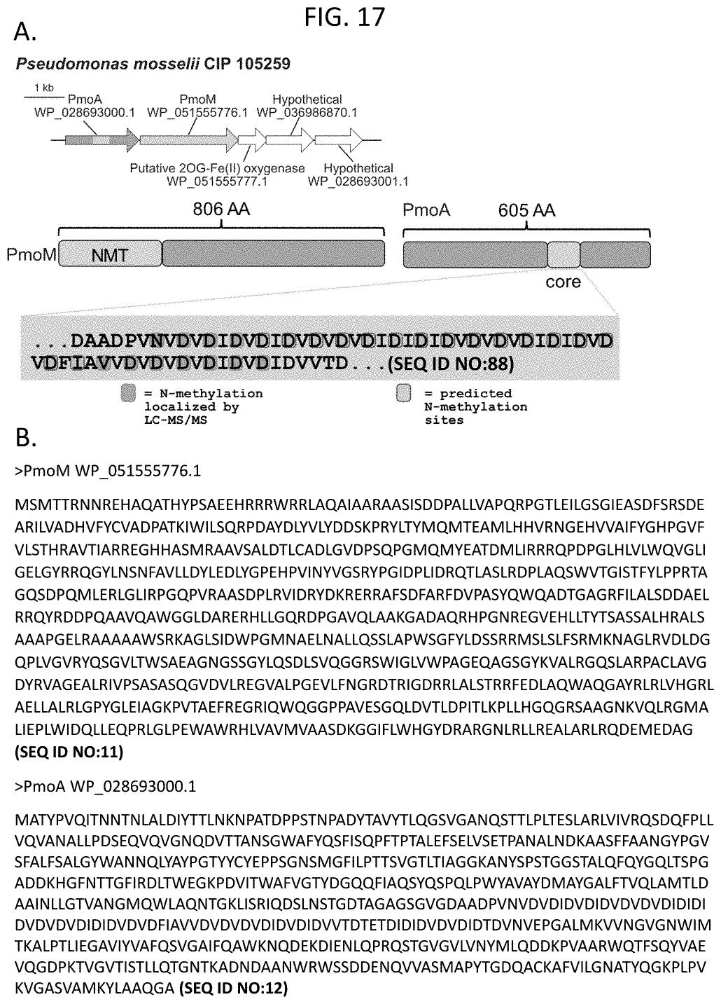

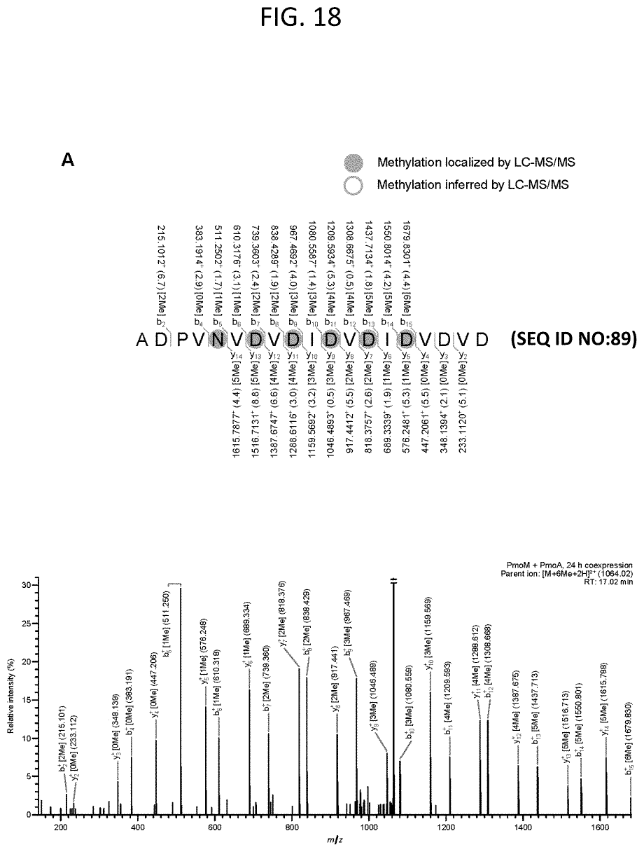

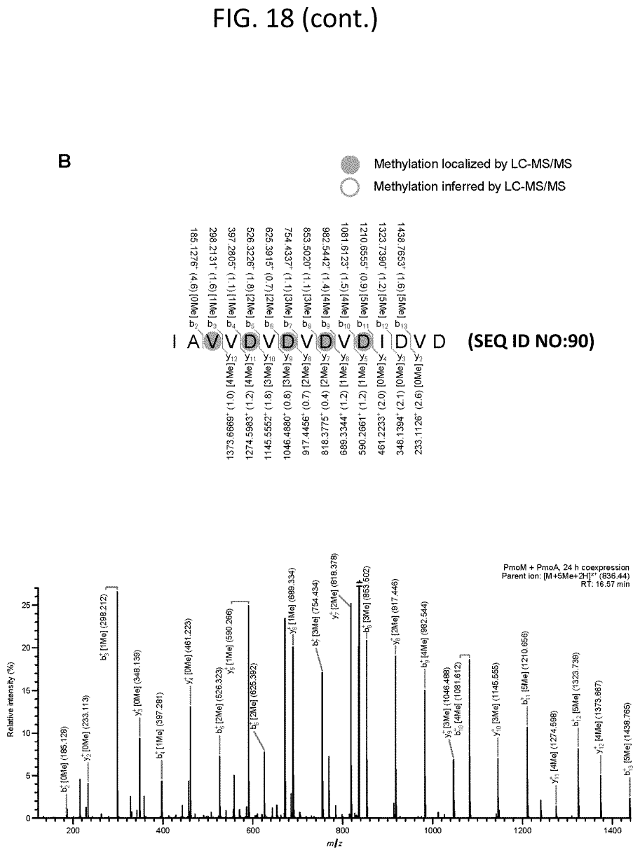

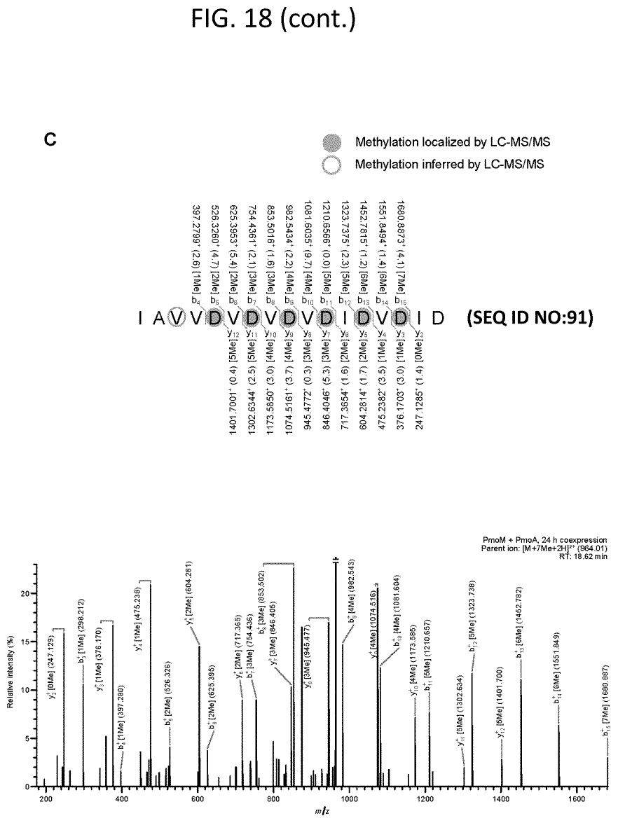

Methods for Data Described in FIGS. 17 and 18.

All constructs for heterologous expression of PmoA (WP_028693000.1) and PmoM (WP_051555776.1) were made using the genes cloned out of the native organism. An N-terminal histidine tag was cloned in front of pmoA. Q5 polymerase was used to amplify pmoA and pmoM genes as a single operon with primers Fwd_pET28b_NHis_Pmosselii_Repeat_RiPP (5′-GCTCGAGTGCGGCCGCAAGCTTGCATGTTCATCCGGCATCCTCCATTTCATCCT-3′; SEQ ID NO:67) and R_Pmosselii_NHisRepRiPP_HindIII (5′-GCTCGAGTGCGGCCGCAAGCTTGCATGTTCATCCGGCATCCTCCATTTCATCCT-3′; SEQ ID NO:68) from the extracted genomic DNA according to the manufacturer's protocol. The pET28 backbone was amplified using primers Rev_pET28b_NdeI_Backbone_Gibson (5′-CATATGGCTGCCGCGCGGCACCA-3′; SEQ ID NO: 69) and F_backbone_pET28NHisSumoPmoA (5′-GCGCCTGAAAGCTTGCGGCCGCACTCGA-3′; SEQ ID NO:70). The constructs was assembled using HiFi DNA Assembly Master Mix (New England Biolabs) before transformation into electrocompetent TOP10 E. coli cells.

Genes were expressed in E. coli BL21(DE3) cells and induced with 0.2 mM IPTG final concentration at 16° C. for 72 h. Cells were harvested by centrifugation at 4,000×g for 35 minutes, and lysed by sonication. After centrifugation at 15,000×g for 45 minutes, the lysis supernatant was transferred to a fresh tube. Recombinant proteins were purified from the lysis supernatant via nickel-chelate chromatography based on manufacturers' recommendations (Ni-NTA, Gold Biotechnology).

Proteins were digested using an in-gel digestion method. Appropriate bands from soluble fractions were excised from SDS-PAGE gels and cut into ˜2 mm×2 mm cubes. Gel pieces were washed with a 1:1 ratio of 100 mM ammonium bicarbonate (ABC):acetonitrile (ACN) three times until all the dye was removed. Gel pieces were then dehydrated in 100% ACN until semi-opaque (˜30 sec), after which the ACN was discarded. Gel pieces were then rehydrated in digestion buffer (50 mM ABC, 5 mM CaCl2) with a 1:4 molar ratio of Proteinase K (protease):analyte protein. Enough digestion buffer was added so the gel pieces were completely submerged, before overnight incubation at 37° C. Digestion supernatant was recovered the next day, and placed in a fresh tube. Digested peptides were recovered by dehydrating the gel pieces in two successive steps. First, 60 μL of 50% ACN and 0.3% formic acid (FA) was added, incubated for 15 min at room temperature and recovered. Second, 60 μL of 80% ACN and 0.3% FA was added, incubated and recovered. The extracted peptides were pooled and frozen at −80° C. for 30 min to deactivate the protease. Peptide solutions were then thawed and dried using a SpeedVac (Eppendorf). Peptides were resuspended in 0.1% FA and further purified and desalted using C18 ZipTips according to the manufacturer's specifications. After drying the samples again, peptides were resuspended in 30 μl of 20% ACN, 0.1% FA, and transferred to glass vials for MS analysis.

LC-MS/MS data was recorded on a Thermo Scientific Fusion mass spectrometer equipped with a Dionex Ultimate 3000 UHPLC system using a nLC column (200 mm×75 μm) packed using Vydac 5-μm particles with a 300 Å pore size (Hichrom Limited). Elution was performed with a linear gradient using water with 0.1% FA (solvent A) and ACN with 0.1% FA (solvent B) at a flow rate of 0.3 μl/min. The column was equilibrated with 20% solvent B for 5 min, followed by a linear increase of solvent B to 85% over 32 min and a final elution step with 85% solvent B for 2 min. Mass spectra were acquired in positive-ion mode. Full MS was done at a resolution of 60,000 [automatic gain control (AGC) target, 4×10⁵; maximum ion trap (IT), 50 ms; range, 300 to 1800 m/z], and data-dependent as well as targeted MS/MS was performed at a resolution of 15,000 (AGC target, 5×10⁵; maximum IT, 500 ms; isolation window, 2.2) using higher-energy collisional dissociation (HCD). HCD collision energy of 16% with steps of ±3% were used. Data were processed using Thermo Fisher Xcalibur software and MaxQuant.

### REFERENCES

(1) Biron, E.; Chatterjee, J.; Ovadia, O.; Langenegger, D.; Brueggen, J.; Hoyer, D.; Schmid, H. A.; Jelinek, R.; Gilon, C.; Hoffman, A.; et al. Improving Oral Bioavailability of Peptides by Multiple N-Methylation: Somatostatin Analogues. Angew. Chem. Int. Ed. Engl. 2008, 47 (14), 2595-2599. https://doi.org/10.1002/anie.200705797.
(2) Beck, J. G.; Chatterjee, J.; Laufer, B.; Kiran, M. U.; Frank, A. O.; Neubauer, S.; Ovadia, O.; Greenberg, S.; Gilon, C.; Hoffman, A.; et al. Intestinal Permeability of Cyclic Peptides: Common Key Backbone Motifs Identified. J. Am. Chem. Soc. 2012, 134 (29), 12125-12133. https://doi.org/10.1021/ja303200d.
(3) Nandel, F. S.; Jaswal, R. R. Conformational Study of N-Methylated Alanine Peptides and Design of AP Inhibitor. Indian J. Biochem. Biophys. 2014, 51 (1), 7-18.
(4) Chatterjee, J.; Gilon, C.; Hoffman, A.; Kessler, H. N-Methylation of Peptides: A New Perspective in Medicinal Chemistry. Acc. Chem. Res. 2008, 41 (10), 1331-1342. https://doi.org/10.1021/ar8000603.
(5) Niquille, D. L.; Hansen, D. A.; Mori, T.; Fercher, D.; Kries, H.; Hilvert, D. Nonribosomal Biosynthesis of Backbone-Modified Peptides. Nat. Chem. 2018, 10 (3), 282-287. https://doi.org/10.1038/nchem.2891.
(6) Velkov, T.; Horne, J.; Scanlon, M. J.; Capuano, B.; Yuriev, E.; Lawen, A. Characterization of the N-Methyltransferase Activities of the Multifunctional Polypeptide Cyclosporin Synthetase. Chem. Biol. 2011, 18 (4), 464-475. https://doi.org/10.1016/j.chembiol.2011.01.017.
(7) Arnison, P. G.; Bibb, M. J.; Bierbaum, G.; Bowers, A. A.; Bugni, T. S.; Bulaj, G.; Camarero, J. A.; Campopiano, D. J.; Challis, G. L.; Clardy, J.; et al. Ribosomally Synthesized and Post-Translationally Modified Peptide Natural Products: Overview and Recommendations for a Universal Nomenclature. Nat. Prod. Rep. 2013, 30 (1), 108-160. https://doi.org/10.1039/c2np20085f.
(8) Mayer, A.; Anke, H.; Sterner, O. Omphalotin, a New Cyclic Peptide with Potent Nematicidal Activity from Omphalotus olearius: I. Fermentation and Biological Activity. Nat. Prod. Lett. 1997, 10 (1), 25-32. https://doi.org/10.1080/10575639708043691.
(9) BUchel, E.; Martini, U.; Mayer, A.; Anke, H.; Sterner, O. Omphalotins B, C and D, Nematicidal Cyclopeptides from Omphalotus olearius. Absolute Configuration of Omphalotin A. Tetrahedron 1998, 54 (20), 5345-5352. https://doi.org/10.1016/50040-4020(98)00209-9.
(10) Liermann, J. C.; Opatz, T.; Kolshorn, H.; Antelo, L.; Hof, C.; Anke, H. Omphalotins E-I, Five Oxidatively Modified Nematicidal Cyclopeptides from Omphalotus olearius. Eur. J. Org. Chem. 2009, 2009 (8), 1256-1262. https://doi.org/10.1002/ejoc.200801068.
(11) Ványolós, A.; Dékány, M.; Kovács, B.; Krámos, B.; Bérdi, P.; Zupkó, I.; Hohmann, J.; Béni, Z. Gymnopeptides A and B, Cyclic Octadecapeptides from the Mushroom Gymnopus Fusipes. Org. Lett. 2016, 18 (11), 2688-2691. https://doi.org/10.1021/acs.orglett.6b01158.
(12) Kries, H. Biosynthetic Engineering of Nonribosomal Peptide Synthetases. J. Pept. Sci. 2016, 22 (9), 564-570. https://doi.org/10.1002/psc.2907.
(13) Hudson, G. A.; Mitchell, D. A. RiPP Antibiotics: Biosynthesis and Engineering Potential. Curr. Opin. Microbiol. 2018, 45, 61-69. https://doi.org/10.1016/j.mib.2018.02.010.
(14) van der Velden, N. S.; Kahn, N.; Helf, M. J.; Piel, J.; Freeman, M. F.; Kunzler, M. Autocatalytic Backbone N-Methylation in a Family of Ribosomal Peptide Natural Products. Nat. Chem. Biol. 2017, 13 (8), 833-835. https://doi.org/10.1038/nchembio.2393.
(15) Ramm, S.; Krawczyk, B.; Mühlenweg, A.; Poch, A.; Mösker, E.; Süssmuth, R. D. A
Self-Sacrificing N-Methyltransferase Is the Precursor of the Fungal Natural Product Omphalotin. Angew. Chem. Int. Ed. Engl. 2017, 56 (33), 9994-9997. https://doi.org/10.1002/anie.201703488.
(16) Quijano, M. R.; Zach, C.; Miller, F. S.; Lee, A. R.; Imani, A. S.; Künzler, M.; Freeman, M. F. Distinct Autocatalytic α-N-Methylating Precursors Expand the Borosin RiPP Family of Peptide Natural Products. J. Am. Chem. Soc. 2019, 141 (24), 9637-9644. https://doi.org/10.1021/jacs.9b03690.
(17) Song, H.; van der Velden, N. S.; Shiran, S. L.; Bleiziffer, P.; Zach, C.; Sieber, R.; Imani, A. S.; Krausbeck, F.; Aebi, M.; Freeman, M. F.; et al. A Molecular Mechanism for the Enzymatic Methylation of Nitrogen Atoms within Peptide Bonds. Sci. Adv. 2018, 4 (8), eaat2720. https://doi.org/10.1126/sciadv.aat2720.
(18) Künzler, M.; van der Velden, N. S.; Freeman, M. F.; Piel, J.; Aebi, M.; Kahn, N. Novel Multiply Backbone N-Methyl Transferases and Uses Thereof. WO2017EP58327, 2017.
(19) Aebi, M.; Künzler, M.; Piel, J.; Freeman, M. F.; van der Velden, N. S.; Kahn, N. Novel Multiply Backbone N-Methyl Transferases and Uses Thereof. US20190112583A1, 2019.
(20) Andrade, M. A.; Perez-Iratxeta, C.; Ponting, C. P. Protein Repeats: Structures, Functions, and Evolution. J. Struct. Biol. 2001, 134 (2-3), 117-131. https://doi.org/10.1006/jsbi.2001.4392.
(21) Chou, S.-H.; Galperin, M. Y. Diversity of Cyclic Di-GMP-Binding Proteins and Mechanisms. J. Bacteriol. 2016, 198 (1), 32-46. https://doi.org/10.1128/JB.00333-15.
(22) Huelsenbeck, J. P.; Ronquist, F. MRBAYES: Bayesian Inference of Phylogenetic Trees. Bioinformatics 2001, 17 (8), 754-755. https://doi.org/10.1093/bioinformatics/17.8.754.
(23) Kitagawa, M.; Ara, T.; Arifuzzaman, M.; loka-Nakamichi, T.; Inamoto, E.; Toyonaga, H.; Mori, H. Complete Set of ORF Clones of Escherichia Coli ASKA Library (A Complete Set of E. Coli K-12 ORF Archive): Unique Resources for Biological Research. DNA Res. 2006, 12 (5), 291-299. https://doi.org/10.1093/dnares/dsi012.
(24) Kamat, S. S.; Bagaria, A.; Kumaran, D.; Holmes-Hampton, G. P.; Fan, H.; Sali, A.; Sauder, J. M.; Burley, S. K.; Lindahl, P. A.; Swaminathan, S.; et al. Catalytic Mechanism and Three-Dimensional Structure of Adenine Deaminase. Biochemistry 2011, 50 (11), 1917-1927. https://doi.org/10.1021/bi101788n.
(25) Duchin, S.; Vershinin, Z.; Levy, D.; Aharoni, A. A Continuous Kinetic Assay for Protein and DNA Methyltransferase Enzymatic Activities. Epigenetics Chromatin 2015, 8 (1), 56. https://doi.org/10.1186/513072-015-0048-y.

The present invention has been described in terms of one or more preferred embodiments, and it should be appreciated that many equivalents, alternatives, variations, and modifications, aside from those expressly stated, are possible and within the scope of the invention.

1. A method for producing an alpha-N-methylated target peptide, the method comprising:
(a) contacting a split borosin alpha-N-methyltransferase protein to a target peptide; and
(b) incubating the split borosin alpha-N-methyltransferase protein and the target peptide in the presence of a methyl donor to produce an alpha-N-methylated target peptide.

2. The method of claim 1, wherein the split borosin alpha-N-methyltransferase comprises an amino acid sequence having at least 70% sequence similarity to an amino acid sequence selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15.

3. (canceled)

4. The method of claim 1, wherein the split borosin alpha-N-methyltransferase comprises an amino acid sequence selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15.

5. The method of claim 1, wherein the target peptide is a split borosin precursor comprising an amino acid sequence having at least 70% sequence similarity to an amino acid sequence selected from SEQ ID NO:2, SEQ ID NO:4, SEQ ID NO:6, SEQ ID NO:8, SEQ ID NO:10, SEQ ID NO:12, SEQ ID NO:14, and SEQ ID NO:16.

6. (canceled)

7. The method of claim 1, wherein the method is in vitro and the split borosin alpha-N-methyltransferase and target peptide are isolated proteins.

8. The method of claim 1, wherein one or more of the split borosin alpha-N-methyltransferase and target peptide is a recombinant protein or synthetic protein.

9. The method of claim 1, wherein the isolated split borosin methyltransferase protein is obtained by:
(i) introducing into a cell an exogenous expression vector comprising a nucleotide sequence encoding a split borosin methyltransferase protein;
(ii) expressing the split borosin methyltransferase protein in the cell; and
(iii) purifying the expressed split borosin methyltransferase protein.

10. The method of claim 1, wherein the method is in vivo and contacting of the split borosin alpha-N-methyltransferase to the target peptide occurs in a host cell.

11. The method of claim 10, the method further comprising introducing into a cell one or more expression vectors encoding the split borosin methyltransferase protein and the target peptide, optionally wherein the expression vector is a plasmid.

12. The method of claim 1, wherein the split borosin alpha-N-methyltransferase comprises an affinity tag and/or a solubility tag.

13-14. (canceled)

15. The method of claim 1, wherein the method further comprises isolating the N-methylated target peptide.

16. An in vivo method of producing a peptide library comprising random alpha-N-methylated peptides, the method comprising:
(a) introducing into a cell one or more expression vectors comprising a nucleotide sequence encoding a split borosin alpha-N-methyltransferase and one or more nucleotide sequences encoding one or more split borosin precursors to produce alpha N-methylated peptides, wherein the one or more nucleotide sequences encoding the one or more split borosin precursors comprise one or more genetic variation relative to a nucleotide sequence encoding a wild-type split borosin precursor; and
(b) isolating the alpha-N-methylated peptides to produce the peptide library.

17. The method of claim 16, the method further comprising:
detecting production of alpha N-methylated peptides prior to isolation in step (b).

18. The method of claim 16, wherein the method comprises introducing one or more genetic variations by random mutagenesis or site-directed mutagenesis.

19. (canceled)

20. The method of claim 16, wherein the split borosin alpha-N-methyltransferase polypeptide is expressed in cis or trans to the one or more split borosin precursor polypeptides.

21. (canceled)

22. The method of claim 16, wherein the split borosin alpha-N-methyltransferase comprises an amino acid sequence having at least 70% sequence similarity to an amino acid sequence selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15.

23. The method of claim 16, wherein the split borosin alpha-N-methyltransferase comprises an amino acid sequence selected from SEQ ID NO:1, SEQ ID NO:3, SEQ ID NO:5, SEQ ID NO:7, SEQ ID NO:9, SEQ ID NO:11, SEQ ID NO:13, and SEQ ID NO:15.

24-25. (canceled)

26. The method of claim 16, wherein one or more of the split borosin alpha-N-methyltransferase and the one or more split borosin precursors comprise an affinity tag and/or a solubility tag.

27-28. (canceled)

29. An in vitro method of producing a peptide library comprising random alpha-N-methylated peptides, the method comprising:
(a) contacting an isolated split borosin alpha-N-methyltransferase to one or more split borosin precursors comprising one or more genetic variations relative to a wild-type split borosin precursor in the presence of a methyl donor to produce one or more alpha-N-methylated split borosin precursors; and
(b) isolating the alpha-N-methylated split borosin precursor peptides to produce the peptide library.

30. The method of claim 29, wherein the method comprises detecting production of alpha-N-methylated split precursor peptides prior to isolation in step (b).

31. The method of claim 29 or 30, wherein the one or more split borosin precursors are obtained by:
(i) introducing into a cell an expression vector comprising one or more nucleotide sequences encoding one or more split borosin precursors comprising one or more genetic variations relative to a nucleotide sequence encoding a wild-type split borosin precursor;
(ii) expressing the one or more split borosin precursors in the cell; and
(iii) purifying the expressed one or more split borosin precursor.

32. The method of claim 31, wherein the method comprises introducing one or more genetic variations by random mutagenesis or site-directed mutagenesis.

33-38. (canceled)


SEQUENCE LISTING (229 sequences)

SEQ ID NO: 1 - protein, 263 aa, Shewanella oneidensis
MGSLVCVGTGLQLAGQISVLSRSYIEHADIVFSLLPDGFSQRWLTKLNPNVINLQQFYAQNGEVKNRRDTYEQMVNAILDAVRAGKKTVCALYGHPGVFACVSHMAITRAKAEGFSAKMEPGISAEACLWADLGIDPGNSGHQSFEASQFMFFNHVPDPTTHLLLWQIAIAGEHTLTQFHTSSDRLQILVEQLNQWYPLDHEVVIYEAANLPIQAPRIERLPLANLPQAHLMPISTLLIPPAKKLEYNYAILAKLGIGPEDLG

SEQ ID NO: 2 - protein, 71 aa, Shewanella oneidensis
MSGLSDFFTQLGQDAQLMEDYKQNPEAVMRAHGLTDEQINAVMTGDMEKLKTLSGDSSYQSYLVISHGNGD

SEQ ID NO: 3 - protein, 262 aa, Streptomyces sp. NRRL S-118
MQETTGNAQLVVVGTGFRAIGDLTVEARACLEQADKVLCLIGDPLVTRHIEKLNASVETLDVHYAVGKPRSASYEDMVEHIMSELHRDQFVCVALYGHPGVFAYTGHEAIRRAREEGIAARMLPACSAEDWLFADLGLDPGERGCQSFEATDFLIRHRVFDPTGLLILWQVGVIGMIDRDPGYDARPGVTTLTDALVASYGSGHPVTVYEASPYVTAEPRTTTVPLAELPDTPLSAASTLVVPPLPPRPVDRELLARLAARR

SEQ ID NO: 4 - protein, 83 aa, Streptomyces sp. NRRL S-118
MPAAVVDFMEELVTQPRRQHAYRRSAEAYVADSALTASEREAVVSGDVDRMRAVLAEHSGVKEECHAVLVVIIFDPDEVPSGA

SEQ ID NO: 5 - protein, 409 aa, Rhodospirillum centenum SW
MRAAPMAETETPPAAPSPSAPERPRGSLTVVGTGLRALSHMTLEAISHIRDADRVFFSVPDGVTARQIRDINPEAVDLTQYYGEDKRRKQTYVQMSEVILREVRAGSAVTAVFYGHPGFFVFPARRILSIARKEGYRAVMLPGISSLDCLMADLRVDPSVNGCQILEATDLLLRNRPIITSGHVIILQVGSVGDSAFSFTAGFRHAKRAVLFERLIEAYGEEHRSVLYLAATYPGLDGQAVVRPLGAYRDPKVLASVPPAGTLYIPAKDMLPTDMAMAEKLGMSALVGPDAPVPAGPDSYGPFEAQAIAALDHYRPSPTWRPRTASKALQRVMTLLAGTPSVAAVYRKDPARLVDLHPDLTPAERKALLSRRAGPLNAVTAPPPEGAPPTVDEAGNGNGGDAPSEGETA

SEQ ID NO: 6 - protein, 125 aa, Rhodospirillum centenum SW
MTTIVPTELDQPDVIELSGGELDVAELSGGELDVAELFGGELDVAELSGGELDVAELSGGELDVAELSGGELDVAELSGGELDVAELSGGELDVAELSGGELDVAELSGGELDVAEIGIINTFDL

SEQ ID NO: 7 - protein, 442 aa, Burkholderia stabilis
MSEAKGRLVVIGSGIKAVSHFTLEAQAHIQQADIVLYAAADPVTDMWIESQNPNAFDLYQYYADDKARLITYVQMIERIMAEVRAGKYVCALFYGHPGVFVTPSHNAIAIARQEGFDAVMLPAVSAEDCLYADLGVDPSVPGMQIYEATDFLLRRRKVDTTANFVLWQVGCIGDLGFKFGGYKNDKFDVLLDYLEEIYGADHPAINYVANMFSGPPQIDRHVIGDYRDPEVKAKVSGISTFFIPAKDGIQSDAGMLAKLGLAKISEARRTPLICDREDYRVLADIARRNIHNHKVPPGYKYSFASDALYQTLLTLALDQRAQGEFQANPRAYLEARPGLTADERRSLLLQHTGVTRMMFKRDPDKEAVRFVGAALLDPALAREYRDRQAAAAQAVANHDIPVGEYEGVVASWLRGQGYAATPAAVTRAMEEVYTSKDHLSVA

SEQ ID NO: 8 - protein, 308 aa, Burkholderia stabilis
MASFDVSGTYMTSWLQPDGKTYIPGPVVIDTSNQSVTLNGATIASPSFTTTGVSWAATGGNATSAKLTFYQNGGVNGFVGLFSQGSDPLPAANNLFGNAGAATQQLSTWNGTYNLFSLTADGKSTPLSDKLVISAPQVTYGGSIVNKPVYTGEKGPKGEIDQLAWFSANGNPQNAIIQFSNSQAPTLTLYGELWNSGAQPTYVNLSGTTKSSSTPPTITVVAALISNTATEVTTEVTTEVTTEVTTEVTTEVTTEVTTEVLTEVAVAVVEVLAEDDDKPSPGSAKASLPKEDAAAAEKLLEKALALAE

SEQ ID NO: 9 - protein, 1034 aa, Achromobacter insuavis
MELAHQIYADIPSLSDTAAREPATATGELTIIGSGIGVMGFTRDAEQYIDDADHVVFCVADPATQVWLRGRRPDAIDLFALYDDRKPRYHTYMQMTEAMLYHVRRGKRVAAVFYGHPGIFVLSTHRAVAIARREGHRAQLRPGVSALDWLCADLGIDPAYPGMQTFEATDMLLRRRAIDPGSHVVLWQVGLIAEMGFRRRGFHNRRFDYLVEYLRGYYKPDHKIVHYIASRYPTLPPVIERYTLDELQNPEVHALITGISTFYVPPAQARAVDVDFAREVGLIQPGQHVGKPRLSRPLDRYGPREMRAIAALKDFRPPADYHFQTDTAAARFLLALSEQPELRRRFEANPEHALAAFPGLSAEERKQLSSRRTARTQRAARGAAVSISPGEQFVIDLETKPAVANQWMQLLNTAVKASPKNWQPVEDWLSQNYQGLALADVSDAVSSTQAWLLLMWNGVYATSGNPPQALFVMAGAPGDGAATSTVTSGTVYLNASPLFKISFSGGQLTFSQKDGNPCNGTLTFANPGASTPQSVSGMLWQDSQGKPSSNNFTATLQPLPSNPLSVWTGEYATVYTDNQQAGPGVAVFLPSTTNPSPTLYIGGQEVSGTSFANPTITWSGGQLSFALDASTTGALTLSGNIAGRAITGNTVASSATGFAGQYATQHVASVKGANVWQPFYPLGFTAPTSGSTTCTAWLGSQSFTAQFSNRQLTWSNGPSQIPNGQLSLSVNALTNAACFVGVTWANGDAKPTSPNIQGIASANSPANFVGNYNTTLDQQPAQVLSIGGDPNSIQVTYGSEKVTYSFFTGQLSGTTSSGLKLNAVFKYVTQGSHTPAQYVTAFTGTQTVNSAQHQWDSSQLSNDVQQWLGTYTTFLVDPGNGSLTAGGPTLTLSGTAASFTVQITADGTTTTLQNPKYQITGNTLYWSGENVSGGSNYNNGAISFYLDPRKQQRAFKGVFYANKPAPNAINWYGTPAGNNPSPPSPGFPWWGYLLIGLGIVGLGVGGLLLWRARASGYQRLATEDSIEMWEKKCQ

SEQ ID NO: 10 - protein, 111 aa, Achromobacter insuavis
MANTRLLEGGTCQDLEDDFDEQSEEFDNASDDASLNNPEELDIGDDVEIPDDPNVDVDVDADVEADIETEVDVDVDVDVTLEMFLEEDLERSEPGIDFVTGADGLDGDERP

SEQ ID NO: 11 - protein, 806 aa, Pseudomonas mosselii
MSMTTRNNREHAQATHYPSAEEHRRRWRRLAQAIAARAASISDDPALLVAPQRPGTLEILGSGIEASDFSRSDEARILVADHVFYCVADPATKIWILSQRPDAYDLYVLYDDSKPRYLTYMQMTEAMLHHVRNGEHVVAIFYGHPGVFVLSTHRAVTIARREGHHASMRAAVSALDTLCADLGVDPSQPGMQMYEATDMLIRRRQPDPGLHLVLWQVGLIGELGYRRQGYLNSNFAVLLDYLEDLYGPEHPVINYVGSRYPGIDPLIDRQTLASLRDPLAQSWVTGISTFYLPPRTAGQSDPQMLERLGLIRPGQPVRAASDPLRVIDRYDKRERRAFSDFARFDVPASYQWQADTGAGRFILALSDDAELRRQYRDDPQAAVQAWGGLDARERHLLGQRDPGAVQLAAKGADAQRHPGNREGVEHLLTYTSASSALHRALSAAAPGELRAAAAAWSRKAGLSIDWPGMNAELNALLQSSLAPWSGFYLDSSRRMSLSLFSRMKNAGLRVDLDGQPLVGVRYQSGVLTWSAEAGNGSSGYLQSDLSVQGGRSWIGLVWPAGEQAGSGYKVALRGQSLARPACLAVGDYRVAGEALRIVPSASASQGVDVLREGVALPGEVLFNGRDTRIGDRRLALSTRRFEDLAQWAQGAYRLRLVHGRLAELLALRLGPYGLEIAGKPVTAEFREGRIQWQGGPPAVESGQLDVTLDPITLKPLLHGQGRSAAGNKVQLRGMALIEPLWIDQLLEQPRLGLPEWAWRHLVAVMVAASDKGGIFLWHGYDRARGNLRLLREALARLRQDEMEDAG

SEQ ID NO: 12 - protein, 605 aa, Pseudomonas mosselii
MATYPVQITNNTNLALDIYTTLNKNPATDPPSTNPADYTAVYTLQGSVGANQSTTLPLTESLARLVIVRQSDQFPLLVQVANALLPDSEQVQVGNQDVTTANSGWAFYQSFISQPFTPTALEFSELVSETPANALNDKAASFFAANGYPGVSFALFSALGYWANNQLYAYPGTYYCYEPPSGNSMGFILPTTSVGTLTIAGGKANYSPSTGGSTALQFQYGQLTSPGADDKHGFNTTGFIRDLTWEGKPDVITWAFVGTYDGQQFIAQSYQSPQLPWYAVAYDMAYGALFTVQLAMTLDAAINLLGTVANGMQWLAQNTGKLISRIQDSLNSTGDTAGAGSGVGDAADPVNVDVDIDVDIDVDVDVDIDIDIDVDVDVDIDIDVDVDFIAVVDVDVDVDIDVDIDVVTDTETDIDIDVDVDIDTDVNVEPGALMKVVNGVGNWIMTKALPTLIEGAVIYVAFQSVGAIFQAWKNQDEKDIENLQPRQSTGVGVLVNYMLQDDKPVAARWQTFSQYVAEVQGDPKTVGVTISTLLQTGNTKADNDAANWRWSSDDENQVVASMAPYTGDQACKAFVILGNATYQGKPLPVKVGASVAMKYLAAQGA

SEQ ID NO: 13 - protein, 372 aa, Brevibacillus laterosporus PE36
MKKGSLIVVGSGIKGVAHFTVEAQGWIREADVVPYCVSDPVSEVWIKENSKHSIDLYQFYGNEKQRINTYNEMVDEILSHVRSGKDTCAVFYGHPGIFVHPSHKAISIARSEGYKAAMLPGISALDCLCADLGVDPSVTGMQTVEATDLLLRNRRLNTDQNVVIWQIGCVGDLGFNFSGYDNRNLKILVEYLEKFYAKDHMVTHYQGSQYSICPPSIAKMPLSELKNAPVTGISTLYIPPQDKLKLDEEMVQRLGLRKTQTVNTQKVAEPARKTDQQKVQPRKASYNQYVAAPDHSELANFLAELSESPLLLAQFMRNPEITSSLMADLSPSEKDALLSQHPGKIRMAIKLSSWNKHLKGVSTLIKQRENNF

SEQ ID NO: 14 - protein, 590 aa, Brevibacillus laterosporus PE36
MNKRTENNIESYDDAQKFFQQLLINPQLAKEYVEPIQQAQINENPDVITTWLASKGYNTNPDEITQAQEQMQNTDLIYWAGIYGRTSVSSDIPPNKNSVFKPGPPLVVQDDKTVVLNSIPLKNFTFKNKTLTWGFSDNSTAGSITFVEVPSVNSDSTAPQSYTGKEFTGTIQMSSGEEKEFYSGQIGPITAFPLANWSGYYGETSIEQSANNYIAGPTLIVRDNETVLLNNQPLVNFTYDNIKNQLTWSQQDNDSSGSIYFARTTTPTQTGYVGLYFHGTIKQSSDQSLPYTGHISTPKKLEDWTGVYGQTVLTDSDNKHSQGPELKVISNTRVTLDGVDLKNFSYDENQKLLTWSIADNSTAGNITFGKITTPTSQGYVGNYFEGTLQQSKASEPLKYFGEVGTASSYSGGYQPSTVEKVFQILGYVATVAGLAQMVYVGYKVGKWAWEKLFKSQSETSEITDGLAEGVEPELVRYTPIDPENPPPINTTSETTTETVAEPVTTTETEVVTSEVTSEVTTEVTTEVTTEVTTEVVEVVEVVEVVEVVEVVEVVEVVEVVEVVEVVEVVFAEDEEKDEEKDEDEDVKKED

SEQ ID NO: 15 - protein, 535 aa, Spirosoma linguale DSM74
MNKNGKLIVIGSGIKSIAHFTLESQAHLQQADIVLYAASDPVTDMWIQKQNPNSFDLYQYYGNTKNRIITYTQMIERVMMELRSGKYVCALFYGHPGVFVTPSHNAIELARREGYEAEMLPGISAEDCLFADLGVDPSIPGLQTYEATDLLLRQRSINTEINAVIWQVGCVGDVGFKFHGYDNEKLNILLDYLDKFYPPDQIVYNYVASMFSMAKPKKDKFKLSDFRDPSIAKEVTGISTFFIPAVTMTESDIEMSKKLGLKSGKSRSNPLICDHELYPSYKKVALKNISEHLIPEGYKFSHSSDALYNLVTRLALDFKELYKYRENPSAYLKLIPELTPTERNMLTMQHHGALRMLFKRDRMEEARLFVDEAIKKPTIANEYLKKQQAEYDALINEEISESDYEARLVKWFLDKGYATTPSAVTASINVLEMPDIIDFSGEYQCVLTMNDSKQKVSVKVNILDQTIAVNNELIETPYFGGDHVIWSKEDKNNSTGVLTFSKQLNAFILEGKYANNSSVLPETYNLHGVTDTAYN

SEQ ID NO: 16 - protein, 299 aa, Spirosoma linguale DSM74
MTATNFSGNYMSFVLGVDGNTWGPGPVVAVNTTSQTLFIDGVQVNNPTFTSTSVMWMASSNNPSSGSIKFYSSAPDTGGFIGTYVEGTAPLPSSNNFSGVSSAAPDDLSVWNGTYNTFILNGSKWTQDSTLIVAAPNISYNGKDISNYIYMGTKSTQNLDQLSWFVAGGNAQNAIVEFFKDSSGYLTFGGTQWVSGDAPAANNFIGTTKATPEPPTITAVVAIVINSSTTEVAEVTEVTEVTEVTEVTEVVEVVEVVEVVEVIAAAEEAEVKGQQQQSKISTAQQDQEAAYKLSRKSNG

SEQ ID NO: 17 - protein, 78 aa, Artificial Sequence
  Synthetic - wild-type SonA with N-terminal His-tag
MHHHHHHMSGLSDFFTQLGQDAQLMEDYKQNPEAVMRAHGLTDEQINAVMTGDMEKLKTLSGDSSYQSYLVISHGNGD

SEQ ID NO: 18 - protein, 82 aa, Artificial Sequence
  Synthetic - Extcore SonA mutant with N-terminal His-tag
MHHHHHHMSGLSDFFTQLGQDAQLMEDYKQNPEAVMRAHGLTDEQINAVMTGDMEKLKTLSGDSSYQSYLVIVLVISHGNGD

SEQ ID NO: 19 - protein, 81 aa, Artificial Sequence
  Synthetic - KTLext SonA mutant with N-terminal His-tag
MHHHHHHMSGLSDFFTQLGQDAQLMEDYKQNPEAVMRAHGLTDEQINAVMTGDMEKLKTLKTLSGDSSYQSYLVISHGNGD

SEQ ID NO: 20 - protein, 75 aa, Artificial Sequence
  Synthetic - KTLdel SonA mutant with N-terminal His-tag
MHHHHHHMSGLSDFFTQLGQDAQLMEDYKQNPEAVMRAHGLTDEQINAVMTGDMEKLSGDSSYQSYLVISHGNGD

SEQ ID NO: 21 - protein, 78 aa, Artificial Sequence
  Synthetic - L63G SonA mutant with N-terminal His-tag
MHHHHHHMSGLSDFFTQLGQDAQLMEDYKQNPEAVMRAHGLTDEQINAVMTGDMEKLKTLSGDSSYQSYGVISHGNGD

SEQ ID NO: 22 - protein, 78 aa, Artificial Sequence
  Synthetic
  MISC_FEATURE (1)..(78): I65G SonA mutant with N-terminal His-tag
MHHHHHHMSGLSDFFTQLGQDAQLMEDYKQNPEAVMRAHGLTDEQINAVMTGDMEKLKTLSGDSSYQSYLVGSHGNGD

SEQ ID NO: 23 - protein, 78 aa, Artificial Sequence
  Synthetic
  MISC_FEATURE (1)..(78): L63W SonA mutant with N-terminal His-tag
MHHHHHHMSGLSDFFTQLGQDAQLMEDYKQNPEAVMRAHGLTDEQINAVMTGDMEKLKTLSGDSSYQSYWVISHGNGD

SEQ ID NO: 24 - protein, 78 aa, Artificial Sequence
  Synthetic
  MISC_FEATURE (1)..(78): I65W SonA mutant with N-terminal His-tag
MHHHHHHMSGLSDFFTQLGQDAQLMEDYKQNPEAVMRAHGLTDEQINAVMTGDMEKLKTLSGDSSYQSYLVWSHGNGD

SEQ ID NO: 25 - protein, 78 aa, Artificial Sequence
  Synthetic
  MISC_FEATURE (1)..(78): L63Q SonA mutant with N-terminal His-tag
MHHHHHHMSGLSDFFTQLGQDAQLMEDYKQNPEAVMRAHGLTDEQINAVMTGDMEKLKTLSGDSSYQSYQVISHGNGD

SEQ ID NO: 26 - protein, 78 aa, Artificial Sequence
  Synthetic
  MISC_FEATURE (1)..(78): I65Q SonA mutant with N-terminal His-tag
MHHHHHHMSGLSDFFTQLGQDAQLMEDYKQNPEAVMRAHGLTDEQINAVMTGDMEKLKTLSGDSSYQSYLVQSHGNGD

SEQ ID NO: 27 - protein, 78 aa, Artificial Sequence
  Synthetic
  MISC_FEATURE (1)..(78): L63M SonA mutant with N-terminal His-tag
MHHHHHHMSGLSDFFTQLGQDAQLMEDYKQNPEAVMRAHGLTDEQINAVMTGDMEKLKTLSGDSSYQSYMVISHGNGD

SEQ ID NO: 28 - protein, 78 aa, Artificial Sequence
  Synthetic
  MISC_FEATURE (1)..(78): I65M SonA mutant with N-terminal His-tag
MHHHHHHMSGLSDFFTQLGQDAQLMEDYKQNPEAVMRAHGLTDEQINAVMTGDMEKLKTLSGDSSYQSYLVMSHGNGD

SEQ ID NO: 29 - protein, 78 aa, Artificial Sequence
  Synthetic
  MISC_FEATURE (1)..(78): L63H SonA mutant with N-terminal His-tag
MHHHHHHMSGLSDFFTQLGQDAQLMEDYKQNPEAVMRAHGLTDEQINAVMTGDMEKLKTLSGDSSYQSYHVISHGNGD

SEQ ID NO: 30 - protein, 78 aa, Artificial Sequence
  Synthetic
  MISC_FEATURE (1)..(78): I65H SonA mutant with N-terminal His-tag
MHHHHHHMSGLSDFFTQLGQDAQLMEDYKQNPEAVMRAHGLTDEQINAVMTGDMEKLKTLSGDSSYQSYLVHSHGNGD

SEQ ID NO: 31 - protein, 78 aa, Artificial Sequence
  Synthetic
  MISC_FEATURE (1)..(78): L63K SonA mutant with N-terminal His-tag
MHHHHHHMSGLSDFFTQLGQDAQLMEDYKQNPEAVMRAHGLTDEQINAVMTGDMEKLKTLSGDSSYQSYKVISHGNGD

SEQ ID NO: 32 - protein, 78 aa, Artificial Sequence
  Synthetic
  MISC_FEATURE (1)..(78): I65K SonA mutant with N-terminal His-tag
MHHHHHHMSGLSDFFTQLGQDAQLMEDYKQNPEAVMRAHGLTDEQINAVMTGDMEKLKTLSGDSSYQSYLVKSHGNGD

SEQ ID NO: 33 - protein, 78 aa, Artificial Sequence
  Synthetic
  MISC_FEATURE (1)..(78): L63D SonA mutant with N-terminal His-tag
MHHHHHHMSGLSDFFTQLGQDAQLMEDYKQNPEAVMRAHGLTDEQINAVMTGDMEKLKTLSGDSSYQSYDVISHGNGD

SEQ ID NO: 34 - protein, 78 aa, Artificial Sequence
  Synthetic
  MISC_FEATURE (1)..(78): I65D SonA mutant with N-terminal His-tag
MHHHHHHMSGLSDFFTQLGQDAQLMEDYKQNPEAVMRAHGLTDEQINAVMTGDMEKLKTLSGDSSYQSYLVDSHGNGD

SEQ ID NO: 35 - protein, 78 aa, Artificial Sequence
  Synthetic
  MISC_FEATURE (1)..(78): L63P SonA mutant with N-terminal His-tag
MHHHHHHMSGLSDFFTQLGQDAQLMEDYKQNPEAVMRAHGLTDEQINAVMTGDMEKLKTLSGDSSYQSYPVISHGNGD

SEQ ID NO: 36 - protein, 78 aa, Artificial Sequence
  Synthetic
  MISC_FEATURE (1)..(78): I65P SonA mutant with N-terminal His-tag
MHHHHHHMSGLSDFFTQLGQDAQLMEDYKQNPEAVMRAHGLTDEQINAVMTGDMEKLKTLSGDSSYQSYLVPSHGNGD

SEQ ID NO: 37 - protein, 74 aa, Artificial Sequence
  Synthetic
  MISC_FEATURE (1)..(74): KTLSdel SonA mutant with N-terminal His-tag
MHHHHHHMSGLSDFFTQLGQDAQLMEDYKQNPEAVMRAHGLTDEQINAVMTGDMEKLGDSSYQSYLVISHGNGD

SEQ ID NO: 38 - protein, 82 aa, Artificial Sequence
  Synthetic
  MISC_FEATURE (1)..(82): KTLSext SonA mutant with N-terminal His-tag
MHHHHHHMSGLSDFFTQLGQDAQLMEDYKQNPEAVMRAHGLTDEQINAVMTGDMEKLKTLSKTLSGDSSYQSYLVISHGNGD

SEQ ID NO: 39 - protein, 71 aa, Artificial Sequence
  Synthetic
  MISC_FEATURE (1)..(71): KTLSGDSdel SonA mutant with N-terminal His-tag
MHHHHHHMSGLSDFFTQLGQDAQLMEDYKQNPEAVMRAHGLTDEQINAVMTGDMEKLSYQSYLVISHGNGD

SEQ ID NO: 40 - protein, 71 aa, Artificial Sequence
  Synthetic
  MISC_FEATURE (1)..(71): MEKLKTLdel SonA mutant with N-terminal His-tag
MHHHHHHMSGLSDFFTQLGQDAQLMEDYKQNPEAVMRAHGLTDEQINAVMTGDSGDSSYQSYLVISHGNGD

SEQ ID NO: 41 - protein, 85 aa, Artificial Sequence
  Synthetic
  MISC_FEATURE (1)..(85): MEKLKTLext SonA mutant with N-terminal His-tag
MHHHHHHMSGLSDFFTQLGQDAQLMEDYKQNPEAVMRAHGLTDEQINAVMTGDMEKLKTLMEKLKTLSGDSSYQSYLVISHGNGD

SEQ ID NO: 42 - protein, 75 aa, Artificial Sequence
  Synthetic
  MISC_FEATURE (1)..(75): QSYdel SonA mutant with N-terminal His-tag
MHHHHHHMSGLSDFFTQLGQDAQLMEDYKQNPEAVMRAHGLTDEQINAVMTGDMEKLKTLSGDSSYLVISHGNGD

SEQ ID NO: 43 - DNA, 40 bp, Artificial Sequence
  Synthetic
  misc_feature (1)..(40): prmMRJ036_fw primer
actttaagaaggagatataccatgggatcactcgtctgtg

SEQ ID NO: 44 - DNA, 53 bp, Artificial Sequence
  Synthetic
  misc_feature (1)..(53): pRMMRJ045_rev primer
gatgatgatgatgatgcatgttttctccttattgttaataatgattcaataac

SEQ ID NO: 45 - DNA, 57 bp, Artificial Sequence
  Synthetic
  misc_feature (1)..(57): prmMRJ044_fw primer
aggagaaaacatgcatcatcatcatcatcacatgtctggattatcggatttttttac

SEQ ID NO: 46 - DNA, 45 bp, Artificial Sequence
  Synthetic
  misc_feature (1)..(45): pRMMRJ045_rev primer
cgagtgcggccgcaagcttgtcgacttaatcaccattaccatgtg

SEQ ID NO: 47 - DNA, 20 bp, Artificial Sequence
  Synthetic
  misc_feature (1)..(20): T7_fw primer
taatacgactcactataggg

SEQ ID NO: 48 - DNA, 19 bp, Artificial Sequence
  Synthetic
  misc_feature (1)..(19): T7_rv primer
gctagttattgctcagcgg

SEQ ID NO: 49 - DNA, 36 bp, Artificial Sequence
  Synthetic
  misc_feature (1)..(36): prFM1175 primer
tttaagaaggagatatacatgcatcatcatcatcat

SEQ ID NO: 50 - DNA, 36 bp, Artificial Sequence
  Synthetic
  misc_feature (1)..(36): prFM1176 primer
agtgcggccgcaagcttgttaatcaccattaccatg

SEQ ID NO: 51 - DNA, 58 bp, Artificial Sequence
  Synthetic
  misc_feature (1)..(58): prFM1177 primer
taagaaggagatatacatgcatcatcatcatcatcacagcagcatgggatcactcgtc

SEQ ID NO: 52 - DNA, 36 bp, Artificial Sequence
  Synthetic
  misc_feature (1)..(36): prFM1178 primer
agtgcggccgcaagcttgttatcccaaatcttcggg

SEQ ID NO: 53 - DNA, 20 bp, Artificial Sequence
  Synthetic
  misc_feature (1)..(20): prmMRJ068 primer
atataacatatgccggcggc

SEQ ID NO: 54 - DNA, 27 bp, Artificial Sequence
  Synthetic
  misc_feature (1)..(27): prmMRJ069 primer
ttatatggatccttacgcaccgctcgg

SEQ ID NO: 55 - DNA, 25 bp, Artificial Sequence
  Synthetic
  misc_feature (1)..(25): prmMRJ066 primer
atataacatatgcaggagaccaccg

SEQ ID NO: 56 - DNA, 26 bp, Artificial Sequence
  Synthetic
  misc_feature (1)..(26): prmMRJ067 primer
ttatatggatccttaacgacgcgccg

SEQ ID NO: 57 - DNA, 26 bp, Artificial Sequence
  Synthetic
  misc_feature (1)..(26): prmMRJ064 primer
atataacatatgacgaccatcgtccc

SEQ ID NO: 58 - DNA, 27 bp, Artificial Sequence
  Synthetic
  misc_feature (1)..(27): prmMRJ063 primer
ttatatggatcctcaggcggtttcccc

SEQ ID NO: 59 - DNA, 24 bp, Artificial Sequence
  Synthetic
  misc_feature (1)..(24): prmMRJ062 primer
atataacatatgagagccgccccg

SEQ ID NO: 60 - DNA, 27 bp, Artificial Sequence
  Synthetic
  misc_feature (1)..(27): prmMRJ063primer
ttatatggatcctcaggcggtttcccc

SEQ ID NO: 61 - DNA, 38 bp, Artificial Sequence
  Synthetic
  misc_feature (1)..(38): F_BstA_pET28HisSumo primer
ccggtggcgcttccttcgatgtgtccggaacatacatg

SEQ ID NO: 62 - DNA, 29 bp, Artificial Sequence
  Synthetic
  misc_feature (1)..(29): R_BstA_pET28HisSumo primer
ccgcaagctttcattccgccagcgccagc

SEQ ID NO: 63 - DNA, 27 bp, Artificial Sequence
  Synthetic
  misc_feature (1)..(27): F_BstMT_pET28HisSumo primer
ccggtggcagcgaggccaaaggcaggc

SEQ ID NO: 64 - DNA, 31 bp, Artificial Sequence
  Synthetic
  misc_feature (1)..(31): R_BstMT_pET28HisSumo primer
gccgcaagctttcaggccacgctcaggtggt

SEQ ID NO: 65 - DNA, 41 bp, Artificial Sequence
  Synthetic
  misc_feature (1)..(41): F_BstA_coexp_HindIII primer
gcggaagcttaatacgactcactataggggaattgtgagcg

SEQ ID NO: 66 - DNA, 29 bp, Artificial Sequence
  Synthetic
  misc_feature (1)..(29): R_BstA_coexp_XhoI primer
aatcctcgagtcaggccacgctcaggtgg

SEQ ID NO: 67 - DNA, 54 bp, Artificial Sequence
  Synthetic
  misc_feature (1)..(54): Fwd_pET28b_NHis_Pmosselii_Repeat_RiPP primer
gctcgagtgcggccgcaagcttgcatgttcatccggcatcctccatttcatcct

SEQ ID NO: 68 - DNA, 54 bp, Artificial Sequence; identical to SEQ ID NO: 67
  Synthetic
  misc_feature (1)..(54): R_Pmosselii_NHisRepRiPP_HindIII primer

SEQ ID NO: 69 - DNA, 23 bp, Artificial Sequence
  Synthetic
  misc_feature (1)..(23): Rev_pET28b_NdeI_Backbone_Gibson primer
catatggctgccgcgcggcacca

SEQ ID NO: 70 - DNA, 28 bp, Artificial Sequence
  Synthetic
  misc_feature (1)..(28): F_backbone_pET28NHisSumoPmoA primer
gcgcctgaaagcttgcggccgcactcga

SEQ ID NO: 71 - protein, 12 aa, Artificial Sequence
  Synthetic
  MISC_FEATURE (1)..(12): Fragment of SEQ ID NO2 shown in FIG. 3
QSYLVISHGNGD

SEQ ID NO: 72 - protein, 16 aa, Artificial Sequence
  Synthetic
  MISC_FEATURE (1)..(16): Fragment of SEQ ID NO2 shown in FIG. 5 and FIG. 7
DSSYQSYLVISHGNGD

SEQ ID NO: 73 - protein, 10 aa, Artificial Sequence
  Synthetic
  MISC_FEATURE (1)..(10): Fragment of SEQ ID NO4 shown in FIG. 9 and FIG. 10
CHAVLVVIIF

SEQ ID NO: 74 - protein, 10 aa, Artificial Sequence
  Synthetic
  MISC_FEATURE (1)..(10): Fragment of SEQ ID NO6 shown in FIG. 11 and FIG. 12
DVAELSGGEL

SEQ ID NO: 75 - protein, 10 aa, Artificial Sequence
  Synthetic
  MISC_FEATURE (1)..(10): Fragment of SEQ ID NO6 shown in FIG. 12
DVIELSGGEL

SEQ ID NO: 76 - protein, 10 aa, Artificial Sequence
  Synthetic
  MISC_FEATURE (1)..(10): Fragment of SEQ ID NO6 shown in FIG. 12
DVAELFGGEL

SEQ ID NO: 77 - protein, 11 aa, Artificial Sequence
  Synthetic
  MISC_FEATURE (1)..(11): Fragment of SEQ ID NO6 shown in FIG. 12
DVAEIGIINTF

SEQ ID NO: 78 - protein, 66 aa, Artificial Sequence
  Synthetic
  MISC_FEATURE (1)..(66): Fragment of SEQ ID NO8 shown in FIG. 13
TPPTITVVAALISNTATEVTTEVTTEVTTEVTTEVTTEVTTEVTTEVLTEVAVAVVEVLAEDDDKP

SEQ ID NO: 79 - protein, 23 aa, Artificial Sequence
  Synthetic
  MISC_FEATURE (1)..(23): Fragment of SEQ ID NO8 shown in FIG. 14
SSSTPPTITVVAALISNTATEVT

SEQ ID NO: 80 - protein, 19 aa, Artificial Sequence
  Synthetic
  MISC_FEATURE (1)..(19): Fragment of SEQ ID NO8 shown in FIG. 14
SNTATEVTTEVTTEVTTEV

SEQ ID NO: 81 - protein, 24 aa, Artificial Sequence
  Synthetic
  MISC_FEATURE (1)..(24): Fragment of SEQ ID NO8 shown in FIG. 14
TEVTTEVTTEVTTEVTTEVLTEVA

SEQ ID NO: 82 - protein, 13 aa, Artificial Sequence
  Synthetic
  MISC_FEATURE (1)..(13): Fragment of SEQ ID NO8 shown in FIG. 14
TEVAVAVVEVLAE

SEQ ID NO: 83 - protein, 52 aa, Artificial Sequence
  Synthetic
  MISC_FEATURE (1)..(52): Fragment of SEQ ID NO10 shown in FIG. 15
PDDPNVDVDVDADVEADIETEVDVDVDVDVTLEMFLEEDLERSEPGIDFVTG

SEQ ID NO: 84 - protein, 20 aa, Artificial Sequence
  Synthetic
  MISC_FEATURE (1)..(20): Fragment of SEQ ID NO10 shown in FIG. 16
VEIPDDPNVDVDVDADVEAD

SEQ ID NO: 85 - protein, 18 aa, Artificial Sequence
  Synthetic
  MISC_FEATURE (1)..(18): Fragment of SEQ ID NO10 shown in FIG. 16
EIPDDPNVDVDVDADVEA

SEQ ID NO: 86 - protein, 15 aa, Artificial Sequence
  Synthetic
  MISC_FEATURE (1)..(15): Fragment of SEQ ID NO10 shown in FIG. 16
TEVDVDVDVDVTLEM

SEQ ID NO: 87 - protein, 9 aa, Artificial Sequence
  Synthetic
  MISC_FEATURE (1)..(9): Fragment of SEQ ID NO10 shown in FIG. 16
EPGIDFVTG

SEQ ID NO: 88 - protein, 65 aa, Artificial Sequence
  Synthetic
  MISC_FEATURE (1)..(65): Fragment of SEQ ID NO12 shown in FIG. 17
DAADPVNVDVDIDVDIDVDVDVDIDIDIDVDVDVDIDIDVDVDFIAVVDVDVDVDIDVDIDVVTD

SEQ ID NO: 89 - protein, 19 aa, Artificial Sequence
  Synthetic
  MISC_FEATURE (1)..(19): Fragment of SEQ ID NO12 shown in FIG. 18
ADPVNVDVDIDVDIDVDVD

SEQ ID NO: 90 - protein, 15 aa, Artificial Sequence
  Synthetic
  MISC_FEATURE (1)..(15): Fragment of SEQ ID NO12 shown in FIG. 18
IAVVDVDVDVDIDVD

SEQ ID NO: 91 - protein, 17 aa, Artificial Sequence
  Synthetic
  MISC_FEATURE (1)..(17): Fragment of SEQ ID NO12 shown in FIG. 18
IAVVDVDVDVDIDVDID

SEQ ID NO: 92 - protein, 34 aa, Artificial Sequence
  Synthetic
  MISC_FEATURE (1)..(34): Fragment of SEQ ID NO14 shown in FIG. 19
PINTTSETTTETVAEPVTTTETEVVTSEVTSEVT

SEQ ID NO: 93 - protein, 16 aa, Artificial Sequence
  Synthetic
  MISC_FEATURE (1)..(16): Fragment of SEQ ID NO14 shown in FIG. 19
TVAEPVTTTETEVVTS

SEQ ID NO: 94 - protein, 23 aa, Artificial Sequence
  Synthetic
  MISC_FEATURE (1)..(23): Fragment of SEQ ID NO14 shown in FIG. 20
TVAEPVTTTETEVVTSEVTSEVT

SEQ ID NO: 95 - protein, 21 aa, Artificial Sequence
  Synthetic
  MISC_FEATURE (1)..(21): Fragment of SEQ ID NO14 shown in FIG. 20
TVAEPVTTTETEVVTSEVTSE

SEQ ID NO: 96 - protein, 38 aa, Artificial Sequence
  Synthetic
  MISC_FEATURE (1)..(38): Fragment of SEQ ID NO16 shown in FIG. 21
PPTITAVVAIVINSSTTEVAEVTEVTEVTEVTEVTEVV

SEQ ID NO: 97 - protein, 15 aa, Artificial Sequence
  Synthetic
  MISC_FEATURE (1)..(15): Fragment of SEQ ID NO16 shown in FIG. 22
EVAEVTEVTEVTEVT

SEQ ID NO: 98 - protein, 18 aa, Artificial Sequence
  Synthetic
  MISC_FEATURE (1)..(18): Fragment of SEQ ID NO16 shown in FIG. 22
TAVVAIVINSSTTEVAEV

SEQ ID NO: 99 - protein, 24 aa, Artificial Sequence
  Synthetic
  MISC_FEATURE (1)..(24): Fragment of SEQ ID NO16 shown in FIG. 22
ATPEPPTITAVVAIVINSSTTEVA

SEQ ID NO: 100 - protein, 20 aa, Artificial Sequence
  Synthetic
  MISC_FEATURE (1)..(20): Fragment of SEQ ID NO18 shown in FIG. 7D
DSSYQSYLVIVLVISHGNGD

SEQ ID NO: 101 - protein, 16 aa, Artificial Sequence
  Synthetic
  MISC_FEATURE (1)..(16): Fragment of SEQ ID NO19 shown in FIG. 7E
DSSYQSYLVISHGNGD

SEQ ID NO: 102 - protein, 16 aa, Artificial Sequence
  Synthetic
  MISC_FEATURE (1)..(16): Fragment of SEQ ID NO19 shown in FIG. 7E
DSSYQSYLVISHGNGD

SEQ ID NO: 103 - protein, 16 aa, Artificial Sequence
  Synthetic
  MISC_FEATURE (1)..(16): Fragment of SEQ ID NO21 shown in FIG. 7G
DSSYQSYGVISHGNGD

SEQ ID NO: 104 - protein, 16 aa, Artificial Sequence
  Synthetic
  MISC_FEATURE (1)..(16): Fragment of SEQ ID NO22 shown in FIG. 7H
DSSYQSYLVGSHGNGD

SEQ ID NO: 105 - protein, 16 aa, Artificial Sequence
  Synthetic
  MISC_FEATURE (1)..(16): Fragment of SEQ ID NO23 shown in FIG. 7I
DSSYQSYWVISHGNGD

SEQ ID NO: 106 - protein, 16 aa, Artificial Sequence
  Synthetic
  MISC_FEATURE (1)..(16): Fragment of SEQ ID NO24 shown in FIG. 7J
DSSYQSYLVWSHGNGD

SEQ ID NO: 107 - protein, 16 aa, Artificial Sequence
  Synthetic
  MISC_FEATURE (1)..(16): Fragment of SEQ ID NO25 shown in FIG. 7K
DSSYQSYQVISHGNGD

SEQ ID NO: 108 - protein, 16 aa, Artificial Sequence
  Synthetic
  MISC_FEATURE (1)..(16): Fragment of SEQ ID NO26 shown in FIG. 7L
DSSYQSYLVQSHGNGD

SEQ ID NO: 109 - protein, 16 aa, Artificial Sequence
  Synthetic
  MISC_FEATURE (1)..(16): Fragment of SEQ ID NO27 shown in FIG. 7M
DSSYQSYMVISHGNGD

SEQ ID NO: 110 - protein, 16 aa, Artificial Sequence
  Synthetic
  MISC_FEATURE (1)..(16): Fragment of SEQ ID NO28 shown in FIG. 7N
DSSYQSYLVMSHGNGD

SEQ ID NO: 111 - protein, 16 aa, Artificial Sequence
  Synthetic
  MISC_FEATURE (1)..(16): Fragment of SEQ ID NO29 shown in FIG. 7O
DSSYQSYHVISHGNGD

SEQ ID NO: 112 - protein, 16 aa, Artificial Sequence
  Synthetic
  MISC_FEATURE (1)..(16): Fragment of SEQ ID NO30 shown in FIG. 7P
DSSYQSYLVHSHGNGD

SEQ ID NO: 113 - protein, 16 aa, Artificial Sequence
  Synthetic
  MISC_FEATURE (1)..(16): Fragment of SEQ ID NO31 shown in FIG. 7Q
DSSYQSYKVISHGNGD

SEQ ID NO: 114 - protein, 16 aa, Artificial Sequence
  Synthetic
  MISC_FEATURE (1)..(16): Fragment of SEQ ID NO32 shown in FIG. 7R
DSSYQSYLVKSHGNGD

SEQ ID NO: 115 - protein, 20 aa, Artificial Sequence
  Synthetic
  MISC_FEATURE (1)..(20): Fragment of SEQ ID NO33 shown in FIG. 7S
TLSGDSSYQSYDVISHGNGD

SEQ ID NO: 116 - protein, 20 aa, Artificial Sequence
  Synthetic
  MISC_FEATURE (1)..(20): Fragment of SEQ ID NO34 shown in FIG. 7T
TLSGDSSYQSYLVDSHGNGD

SEQ ID NO: 117 - protein, 16 aa, Artificial Sequence
  Synthetic
  MISC_FEATURE (1)..(16): Fragment of SEQ ID NO35 shown in FIG. 7U
DSSYQSYPVISHGNGD

SEQ ID NO: 118 - protein, 16 aa, Artificial Sequence
  Synthetic
  MISC_FEATURE (1)..(16): Fragment of SEQ ID NO36 shown in FIG. 7V
DSSYQSYLVPSHGNGD

SEQ ID NO: 119 - protein, 16 aa, Artificial Sequence
  Synthetic
  MISC_FEATURE (1)..(16): Fragment of SEQ ID NO37 shown in FIG. 7W
DSSYQSYLVISHGNGD

SEQ ID NO: 120 - protein, 20 aa, Artificial Sequence
  Synthetic
  MISC_FEATURE (1)..(20): Fragment of SEQ ID NO38 shown in FIG. 7X
TLSGDSSYQSYLVISHGNGD

SEQ ID NO: 121 - protein, 15 aa, Artificial Sequence
  Synthetic
  MISC_FEATURE (1)..(15): Fragment of SEQ ID NO39 shown in FIG. 7Y
LSYQSYLVISHGNGD

SEQ ID NO: 122 - protein, 16 aa, Artificial Sequence
  Synthetic
  MISC_FEATURE (1)..(16): Fragment of SEQ ID NO40 shown in FIG. 7Z
DSSYQSYLVISHGNGD

SEQ ID NO: 123 - protein, 20 aa, Artificial Sequence
  Synthetic
  MISC_FEATURE (1)..(20): Fragment of SEQ ID NO41 shown in FIG. 7AA
TLSGDSSYQSYLVISHGNGD

SEQ ID NO: 124 - protein, 13 aa, Artificial Sequence
  Synthetic
  MISC_FEATURE (1)..(13): Fragment of SEQ ID NO42 shown in FIG. 7BB
DSSYLVISHGNGD

SEQ ID NO: 125 - protein, 243 aa, Artificial Sequence
  Synthetic
  MISC_FEATURE (1)..(243): Bacterial alpha-N-methyltransferase domain shown in FIG. 2A
GKLVIVGSGIGSIGQFTLSAVAHIEQADRVFFVVADPATEAFIYSKNKNSVDLYKFYDDKKPRMDTYIQMAEVMLRELRKGYSVVGVIYGHPGVFVTPSHRAISIARDEGYSAKMLPGVSAEDNLFADIGIDPSRPGCLTYEATDLLLRNRTLVPSSHLVLFQVGCIGLSDFRFKGFDNINFDVLLDRLEQVYGPDHAVIHYMAAVLPQSTTTIDRYTIKELRDPVIKKRITAISTFYLPPKA

SEQ ID NO: 126 - protein, 241 aa, Artificial Sequence
  Synthetic
  MISC_FEATURE (1)..(241): Bacterial alpha-N-methyltransferase domain shown in FIG. 2A
GTLTIAGSGIASIGHITLETLSYIQEADKVYYAITDPATEAFIQDKSEGDCFDLTVYYDKNKIRYETYVQMCEVMLRDVRADYNVVGVFYGHPGVFVSPSHRAIAIARDEGYRARMLPGVSAEDYMFSDLGFDPAVPGCMTQEATAMLNHNKKLDPSIHNIIWQVGAVGIDTMVFDNRKFHLLVDRLEEDFGPDHRVVNYIGAVLPQSTTVMDEFTIGDLRKEDVVKQFTTVSTFYVPPRT

SEQ ID NO: 127 - protein, 242 aa, Artificial Sequence
  Synthetic
  MISC_FEATURE (1)..(242)
  MISC_FEATURE (1)..(242): Bacterial alpha-N-methyltransferase domain shown in FIG. 2A
GKLILVGTGVRSLCQLTLEAIDEIERADVIYYAVRDATTEGFIKKRNKEAIDLYQYFINDEEIPEADIYIQIAEVMLAATRKGRRVVGAFFGHPGLFMSPNRRALAIAQAEGYTAKILPGVSVDDCLLADLGVDPSFIGCLTCEARDFMIHDHLGLTSRHVIMYEVGYLGFYGDDSKTDYFEYFVNRLEEIYGNEHSLVNYTAAISPLMQPVINTLTIGDLRKPEVRKQITSASTLYFPPKE

SEQ ID NO: 128 - protein, 243 aa, Artificial Sequence
  Synthetic
  MISC_FEATURE (1)..(243): Bacterial alpha-N-methyltransferase domain shown in FIG. 2A
GKLVMVGSGIKSISHMTLETVSHIEQADKVFYCVADPGTELFVKSKAKWSFDLYTLYDNDKNRYITYVQMAELCLQAARDGFFSVGVFYGHPGVFVSPSHRAIGIAKREGIEAYMLPGISAEDCLFADLGVDPSFTGCQTYEATDLLLRDRPISPYSHLIVWQVGVVGDTGFNFGGFTQTKFQVLVDRLEEVYGSDHRLIHYFASTLSHGPAHIEPLRISDLRKPEVEKRMNGISTFYVPQIG

SEQ ID NO: 129 - protein, 241 aa, Artificial Sequence
  Synthetic
  MISC_FEATURE (1)..(241): Bacterial alpha-N-methyltransferase domain shown in FIG. 2A
GSLTIAGSGIASVAHITLETLSHIREADKVFYIVCDPATEAFIHDNAKAEAVDLTVYYDTNKARYDSYVQMAEVMLQDVRGGKDVLGIFYGHPGVFVSPSHRALAIARSEGYKAKMLPGVSAEDYLFADLEFDPSVHGCATFEATELLLREKPLNTTMHNIIWQVGAVGVDDMVFTNSKLHVLVDRLEKDFGPEHQVVHYIGAVLPGSRTVMDTFTVADLCKDDVVKQFNPSSTLYIPPRS

SEQ ID NO: 130 - protein, 242 aa, Artificial Sequence
  Synthetic
  MISC_FEATURE (1)..(242): Bacterial alpha-N-methyltransferase domain shown in FIG. 2A
GELVVVGTGIASIRQMTVEALDYIQRADKVFYATLDAVTETFIKHHAPSAEDLYQYYDTEKNRVTTYVQMAEVILSSVRKGKLTVAVFYGHPGVFVTPSHRAIYIARHEGYKAQMLPGVSAEDCLYADLGIDPASSGCSMYEASFLLNEPNRLDSRHHLIIWQVGCVGKEAMIFDNKEIYKLADYLEAEYGPDHPVIAYLAAIQPFHDSKMDKMTVQDLRDQDKVQNIPITAGTTLYVPPKK

SEQ ID NO: 131 - protein, 240 aa, Artificial Sequence
  Synthetic
  MISC_FEATURE (1)..(240): Bacterial alpha-N-methyltransferase domain shown in FIG. 2A
GSLIIAGSGISSVAHLTLETVSHLKNADNVFYLVGDPVTEAFIQENNKSTTNLVAHYATSKHRYQTYVEMAEVMLREVRAGHSVFGIFYGHPGVLTTPAHRALTLARQEGYEARMLPGVSSVDYMFADLELEPGQHGCMIHEATDLLARDRRLDPSVHNIILQPSRVGSATLEKEASKFQLLVDRLVRDFGPDHKIVHYSGAVLPQSSSAMVVFVIENLRNEQLANQIRSTSILYIPPRD

SEQ ID NO: 132 - protein, 245 aa, Artificial Sequence
  Synthetic
  MISC_FEATURE (1)..(245): Bacterial alpha-N-methyltransferase domain shown in FIG. 2A
GTLTIAGSGIASIRHITLETLSYIKESDKIYYLVADPATEAFIIENANGSCVSLYGLYGIDKIRYDTYVQMSEVLLRDVRAGFDVLGIFYGHPGVFVSPTQRAMSIALEEGFQARMLPGVSAEDYLFADLRVDPCMFGCAAYEATELLYRKRRLNPTMQNIIWQVGKRFTIIKLTSPDTQNSKFGLLVDHLEEDYGPDHKVVHYIGAVLPQATTVIQPYTISELRKPEVASQIRACSTFYIPPRD

SEQ ID NO: 133 - protein, 241 aa, Artificial Sequence
  Synthetic
  MISC_FEATURE (1)..(241): Bacterial alpha-N-methyltransferase domain shown in FIG. 2A
GSLTIAGSGIASIGHITLETLSYIEQADKVYYAVADPATEAFIQDKSKVECFDLTVYYDKDKIRFETYIQMSEVMLRDVRAGHSVLGIFYGHPGVFVCPSHRAIAIALSEGYKARMLPGISAEDYMFSDIGFDPALPGCTTQEATHLLLHNKKLDPSMHNIIWQVGGVGADTMNFDNRQFHQLVDCLERDFGSSHKVVHYIGAVMPQSTTIMDEFSIADLRKEEVVKQFTTWSTFYIPPRD

SEQ ID NO: 134 - protein, 241 aa, Artificial Sequence
  Sythetic
  MISC_FEATURE (1)..(241): Bacterial alpha-N-methyltransferase domain shown in FIG. 2A
GSLTIAGSGIASVAHITLEVLSYLQEADKIYYAIVDPVTEAFIQDKSKGRCFDLRVYYDKDKMRSETYVQMSEVMLRDVRSGYNVLAIFYGHPGVFVCPTHRAISIARSEGYTAKMLPGVSAEDYMFSDIGFDPAVPGCMTQEATSLLIYNKQLDPSVHNIIWQVGSVGVDNMVFDNKQFHLLVDHLERDFGSIHKVIHYVGAIMPQSATVMDEYTISDLRKEDVVKKFTTTSTLYIPPRE

SEQ ID NO: 135 - protein, 242 aa, Artificial Sequence
  Synthetic
  MISC_FEATURE (1)..(242): Bacterial alpha-N-methyltransferase domain shown in FIG. 2A
GELVVVGTGIASLRQLTVEALDYIQRADVVFYATLDAVTEAFIKQHAKAAENLYQYYDTEKNRNATYTQMAETILASVRKGNMTVAVFYGHPGVFVTPSHRAIYIARQEGYKAKMLPGVSAEDCLYADLDIDPASSGCSMYEASFLLLEPDRLDSRHHLIIWQVGCVGKEAMVFDNKELYKLADYLEAEYGPKHPAIAYLAAIQPFNDSKMDHMTVEDLRDPEKVRSIPINAGTTLYVPPKK

SEQ ID NO: 136 - protein, 243 aa, Artificial Sequence
  Synthetic
  MISC_FEATURE (1)..(243): Bacterial alpha-N-methyltransferase domain shown in FIG. 2A
GSLTIVGSGFRSIIQFTTEALMHIEAAEKLYYCVLDAATRGFIKAKNSNSVDLYECYSNTKPRYETYIQMTEAMLRSVRDGLKATVVLYGHPGVFIHPSHRAIAIARSEGYDAWMLLGISVEDYLFADLLIDPSNPGTQTVEATEILLKERPLLTSSHVIIYQVGCIGNFTFNFSGIKNDKFDALVDRLIQEYGPDHPLVNYQAAISPLSEASIGRHIVSDLRKAEVQESVTGASTFYIPPKT

SEQ ID NO: 137 - protein, 245 aa, Artificial Sequence
  Synthetic
  MISC_FEATURE (1)..(245): Bacterial alpha-N-methyltransferase domain shown in FIG. 2A
GQLTIVGSGIASINHMTLQAVACIETADVVCYVVADGATEAFIRKKNENCIDLYPLYSETKERTDTYIQMAEFMLNHVRAGKNVVGVFYGHPGVFVCPTHRAIYIARNEGYRAVMLPGLSAEDCLYADLGIDPSTVGCITYEATDMLVYNRPLNSSSHLVLYQVGIVGKADFKFAYDPKENHHFGKLIDRLELEYGPDHTVVHYIAPIFPTEEPVMERFTIGQLKLKENSDKIATISTFYLPPKA

SEQ ID NO: 138 - protein, 245 aa, Artificial Sequence
  Synthetic
  MISC_FEATURE (1)..(245): Bacterial alpha-N-methyltransferase domain shown in FIG. 2A
GQLTIVGSGIASISHLTLQAVSAIENADIVCYVVADGATEAFIRKKNPNSLDLYHLYGEDKQRTDTYIQMAEFMLIRVRQGQNVVGVFYGHPGVFVCPTHRALYIARSEGYKARMLPGLSAEDCLFADLGIDPSSVGCVTYEATDLLVFKRPINPASHLVLYQVGIVGKSNFKFDYTSDENIHFTKLLDRLEEAYGPEHSVTHYIAPLFPTEDPIAEEYTIAQLRLPEIRDKIHTISTFYVPPKT

SEQ ID NO: 139 - protein, 243 aa, Artificial Sequence
  Synthetic
  MISC_FEATURE (1)..(243): Bacterial alpha-N-methyltransferase domain shown in FIG. 2A
GTLTIAGSGIASIGHITLETLSHIQGADKIHYAVTDPATEAFILEKSKDSSSCFDLGIYYDKNKMRYETYVQMCEVMLRDVRGGHNVLGIFYGHPGVFVSPTHRAIALARDEGYTAKMLPGISAEDYMFSDLGFDPAFPGCMTQEATILLVRGRKLDPSVHNIIWQVGGVGVDTMVFDNANFYILVDRLEEDLGPDHKVVHYIGAVLPQSTAVIDEFTVAGLRKEEVVKQITTVSTFYLPPRT

SEQ ID NO: 140 - protein, 243 aa, Artificial Sequence
  Synthetic
  MISC_FEATURE (1)..(243): Bacterial alpha-N-methyltransferase domain shown in FIG. 2A
GSLTLAGAGVTSIGHLTLQTVSAIENADIVCYILNDPVTEAFIIKKNPNVYDLYQLYDDGKPRIETYHQMVEVLMSKVRSGQDVVGLFTGHPGVVNTPAAQAFKIARQEGYTARMLPGITTNDALLADVVADPALGGAMAYEATDFLNNNRVLHPEMNVFIQQVGVVGNKHFNFMEMRSSLLDKLIDRLEETYGGEKEIIHYIAPMLPIDKPVMQKMTVSDLKKPEYKAKIVPSSTFYITPNE

SEQ ID NO: 141 - protein, 243 aa, Artificial Sequence
  Synthetic
  MISC_FEATURE (1)..(243): Bacterial alpha-N-methyltransferase domain shown in FIG. 2A
GSLTIVGTGIESIGQITLQAISHIETASKVFYCVVDPATEAFIRTKNKNCFDLYPYYDNGKHRMDTYIQMAEVMLKEVRNGLDVVGVFYGHPGVFVSPSHRALAIAESEGYKARMLPGVSAEDCLFADLRIDPSHPGCMTYEASDFLIRERPVNIHSHLVLWQVGCVGVADFNSGGFKNTKFDVLVDRLEQEYGADHPVVHYMASILPYEDPVTDKFTVSQFRDPQIAKRICGISTFYIPPKE

SEQ ID NO: 142 - protein, 243 aa, Artificial Sequence
  Synthetic
  MISC_FEATURE (1)..(243): Bacterial alpha-N-methyltransferase domain shown in FIG. 2A
GSLIVVGTGIESIGQMTLQALSYIEAASKVFYCVIDPATEAFILTKNKNCVDLYQYYDNGKSRMDTYTQMAELMLKEVRNGLDVVGVFYGHPGVFVNPSHRALAIARSEGYQARMLPGVSAEDCLFADLCIDPSNPGCLTYEASDFLIRERPVNVHSHLILFQVGCVGIADFNFSGFDNSKFTILVDRLEQEYGPDHTVVHYIAAMMPHQDPVTDKFTIGQLREPEIAKRVGGVSTFYIPPKA

SEQ ID NO: 143 - protein, 241 aa, Artificial Sequence
  Synthetic
  MISC_FEATURE (1)..(241): Bacterial alpha-N-methyltransferase domain shown in FIG. 2A
GSLTIAGTGIASIKHITLETLSYIKEAEKVYYLVADPATEAFIQDNASGTCFNLHVFYDTNKHRYDSYVQMAEVMLLDVRAGHSVLGIFYGHPGVFVSPSHRAIAIAREEGFKAHMLPGISAEDYMFADIGFDPATHGCVSYEATELLVRDKPLLPSSHNIIWQVGAIGANAMVFDNGKFNILVDRLEQVFGPDHKVVHYIGAVLPQSTSTIEAYTISDLRKGDVVEKFSTTSTLYVPPSV

SEQ ID NO: 144 - protein, 241 aa, Artificial Sequence
  Synthetic
  MISC_FEATURE (1)..(241): Bacterial alpha-N-methyltransferase domain shown in FIG. 2A
GSLTIAGSGIASIKHMTLETVSHIKEAEKVYYIVTDPATEAYIKDNAVGACFDLRVFYDTNKPRYESYVQMSEVMLRDVRVGHSVLGIFYGHPGVFVSPSHRAIAIAKEEGFQARMLPGISAEDYLFADIGFDPAAHGCMSYEATELLVRNKPLNTSTHNIIWQVGALGAEAMVFDNAKFSLLVDRLEQDYGSDHKVVHYIGAILPQADPTVEAYIVADLRKEDVVKQFNAISTLYIPPRV

SEQ ID NO: 145 - protein, 241 aa, Artificial Sequence
  Synthetic
  MISC_FEATURE (1)..(241): Bacterial alpha-N-methyltransferase domain shown in FIG. 2A
GSLTIAGTGIASIKHITLETLSYIKEAEKVYYLVADPATEAFIHDNASGTCFNLHVFYDTNKLRYDSYVQMAEVMLRDVRAGNSVLGLFYGHPGVFVSPSHRAIAVAREEGFKAQTLPGISAEDYMFADIGFDPASHGCVSYEATDLLARDKPLLPSSHNIIWQVGAIGANAMVFDNGKFNVLVDRLERDFGPNHKVVHYIGAVLPQSTSKVEQYTVADLRKDYVVKTFTTTSTLYVPPCV

SEQ ID NO: 146 - protein, 243 aa, Artificial Sequence
  Synthetic
  MISC_FEATURE (1)..(243): Bacterial alpha-N-methyltransferase domain shown in FIG. 2A
GGLVVVGSGIRSVSQLTLEAVMHIEKADTVLYCVCDPSTEGFIKRKNKNAIDIYGYYSDLKERPDAFVQMAEVILREVRKGINVVAVFYGHPGIFVHPSRRALAIAKKEGYAARMLPGISAEDCLFADLLVNPSFPGAQLVEASDIVYRARPLATSCHVVIFQAACFGHWKYNFTAFENGKFDHLVNRLQKDYGPDHPIVSYMAAVSPLEDPVINRHTISDLYKADVKKEITPNCTLYIPPKD

SEQ ID NO: 147 - protein, 241 aa, Artificial Sequence
  Synthetic
  MISC_FEATURE (1)..(241): Bacterial alpha-N-methyltransferase domain shown in FIG. 2A
GSLTIAGSGIASVGHITLETLAYIKESHKVFYLVCDPVTEAFIQENGKGPCINLSIYYDSQKSRYDSYLQMCEVMLRDVRNGLDVLGVFYGHPGVFVSPSHRAIALAREEGFNAKMLAGVSAEDCLFADLEFDPASFGCMTCEASELLIRNRPLNPYIHNVIWQVGSVGVTDMTFNNNKFPILIDRLEKDFGPNHTVIHYVGRVIPQSVSKIETFTIADLRKEEVMNHFDAISTLYVPPRD

SEQ ID NO: 148 - protein, 241 aa, Artificial Sequence
  Synthetic
  MISC_FEATURE (1)..(241): Bacterial alpha-N-methyltransferase domain shown in FIG. 2A
GSLTIAGSGIASIRHMTLETLSAIKSADKVYYTVCDPATEAFIQDNATGSCSDLTVYYDKEKSRYDTYVQMCEVMLREVRAGHNVLGVFYGHPGVFVSPSHRAIAIARAEGYKAEMLAGVSAEDYMFADLGFDPAAHGCVTYEATEMLLRKKQLNPATHNIIWQVGGVGVSNMIFDNARFHLLVDRLEDTFGPDHQVVHYIGAVLPLSVKTMETYTIADLRKEDVVAQFNPTSTLYIPPRD

SEQ ID NO: 149 - protein, 243 aa, Artificial Sequence
  Synthetic
  MISC_FEATURE (1)..(243): Bacterial alpha-N-methyltransferase domain shown in FIG. 2A
GSLTIVGTGIESIGQMTLQTLSYIEAADKVFYCVIDPATEAFILTKNKDCVDLYQYYDNGKSRMDTYTQMSEVMLREVRKGLDVVGVFYGHPGVFVNPSLRALAIAKSEGFKARMLPGVSAEDCLYADLCIDPSNPGCLTYEASDFLIRERPTNIYSHFILFQVGCVGIADFNFTGFENSKFGILVDRLEKEYGAEHPVVHYIAAMLPHEDPVTDQWTIGQLREPEFYKRVGGVSTFYIPPKE

SEQ ID NO: 150 - protein, 241 aa, Artificial Sequence
  Synthetic
  MISC_FEATURE (1)..(241): Bacterial alpha-N-methyltransferase domain shown in FIG. 2A
GSLTIAGSGIASIRHITLETLSHIERADKVYYLVADPATEAFIQDKSKGDYVDLAIYYDKDKNRYESYVQMSEVILNDVRAGYNVLGVFYGHPGVFVSPSHRTVAIARDEGYRVNMLPGVSAQDYMFSDIGFDPAIPGCTIQEASTILFLDKRLDPTVHNIIGQVGCVGVGTMAFDNRQFHLLVDHLEKDFGPEHKVVHYIGAVLPQSATVKDEFKIADLRKDDVVKQISTISTFYIPPRQ

SEQ ID NO: 151 - protein, 241 aa, Artificial Sequence
  Synthetic
  MISC_FEATURE (1)..(241): Bacterial alpha-N-methyltransferase domain shown in FIG. 2A
GSLTIAGSGIASIGHITLETLALIKEADKIFYAVTDPATECYIQENSRGDHFDLTTFYDTNKKRYESYVQMSEVMLREVRAGRNVLGIFYGHPGVFVAPSHRAIAIAREEGFQAKMLPGISAEDYMFADLGFDPSTQGCMTQEATELLVRNKKLDPSVHNIIWQVGSVGVDTMVFDNGKFHLLVERLEKDFGLDHKIQHYIGAILPQSVTVKDAFAIRDLRKEEVLKQFTTTSTFYIPPRA

SEQ ID NO: 152 - protein, 242 aa, Artificial Sequence
  Synthetic
  MISC_FEATURE (1)..(242): Bacterial alpha-N-methyltransferase domain shown in FIG. 2A
GELVVVGTGIASLRQMTVEALDYIQRADMVFYVVLDAMTECFIQTHAKKHHDLYQYYDKNKPRNASYVQMAELMVQSVRDGNLTVAVYYGHPGVFVFPTHRAIHIAREEGYKAKMLPGVSAEDCLYADLGIDPGTTGCSMFEATYLLNEPDRLDPRNHVIIWQPGCVGKSTMVFDNSEIHELADYLEKTYGPEYPIIAYLAAVRPFNDPQIDKLMVKDLRDLEKLKAIPFNAATTLYIPPKT

SEQ ID NO: 153 - protein, 243 aa, Artificial Sequence
  Synthetic
  MISC_FEATURE (1)..(243)
GSLTIVGTGIESIGQMTLQALSYIEAAAKVFYCVIDPATEAFILTKNKNCVDLYQYYDNGKSRLNTYTQMSELMVREVRKGLDVVGVFYGHPGVFVNPSHRALAIAKSEGYRARMLPGVSAEDCLFADLCIDPSNPGCLTYEASDFLIRDRPVSIHSHLVLFQVGCVGIADFNFTGFDNNKFGVLVDRLEQEYGAEHPVVHYIAAMMPHQDPVTDKYTVAQLREPEIAKRVGGVSTFYIPPKA

SEQ ID NO: 154 - protein, 241 aa, Artificial Sequence
  Synthetic
  MISC_FEATURE (1)..(241): Bacterial alpha-N-methyltransferase domain shown in FIG. 2A
GSLTIAGSGIASVRHMTLETLAHVQEADIVFYVVADPVTEAYIKKNARGPCKDLEVLFDKDKVRYDTYVQMAETMLNAVREGQKVLGIFYGHPGVFVSPSRRALSIARKEGYQAKMLPGISSEDYMFADLEFDPAVHGCCAYEATQLLLREVSLDTAMSNIIWQVGGVGVSKIDFENSKVKLLVDRLEKDFGPDHHVVHYIGAVLPQSATVQDVLKISDLRKEEIVAQFNSCSTLYVPPLT

SEQ ID NO: 155 - protein, 241 aa, Artificial Sequence
  Synthetic
  MISC_FEATURE (1)..(241): Bacterial alpha-N-methyltransferase domain shown in FIG. 2A
GSLTIAGSGIASVAHITLETVAYLAEADSVFYIVADPVTEAFIHKNAKVPCQDLHVFYDKDKSRYDTYVQMAETMLNSVRAGEKVLGIFYGHPGVFVSPSRRALAIAREEGYEAKMLPGVSAEDYMFADLEFDPATHGCCAYEATHILLKNIPLDTSINNIIWQVGGVGVTKIDFENSKFKFLVDRLEKDFGLDHKVVHYIGAVLPQSATVKEVYTISDLRKPEVATQFNACSTLYVPPRK

SEQ ID NO: 156 - protein, 241 aa, Artificial Sequence
  Synthetic
  MISC_FEATURE (1)..(241): Bacterial alpha-N-methyltransferase domain shown in FIG. 2A
GTLVIAGSGIASIAHITLETLSHIKESDRVYYIVGDPATEAFIQDNASGTCFDLTIFYDTNKVRYDSYVQMCEVMLRDVRAGHTVLGVFYGHPGVFVSPSHRAIAIARDEGYKARMLPGVSAEDYLFADLGFDPATHGCTSYEATDLLVRNKPLNASTHNIIWQVGGVGVGTMVFDNAKFHLLVDRLEKDFGPSHTVVHYIGAVLPQSITTMDKLTIADLRKDAVVKQFNPTSTFYIPPRD

SEQ ID NO: 157 - protein, 241 aa, Artificial Sequence
  Synthetic
  MISC_FEATURE (1)..(241): Bacterial alpha-N-methyltransferase domain shown in FIG. 2A
GTLTIAGSGIASVAHITLETLSYIKESEKIFYLVCDPVTEAYIQDNTTADCFDLSVFYGKNKGRHDSYIQMCEVMLKAVRAGHDVLGVFYGHPGVFVSPSHRAIAVARQEGYKAKMLPGISAEDYMFADLEFDPSLSGCKTCEATEILLRDKPLDPSIQNIIWQVGSVGVVDMEFEKSKFQLLVDRLEKDFGPGHKVVHYIGAVLPQSTTTMDTFTIADLRKEDVAKQFGTISTLYVPPRD

SEQ ID NO: 158 - protein, 242 aa, Artificial Sequence
  Synthetic
  MISC_FEATURE (1)..(242): Bacterial alpha-N-methyltransferase domain shown in FIG. 2A
GTLTIAGSGIACVAHITLETLSYIKESDKLFYLVCDPVTEAFIQDNATGDCFDLSVFYDKNKSRYDSYIQMCEVMLKAVRAGHHVLGVFYGHPGVLVSPSYRAIAVAREEGYKARMLPGISAEDYLFADLEFDPCFPSGCNTYEATELLLRDRSLDPSIHNIIWQVGSVGVTDMEFEKSKLNLLVDRLENDFGPDHKVVHYIGAVLPQSTTTMDTFAVSDLHKEDVAKQFGTISTLYIPPRD

SEQ ID NO: 159 - protein, 241 aa, Artificial Sequence
  Synthetic
  MISC_FEATURE (1)..(241): Bacterial alpha-N-methyltransferase domain shown in FIG. 2A
GTLTIAGSGIASIAHITLETLSYIKESDKLFYLVCDPVTEAFIQDNATGDFFDLSVFYDKNKSRYDSYIQMCEIMLRAVRAGHSVLGIFYGHPGVFVSPSHRAIAVAREEGYKARMLPGVSAEDYMFADLEFDPSQSTCNTYEATELLLRDRPLDPAIQNIIWQVGSVGVVDMEFEKSKFHLLVDRLEQDFGPDHKVVHYIGAVLPQSTTTMDIFTISDLRKENVAKQFGTISTLYIPPRD

SEQ ID NO: 160 - protein, 240 aa, Artificial Sequence
  Synthetic
  MISC_FEATURE (1)..(240): Bacterial alpha-N-methyltransferase domain shown in FIG. 2A
GTLTIAGSGIASIGHITLETLSYIQEADKIHYAVADPATEAFILDKSKDSSHCFDLTVYYDTNKMRYETYVQMCEVMLRDVRGGYNVLGIFYGHPGVFVSPSHRAIAIARDEGYIAKMLPGVSAEDYMFSDIGFDPAVPGCMSQEATGLLVCKKKLDPSIHNIIWQVGSVGVDTMNREFHILVDRLEEDFGLDHKVVHYIGAVLPQSTTVMDEFTIADLRKEEVVKQITTTSTFYLPPRS

SEQ ID NO: 161 - protein, 242 aa, Artificial Sequence
  Synthetic
  MISC_FEATURE (1)..(242): Bacterial alpha-N-methyltransferase domain shown in FIG. 2A
GSLTIAGTGIATLAHMTLETVSHIKEADKVYYIVTDPVTQAFIEENAKGPTFDLSVYYDADKYRYTSYVQMAEVMLNAVREGCNVLGLFYGHPGIFVSPSHRALAIAREEGYEARMLPGVSAEDYMFADLGLDPALPGCVCYEATNFLIRNKPLNPATHNILWQVGAVGITAMDFENSKFSLLVDRLERDLGPNHKVVHYVGAVLPQSATIMETYTIAELRKPEVIKRISTTSSTFYIPPRD

SEQ ID NO: 162 - protein, 242 aa, Artificial Sequence
  Synthetic
  MISC_FEATURE (1)..(242): Bacterial alpha-N-methyltransferase domain shown in FIG. 2A
GSLIIAGSGIASVAHFTLETVSHLKNADKVFYLVNDPVTEAFIQENNPDTFDLVTFYSETKPRYHSYVEMAEIMLKEVRAGHKVLGIFYGHPGVFVHPSRRALFIARQENYEARMLPGISSEDYMFADLELDPAEFGCMTCEATELIARNRPLNTSVHNIIWQAGIVGVSTLEYQESKFQLLVDRLERDFGPEHKVVHYVGAIRMTPQAQSAMVVYSIQELRNPAVANFINSGSTLYVPPRL

SEQ ID NO: 163 - protein, 243 aa, Artificial Sequence
  Synthetic
  MISC_FEATURE (1)..(243): Bacterial alpha-N-methyltransferase domain shown in FIG. 2A
GRLVMVGSGIKSIAHLTLEAIGHIEQADKVFFVVADMTTAAFIHSRNANAVDMYNLYDIGKPRYHTYVQMAERMLREVRNGFYVVGVFYGHPGIFVNPSHRAIAIARQEGHQAFMLPGISAEACLFADVGIDPSTSGCQTIEATDLLLRNRPINTGSHLIIFQVGIVGDSGFHPQGFKNTKLHVLLEKLTEVYGSGHRLVHYIAPSMATVEPTIDFLTLGALKKSRNARRVTGISTFYIPPKH

SEQ ID NO: 164 - protein, 243 aa, Artificial Sequence
  Synthetic
  MISC_FEATURE (1)..(243): Bacterial alpha-N-methyltransferase domain shown in FIG. 2A
GSLFIVGSGIRSIAQLTLEAIMHIENADKVFYVVCDPVTEGFIKEKNPNAVDLYEYYSNTKLRNETYIQMAEIMLREVRSGLRVVGVFYGHPGNFVSPTRRALAIARDEGYVAKMLPGISADDCLFADLLIDPCYPGLQTVEATDVLVRNRPLQTTSHVVIYQVGVICKSGFDFYSIENDKFDHFVTRLQEDYGPNHPVVNYVAAVSPLAEPTIQRHTISELFKDSVKASISGVSTFYIPPKE

SEQ ID NO: 165 - protein, 243 aa, Artificial Sequence
  Synthetic
  MISC_FEATURE (1)..(243): Bacterial alpha-N-methyltransferase domain shown in FIG. 2A
GKLVIVGSGIRSISQFTLEAVAHIEHADKVFYCVADPGTDAFIERHNKNAVDLYNLYGDGKPRHQTYTQMAEVILQEVRKGFSVVGVFYGHPGVFVNPAHRAVSIAASEGYEATMLPGVSAEDCLYADLLIDPSRPGCQTLEATDVLLRKRPIAKDCHVIIFQVGAVGDLGFNFKGFKNTKFEILVQHLLEVYGPDHSVVHYIASQLTFAAPIRDRYAIQDLVKPEVAKRITGISTFYLPPKD

SEQ ID NO: 166 - protein, 203 aa, Artificial Sequence
  Synthetic
  MISC_FEATURE (1)..(203): Archaeal alpha-N-methyltransferase domain shown in FIG. 2A
MDKLYEDRIEEMGTEQYDLRNHYTEQEDRAETYEKMAETVLQAAEEVDEPVTLGLYGHPMVFVSPSQKVINRAEERGMMVEVRPGISSMDCLYVDLEFDPGRSGVQMFEATDLMLREWELNPEVPAMIWQIGGVESTIHHESVEDKPERYSRIREYLQQFYPDDHTVVSAKTPIYPTTEPQQYEFQLDEFESMADSIDHLQTL

SEQ ID NO: 167 - protein, 240 aa, Artificial Sequence
  Synthetic
  MISC_FEATURE (1)..(240): Archaeal alpha-N-methyltransferase domain shown in FIG. 2A
DIYVIGTGMVGTRQFTQEAISALEQCERVYLVHFQKSVKKYLEEFTDQVHVLTDEYNEGEKRDNTYARMAQQVLDGAEESEGPVVFALYGHPMVFVSPSRWVIDEAPERGLEVEVQPGISSMDCLYSDIAFDPAKNGVQMFEASDLLLREFDLNPDIPAMIWQVGMLESALYTTNSSEPERFTRFREYLERFYPPDHSVSLLQTATYPISNSRQIEFEIKDFEKMADEINAIQTLYIPPA

SEQ ID NO: 168 - protein, 241 aa, Artificial Sequence
  Synthetic
  MISC_FEATURE (1)..(241): Fungal alpha-N-methyltransferase domain shown in FIG. 2A
GSLVCVGTGLQLAGQISVLSRSYIEHADIVFSLLPDGFSQRWLTKLNPNVINLQQFYAQNGEVKNRRDTYEQMVNAILDAVRAGKKTVCALYGHPGVFACVSHMAITRAKAEGFSAKMEPGISAEACLWADLGIDPGNSGHQSFEASQFMFFNHVPDPTTHLLLWQIAIAGEHTLTQFHTSSDRLQILVEQLNQWYPLDHEVVIYEAANLPIQAPRIERLPLANLPQAHLMPISTLLIPPA

SEQ ID NO: 169 - protein, 238 aa, Artificial Sequence
  Synthetic
  MISC_FEATURE (1)..(238): Fungal alpha-N-methyltransferase domain shown in FIG. 2A
GSLQVVGSGILPISDMSLGAQAQIMAADIVLYCVADPATELRIHELNPRSTSLYGLYGNGVPRIETYRAMVDAMLEQVRRGRRTCAVFYGHPGVFAWSTHEAIRRARSEGYRAEMGSAVSAQDSLFADLGLDPSSFGLMTLDATDFLIRNRSLDPSMHVLLWQAECAGDDGFNFAGYRRHNFPILIERVSGFYPPDHPVVIYDASTFGHLPPVIQLKRLDTIKASDLSGISTLYLPPA

SEQ ID NO: 170 - protein, 240 aa, Artificial Sequence
  Synthetic
  MISC_FEATURE (1)..(240): Fungal alpha-N-methyltransferase domain shown in FIG. 2A
GRLVVVGSGIRPITDLSMGARDQIVAADVVVYCVADPVTELYLHELNPNAHSLYGLYGNAKPRIETYKEMVEAMLAPVREGLRVCAVFYGHPGIFAWATHEAIRRARDAGLRAEMAPAISSQDSLFADLGIDPSSAGLQTVDATDLLIRGRELNPSMHVLVWQPECVGDDGFNFAGYERHNFPVLVEYLRRTYAGDHTVVIYDASTYPHLPPSSARRRSTRSLPGTSPGSRRSTCPRPSP

SEQ ID NO: 171 - protein, 238 aa, Artificial Sequence
  Synthetic
  MISC_FEATURE (1)..(238): Fungal alpha-N-methyltransferase domain shown in FIG. 2A
GRLVVVGSGIRPISDLTLGARDQIIAADVVVYCVADPITELQIHELNPNAHSLYGLYGNGKPRIDTYREMVEAMLTPLREGKRVCAVFYGHPGIFAWATHESIRQARAAGLRAEMAPAISSQDSLFADLGIDPSSAGLQTVDATDLLIRRRQLNPTMHVLIWQAECVGDDGFNFAGYERHNFPILIEYLQQTYPSDHTVVIYDASTYPHLPPTIRSTTLDVVDAQDLSGISTLYLPPA

SEQ ID NO: 172 - protein, 238 aa, Artificial Sequence
  Synthetic
  MISC_FEATURE (1)..(238): Fungal alpha-N-methyltransferase domain shown in FIG. 2A
GQLVIVGSGIRAIADLTFDAEAQIRTAQKVLYCVADPVIERRLHLLNSTAESLYGLYGNNKPRIETYHAMVDALLAPVRAGLRVCGVFYGHPGSFAWPTHQAIRVARRDGFKAEMHASVSADASLFADLGIDPSQPGCHSLEATEFLIHRRVPDTSSHLLVWQAECVGDPGFNFAGYKRHNFDILINQLQKFYPADHPVFIYEAASLPQGRPKIIKSTLDTIRKEDLTGISTLYLPPS

SEQ ID NO: 173 - protein, 238 aa, Artificial Sequence
  Synthetic
GSLVCVGTGMMVGAHLSPICQSHIEQADVVFVCVAEHYMEAWITSLNKNTVNLQTFYGEGKDRHDTYAQMTSAMLERVEKGEKVVGAFYGHPGVFAKVPHDAIAIAKSRGFDAYMLPGISAESCLYADLGIDPGALGCQHYEANQFLLYKREVDTAAYLVLWQVGVAGDFSSAVFTSSSLQRKKLTDKLLSIYQANDSAILYEAATLPIDEFRATTIAIGELHNHEINQHSTLVIPPV

SEQ ID NO: 174 - protein, 238 aa, Artificial Sequence
  Synthetic
  MISC_FEATURE (1)..(238): Fungal alpha-N-methyltransferase domain shown in FIG. 2A
GSIACVGMGITLGSHLTPLSRSHIEQADVVFAALSDHVVELWLKRLNPDVRSLQPYYKPGKSRMRTYREWVQLMIAEVRAGKSVCAVFYGHPGIFAWSTHKVVELARAEGYKAHMEAGISAEDCLYADLGIDPGRVGCQHLEASQLLFFERNLDTTSHVILWQLGVVGDRSLGRFRTPDAYRELLVERLRDDYAEDHEVIVYRAPTLPIESAHIQRMPLKDFVNAQFTGQETLVLPPA

SEQ ID NO: 175 - protein, 238 aa, Artificial Sequence
  Synthetic
  MISC_FEATURE (1)..(238): Fungal alpha-N-methyltransferase domain shown in FIG. 2A
GSLVCVGLGMTLGSHLGPLARSHIEQADVVFAGLSDGIVELWLNKMHADVRSLQPYYEEGKSRMLTYRQMVEAILAEVRAGKRVCGVFYGHPGVFAWAPHKSIEIARSEGYSAHMEPGISAEDCLYADLGIDPGRFGCQHYEASQLLFYQRRIDPTAYLVLWQVGLVGDQSLARFSTGPAYRQVLVDVLARDYPLDHELIIYRSATLPIQQPRIERMALGDLPQADIGVPDTVVVPPA

SEQ ID NO: 176 - protein, 238 aa, Artificial Sequence
  Synthetic
  MISC_FEATURE (1)..(238): Fungal alpha-N-methyltransferase domain shown in FIG. 2A
GSLACVGIGMTLGAHICPLSKSYIEQADVVFSAVSHGITELWLQEMHSDVRSLQEYYQEGKSRLITYNEMVDAMMTEVRAGKKVVGAFYGHPGVFAQAPHKSIAMAKAEGFAAKMIPGISAEDCLIADLGIDPGRFGCQQFEASQFMFYKRQYDPSCYLILWQIGLAGDRSLAKFSTGAAHRQVLIELLSEVYPLDHQVILYEAAVLPIDNIRIETLSLSELVTAEIHMHTTLVIPPS

SEQ ID NO: 177 - protein, 238 aa, Artificial Sequence
  Synthetic
  MISC_FEATURE (1)..(238): Fungal alpha-N-methyltransferase domain shown in FIG. 2A
GSLACVGIGMTLGAHICPLAKSYIEQADVVFSGVSNGIVELWLQEMHSDVRSLQVYYKEGKSRHITYREMVDAILTEVRAGKKVVGAFYGHPGVFAHAPHKSIELAKAEGFDAVMVPGISAEDCLIADLAIDPGKFGCQQFEASQFMFYKRRFDPSSYLVLWQIGLAGDKSMAKFATGAAHRQVLIELLSTEYPLDHQVILYEAAVLPIDTVRKQTLTLAELADADIYMHTTLVIPPS

SEQ ID NO: 178 - protein, 238 aa, Artificial Sequence
  Synthetic
  MISC_FEATURE (1)..(238): Fungal alpha-N-methyltransferase domain shown in FIG. 2A
GSLVCVGVGMTMGSHLTPLSRNYIETADVVFSLMSDGIVEQWVETMNPDVRSLQPFYQEGTSRLISYQNMINAVLDEVRQGKNVVGAFYGHPGVFAMVTHKLIAQSKKEGFYCHMEAGISAEDCLIADLGIDPGRLGCQQYEASQFMFYRRTIDTAAYLILWQPGIAGDQSLARFATAESFRAVLVELLCEYYAKDHQVILYEAKTLAFNNPRIDTLPLGELAKATIHLHTTLVIPPS

SEQ ID NO: 179 - protein, 238 aa, Artificial Sequence
  Synthetic
  MISC_FEATURE (1)..(238): Fungal alpha-N-methyltransferase domain shown in FIG. 2A
GNLVCVGTGILLGGHLSPLAQNLIEQADVVFSGMSDGFTELWIEGLAKDVRSLQQHYADGKSRNLTYNEMVDAMLCEVRLGKKVVGAFYGHPGIFAKAPHEAIATARAEGFEARMIPGISAEDCLYADLGLDPGKTGCQHFETTQLMLYQRRIDPSALLVLWQPALAGDLTLGIRPTGSAERELLVELLSQDYPLNHECILYEAATTALHSTRIERLPLSALPQARLELHTTLVLPPS

SEQ ID NO: 180 - protein, 238 aa, Artificial Sequence
  Synthetic
  MISC_FEATURE (1)..(238): Fungal alpha-N-methyltransferase domain shown in FIG. 2A
GSLTCVGVGMMLGGHLSPIAHSHITQADVVFSGVSDGFVEQWLSGLNADVRSLQVHYGEGKSRNISYGEMVQVMLAEVRLGKKVVGAFYGHPGIFVKSTHEAIAKAKEEGFAAKMIPGISAESCLYADLGIDPGKVGCQHFETTQFMLYHRQLDPSAHLILWQPGLAGDLTYGIKPTGRAERQLLVELLSKDYPLEHECILYEAATMPLQSGRIERLPLNHLPLAQIALHTTLVLPPS

SEQ ID NO: 181 - protein, 238 aa, Artificial Sequence
  Synthetic
  MISC_FEATURE (1)..(238): Fungal alpha-N-methyltransferase domain shown in FIG. 2A
GSITCVGVGMMLGAHLSPVSLSHIEQADIVFCGVSDPLVELWLKELNANVRSLQPCYAHGKSRHLSYREMVDTMLSEVRLGKKVVGAFYGHPGVFAKAPHQAVTIARNEGFEAQMLPGISAADCLYADLGIDPGQNGCQHFEATQFMLYHRRVDPTATLILWQIGLAGDLDMGLTITDAKNRQLLLEELYRLYSPEHSCWLYEAATTALASPRREAIKLRELVEATLALHTTLVIPPA

SEQ ID NO: 182 - protein, 241 aa, Artificial Sequence
  Synthetic
  MISC_FEATURE (1)..(241): Fungal alpha-N-methyltransferase domain shown in FIG. 2A
GNLVCVGTGLQLAGQIGALSLSYIEHADVVFSLVPDGFSERWLMSLNCDVRSLQPYYAQHDEVKNRRDTYAEMVDAILDEVRLGKLVVCALYGHPGVFACVAHWSIKQARSEGFDASMLPGISAEACLWADLGIDPGHSGHQSFEATQFMLYHHIPDPTTHLLLWQIALAGEHTLTQFSSTKDKLQILVEHLNQWYPLTHAVIIYEAANLPFQSPVIERIPLSLLPEATLTTISTLLIPPA

SEQ ID NO: 183 - protein, 241 aa, Artificial Sequence
  Synthetic
  MISC_FEATURE (1)..(241): Fungal alpha-N-methyltransferase domain shown in FIG. 2A
GSLVCVGTGLQLAGQINVMSRSYIEHADIVFSLLPDGFSQHWIQQINPNVINLQQFYAQNTEVKNRRDTYVEMVAAILEAVRAGKKTVCALYGHPGVFACVSHLAIRQAKAEGFSAKMEPGISAEACLWADLGIDPGNSGHQSFEASQFMFFNHVPDPTTHLLLWQIAIAGEHTLTQFHTTTDRLQILVEQLSQWYPMDHQVVVYEAANLPIQAPRVDYLLLSELPKAHLSPISTLLIPPA

SEQ ID NO: 184 - protein, 241 aa, Artificial Sequence
  Synthetic
  MISC_FEATURE (1)..(241): Fungal alpha-N-methyltransferase domain shown in FIG. 2A
GTLVCVGTGLNLAGQISVLSKSYIEHADVVFSLVPDGFAQHWLESLNQDVRSLQPYYAQEGEVKSRRDTYDQMVDAILEQVRLGKQVVCALYGHPGVFACVSHFAIAQAREEGYSAQMEPGISAEACLWADVGIDPGASGHQSFEASQFMFYKHTPDPTTHLLLWQIGIAGEHTLTEFHTSSDRLQVLVEQLSEWYPLEHEIVLYEAPNLPTQSPRIERVALKQLPFAQLSSITTLLIPPS

SEQ ID NO: 185 - protein, 241 aa, Artificial Sequence
  Synthetic
  MISC_FEATURE (1)..(241): Fungal alpha-N-methyltransferase domain shown in FIG. 2A
GSLVCVGTGLNLAGQISVLSKSYIENADVVFSLVPDGFAQRWLETLNSDVRSLQPYYAQGDEIKSRRDTYDQMVQAILNAVRDGKKVVCALYGHPGVFACVSHFAIEIAREEGYIAKMEPGISAEACLWADLGIDPGNSGHQSFEASQFMFYKHTPDPTTHLLLWQIGIAGEHTLTEFHTSSDRLQVLVEQLNQWYPLEHEIILYEAPNLPIQQARIDKLPLKNLPFARLTAITTLLIPPS

SEQ ID NO: 186 - protein, 241 aa, Artificial Sequence
  Synthetic
  MISC_FEATURE (1)..(241): Fungal alpha-N-methyltransferase domain shown in FIG. 2A
GSLVCVGTGLNLAGQISVLSKSYIENADVVFSLVPDGFAQRWLETLNNDVRSLQPFYAQGDEIKNRRDTYDQMVEAILEQVRAGKRVVCALYGHPGVFACVSHFAIAQALDEGYSAKMEPGISAEACLWADLGIDPGQSGHQSFEASQFMFYKHTPDPTTHLLLWQIGIAGEHTLTEFHTSSDRLQILVEQLNEWYPLEHEITIYEAPTLPIHQARVDKLLLRDLPFARLTSISTLLIPPS

SEQ ID NO: 187 - protein, 241 aa, Artificial Sequence
  Synthetic
  MISC_FEATURE (1)..(241): Fungal alpha-N-methyltransferase domain shown in FIG. 2A
GSLVFVGTGLQLAGQISVLSRSYIENADRVFSIVPDAFAEEWLISLNPNHTSLQSYYAQADEVKNRRHTYKQMVEAMLVAVRKGEQVVCALYGHPGVFACVSHLAIAQARTEGFDSYMEPGISAEACLWADLGIDPGQCGHQSFEATQFLLYQHRPDPSTHLLLWQIALAGDHTLTQFSTTSDRLQVLVDLLSQWYPLDHEVVIYEAANLPLQQPRIERLALKALPQARLTVISTLLIPPS

SEQ ID NO: 188 - protein, 241 aa, Artificial Sequence
  Synthetic
  MISC_FEATURE (1)..(241): Fungal alpha-N-methyltransferase domain shown in FIG. 2A
GSLVCVGTGLKMAGHISVISRSYIEHADKIFTLMPDVHTQQWIARLNPSLVNLQQFYAQPGEVKSRRDTYHQMVDTMMQAVRAGERVVGAFYGHPGVFACVPHMAIKQAREEGFEAHMEPGISAEACLWADMGIDPGDYGHQSFEASQFMFFKRTPDPAAHLLLWQIALAGEHTLTQFSTTSERLEILVGYLQQWYPADHQVAIYEAANLPVQQPRIDWLELQALPGTRLTAASTLLIPPS

SEQ ID NO: 189 - protein, 241 aa, Artificial Sequence; identical to SEQ ID NO: 188
  Synthetic
  MISC_FEATURE (1)..(241): Fungal alpha-N-methyltransferase domain shown in FIG. 2A

SEQ ID NO: 190 - protein, 237 aa, Artificial Sequence
  Synthetic
  MISC_FEATURE (1)..(237): Fungal alpha-N-methyltransferase domain shown in FIG. 2A
AGLVVVGSGIDFGRHASERCLSEIREAEVVFCLVNPVALAMICSFRPDAINLGVHYAPGKDRRETYREIDDRIMAEVRAGRRVCAVFYGHPGVFADVPHRVVRQARAEGIPARMEPGISAEACLYADLGLDPGRRGVQSMEATHLMVYDRQLDSAGLVLLWQVALAGDLSCTRFHAERDGLKALVDRLLCWYPPEHEVILYEAAWLPIEQPRIERLALRDLPDARYAEYTTLVIPPL

SEQ ID NO: 191 - protein, 238 aa, Artificial Sequence
  Synthetic
  MISC_FEATURE (1)..(238): Fungal alpha-N-methyltransferase domain shown in FIG. 2A
GELVFVGSGIKGTHHITPETSLHIRTAGKVLYCVADVIVERKIRLLNNNCEDLYELYADDKPRRKTYEEMVEHVLEALKHYEKVCVVFYGHPGVFVWSSFTAIQKARRDGIKAYMLPAVSSLDCMFADIGFDPSRRGCQIFEATDFLTRTRTPDTTAAVIILQLGAVGDMGFRFRGYDRRNMPVFVEYLQNFYGADHEVILYQAAQYPVCEPVIQKVAIRDIVAAKPTGITTLYIPPK

SEQ ID NO: 192 - protein, 238 aa, Artificial Sequence
  Synthetic
  MISC_FEATURE (1)..(238): Fungal alpha-N-methyltransferase domain shown in FIG. 2A
KELVFVGTGIKGAHHLTAEAILHIETADKVLYCVADVVVERKIRQLNDNCRDLYDLYGDDKLRRRTYEEMVECSMDALKEYRKVCVVFYGHPGIFVWPSFTAIRQARQAGVRAYMLPAVSSIDCMFADLGFDPSRHGCQILEATDFLSRGKKPDVNASVIILQVGAVGDMGFRFRGYEKRNMPIFVEYLAGFYGPDYEVILYEASQFPVCAPTIRKLPIRDITAANPSGITSLYIPPK

SEQ ID NO: 193 - protein, 238 aa, Artificial Sequence
  Synthetic
  MISC_FEATURE (1)..(238): Fungal alpha-N-methyltransferase domain shown in FIG. 2A
KELVFLGTGIRGASQITPEAEYHIRSADKVFYCVGDLVIERKIMNLNSTSEDLYPLYGDEKPRRETYEEMVSVCYGALKVNRKVAVIFYGHPGIFVYASHKAIRMARAEGIKAYMAPGISSLDCLFADIGIDPSRFGCQIFEATDLLVKRRKPGIDTAVIIMQVGCVGDTGFRFKGYERRNMPILQEYLSEIYGDNYEVILYEAASYPECEPRIRLIPLFNLVESSPTGITTMYIPPK

SEQ ID NO: 194 - protein, 238 aa, Artificial Sequence
  Synthetic
  MISC_FEATURE (1)..(238): Fungal alpha-N-methyltransferase domain shown in FIG. 2A
NEVIFIGTGIKGANHISAEADAYIANASKVLYCVADLAVERKILLLNQNSEDLYIYYGDNKPRRETYEQMVDRILEVIKTEKLVCVVFYGHPGIFVWPSYQAIEKARGMGYKASMLPAISSLDCLFADIGFDPSRYSCQIVEATDLLIRSRNLDVSASVIIFQIGLVGDLGYSSKGFDGRNVPILADYLSSFYGDDYEVIIYEAAQYPIFEPKIQLIKINELSKAKLTGISTMYIPPK

SEQ ID NO: 195 - protein, 238 aa, Artificial Sequence
  Synthetic
  MISC_FEATURE (1)..(238): Fungal alpha-N-methyltransferase domain shown in FIG. 2A
GSLTVVGTGIRLVTQLTPEARAAIRDAEKVFYVTDGPVQRRWFEQLNSTAESLHHLYQLGRDRLSTYEEIVERALAAARGGVRVCLVMYGHPGVLVTPAHDAIRSARAEGLSATMLPGVSAEDCLFADLGVDPGARGCQTFEATDFLVQRRRFDPRSALVLYQIAAIGVRAHTATLPNLRGLRALSAALVEHYPAEHQAIVYTAAEYPGCRAIIEPSTIAGLPSAPVQATATLYVPPL

SEQ ID NO: 196 - protein, 238 aa, Artificial Sequence
  Synthetic
  MISC_FEATURE (1)..(238): Fungal alpha-N-methyltransferase domain shown in FIG. 2A
GCLTIVGTGIQFVGQVTLAAKAWIEQADKVLYAVADPATAQWLVSVNSTAEALPYNRNNRQRKETYVQMVECILAEVHRGLNVCVVFYGHPGVFADSAHQALRRARQEGFRAQMLPGISAEDCLFADLSLDPGKRGCQSFEATDFLIRRRKFDPTSALILWQIALVGNRGFYEQGGHVRGLHVLTEVLQNYYASDSEAIVYEAAVYHPVCEPSIQRIPLEKLPEAKVTESSTLYMPPN

SEQ ID NO: 197 - protein, 238 aa, Artificial Sequence
  Synthetic
  MISC_FEATURE (1)..(238): Fungal alpha-N-methyltransferase domain shown in FIG. 2A
GSLTIVGTGIQLVSHLTLGAKAWIEQADKVLYALADPVTAKWLKELNATAEALPYNRNNQWRRDTYQEMTERILTDVRKGLNVCAVFYGHPGVFANPAQAAIKQAHREGFTAQMLPGISAEDCLFADLGVDPGRNGCQSFEATDFLIRRRKFDPTSPLILWQIAMVGNLGFYKPEEQFRPLTILTEVLKTHYGGDHEVIVYEAAVYYPVCKPVIQRIPLSKLPQSSVTEVSTLYIPPQ

SEQ ID NO: 198 - protein, 238 aa, Artificial Sequence
  Synthetic
  MISC_FEATURE (1)..(238): Fungal alpha-N-methyltransferase domain shown in FIG. 2A
GSLTVVGTGIQLVGHLTLAAKAWIEQADKVLFAVADPVTAKWLQTLNPTAEALPYNTNNQRRRETYREMVDRILAEVRQGLNVCAVFYGHPGVFAYPTHEAIKQARHEGFRAQMLPGISAEDCLFADLGIDPGRNGCQSFEATDFLIRRRKFDPTSALILWQIATIGNLGFYQEEIQLRGLNVLAEVLETYYGSDHEVIVYEAAVYYPVCEPVIQHIPLSKLAEIRVTLVSTLYVPPQ

SEQ ID NO: 199 - protein, 238 aa, Artificial Sequence
  Synthetic
  MISC_FEATURE (1)..(238): Fungal alpha-N-methyltransferase domain shown in FIG. 2A
GSLTVVGTGIQLVGHLTLAARSWIEGADKVLFAVADPLTAKWLQSLNPAAESLPYDTGNLRRRKTYDEMVGRILVEVRSGLNVCAVFYGHPGVFADPAHEAIRQARREGFRAEMLPGISAEDCLFADLGVDPGRNGCQSFEATDFLIRRRKFDPTSVLILWQIGFTGNLGFFKENAHLRGLKVLAEVLQTDYGPEHEVTVYEAAVYYPVCSPTIERMRLCELPNASVTPVSTLYVPPK

SEQ ID NO: 200 - protein, 238 aa, Artificial Sequence
  Synthetic
  MISC_FEATURE (1)..(238): Fungal alpha-N-methyltransferase domain shown in FIG. 2A
GSLTIVGTGIQLGGHLTLAAQAWIKQADKLLFAVADPVTAKWLQSLNPTAEALPYNTDCDRRRQTYGKMVDRMMEAVRAGGNICAVFYGHPGVFADPAHGAIAQARREGYTALMLPGISAEDCLFADIGIDPGKGGCQSFEATDFLIRRRKFDPNSHLILWQVALIGNLGFYQEGSEQRGLKILAEVLQENYPSEHEVVIYEAAVYYPVCQPVINPIPLDSLPLAKVTTVSTLYVPPL

SEQ ID NO: 201 - protein, 238 aa, Artificial Sequence
  Synthetic
  MISC_FEATURE (1)..(238): Fungal alpha-N-methyltransferase domain shown in FIG. 2A
GCLTIVGTGIQFVGQVTLAAKAWIEQADKVLYAVADPATAEWLRSLNETAESLPYNTNNQWRKQTYGEMVEVILNAVRQGLDVCAVFYGHPGVFANPTREAIRQAREEGFRAQMLPGISASDCLYADVGVDPGQYGCQSFEATDFLLRDRRFDPRSALILWQIGLIGNFSFFDAQGVFPGLKVLTEVLEKHYDRDHEVIVYEAAIYYPVCQPIIEPMPLSKLPEALVSEVSTLYVPPQ

SEQ ID NO: 202 - protein, 240 aa, Artificial Sequence
  Synthetic
  MISC_FEATURE (1)..(240): Fungal alpha-N-methyltransferase domain shown in FIG. 2A
GSLVVVGIGIQWAGQTTLAAQRAIQQADRVLFAVADAWAARWVRSLNPAAESLPYGKGKTPRRSIYAEMVERILAEVRKGLKVCAVFYGHPGVLASPAHAAVRQARDEGFSARMLPGVSSIDCLFADLGVDPGRDGLQIFEATDFLIRGRAVDARTPLVLCQIGGVGCAGVFDPSDAARIRRGLEALGERLRQSYPAAHEVVIYEAAAHPLASPRLDRVALSALSAAPVDDVSTLYVPPA

SEQ ID NO: 203 - protein, 238 aa, Artificial Sequence
  Synthetic
  MISC_FEATURE (1)..(238): Fungal alpha-N-methyltransferase domain shown in FIG. 2A
GSLVVAGSGIKGIAHLTQEAAGWIEQADHVVYCVSDPVTDVWIRKHSKSSDDLYRLYGNDKPRIRTYREMTDAMVEPVRRGMNVCGVFYGHPGVFVHPGHAAIRTVREEGYPAMMLPGVSALDCLFADLGVDPSRPGCQELEATDMLLRDRPLLAESHVVVWQAGCVGDLGFNFAGFTNKHLGVLAEYLDRFYPPDHLITHYEASQYPVCAPHIRTLPLRELRQEPLTGISTLYLPPI

SEQ ID NO: 204 - protein, 220 aa, Artificial Sequence
  Synthetic
  MISC_FEATURE (1)..(220): Fungal alpha-N-methyltransferase domain shown in FIG. 2A
ETKGWIQQSDVVLYCVADPTTEVWIKQNSKFSVDLYQFYGNDRRRLDTYIDMSDEMLKYVRQGKSVCGVFYGHPGVFVYPSHRAVRIARDEGHKAAILPAISALDCLYADCDVDPSSVGSQTVEATDLLLRSRQLLTDEHVVIWQIGCVGDLGFNFSGYDNRHLNILVDYLEKFYDADHEVVHYVGSQYPMCPAIVQRMPLKDLRTASVTGLSTLYIPPQ

SEQ ID NO: 205 - protein, 238 aa, Artificial Sequence
  Synthetic
  MISC_FEATURE (1)..(238): Fungal alpha-N-methyltransferase domain shown in FIG. 2A
AELIVVGTGYRAVGDLTLEAKACLEQADTVLCLVGDPMVIGYLERLNPSVRTLAGFYAKGKHRSESYEEMVQEILSEVRRERLVCCAFYGHPGVFAYPGHEAVRRARLEGIPARMLAGSSAEDWLFADLGLDPGTCGCQSFEASDFLLRRRVFDATSLLVLWQVGVIGMTDRDPDFDARVGAALLAERLAGAYGVDHEVVVYEASPYPVTSPRISPVPLAKLADTQLTNKSTLVVPPL

SEQ ID NO: 206 - protein, 238 aa, Artificial Sequence
  Synthetic
  MISC_FEATURE (1)..(238): Fungal alpha-N-methyltransferase domain shown in FIG. 2A
AQLVVVGTGYRAIGDLTVEARACIEQADKVLCLIGDPLVTHHIGQLNPSVETLDGHYAAGKPRYASYEEMVEHILSEVRRDQFVCVALYGHPGVFAYAGHEAIRRARLEGIAARMVPACSAEDWLFADLGLDPGQQGCQSFEATDFLTRHRRFDPTSLMILWQIGVIGMPDAHRQFDPAPGASCLTEVLVPHYGPDHPVTVYEASPYVAAEPRATTVPLAKLPDTSLSAASTLVVPPL

SEQ ID NO: 207 - protein, 238 aa, Artificial Sequence
  Synthetic
  MISC_FEATURE (1)..(238): Fungal alpha-N-methyltransferase domain shown in FIG. 2A
AQLVVVGTGFRAIGDLTVEARACLEQADKVLCLIGDPLVTRHIEKLNASVETLDVHYAVGKPRSASYEDMVEHIMSELHRDQFVCVALYGHPGVFAYTGHEAIRRAREEGIAARMLPACSAEDWLFADLGLDPGERGCQSFEATDFLIRHRVFDPTGLLILWQVGVIGMIDRDPGYDARPGVTTLTDALVASYGSGHPVTVYEASPYVTAEPRTTTVPLAELPDTPLSAASTLVVPPL

SEQ ID NO: 208 - protein, 242 aa, Artificial Sequence
  Synthetic
  MISC_FEATURE (1)..(242): Fungal alpha-N-methyltransferase domain shown in FIG. 2A
GSLIVVGTGIRAGHLSQEAISAVRTADAVAYCIAEPLTRLLVDELRKEAAKPVAEDLHRFYADGKPRTQTYHQMVARILELVRLDLQVAAVFYGHPGVFAYPSHESIRQARAEGYPAEMLPGISAVDCLFADLGVDPATAGCQMLEATDFLLRRRHLDTACGVVLWQIGCVGHGDYQGSGYDLRYIPMLVEALLAFYPPEHEVVVYQAAHFAMCDPIVECVAIAEMLKASITAVSTLYIPPL

SEQ ID NO: 209 - protein, 238 aa, Artificial Sequence
  Synthetic
  MISC_FEATURE (1)..(238): Fungal alpha-N-methyltransferase domain shown in FIG. 2A
GSLVVVGTGLHVGQLTLESRANIEVADHVVYLVTDSVTEEFILSLNSNAESLRQYYADDKNRLISYEEMIAHITNLVHNNLEVCAVFYGHPGIFVYPSHESIQRLRQEGYKARMLPGISAEDCLYADLGIDPGRQGSQSFEATDFILRDRIFDPHSYLILWQIGSLGSYTFSSTGIYDRRGVDILLNKLLSNYPSNHEVILYKASVIPLCSHEVQRFALCDLVTAKTNGSMTLIIPPC

SEQ ID NO: 210 - protein, 242 aa, Artificial Sequence
  Synthetic
  MISC_FEATURE (1)..(242): Fungal alpha-N-methyltransferase domain shown in FIG. 2A
GTLVLMGSGIRAIGQFTLESQEYLRWADRVFYCVSDPTTERWILDQQPDAVDLYTLYDNDKPRRITYVQMAESMLRPLREGLNVVGVFYGHPGVFVNPSHRAIAIARQEGHAAFMLPAVSALDCLFSDLGIDPSRPGCQIIEATDLLLRKRQLLTDSHVVLLQVGSVGDTGFRFAGFPNTHLGALVNYLQEAYGPDHEIVNYIAAQYAIADPVVQRVKLSELLEPENSRRVTGISTFYVPPV

SEQ ID NO: 211 - protein, 241 aa, Artificial Sequence
  Synthetic
  MISC_FEATURE (1)..(241): Fungal alpha-N-methyltransferase domain shown in FIG. 2A
GRLVVIGSGIKAVSHFTLEAQQHIRNADIVLYAAADPVTDIWIQKQNKNSFDLYQYYADDKSRIITYSQMIERTMQEVRAGKYVCALFYGHPGVFVTPSHNAIAIARSEGYHAVMLPAISAEDCLYADLGVDPSVPGLQIYEATDFLLRRRHIDTSANFVLWQVGCIGDLGFKFGGYQNDKLDVLLDYLEEIYGPDHIAINYVANMFSGEPTIDRHRIADYRDPEVKRTVSGISTFFLPAK

SEQ ID NO: 212 - protein, 242 aa, Artificial Sequence
  Synthetic
  MISC_FEATURE (1)..(242): Fungal alpha-N-methyltransferase domain shown in FIG. 2A
GKLIVIGSGIKSIAHFTLESQAHLQQADIVLYAASDPVTDMWIQKQNPNSFDLYQYYGNTKNRIITYTQMIERVMMELRSGKYVCALFYGHPGVFVTPSHNAIELARREGYEAEMLPGISAEDCLFADLGVDPSIPGLQTYEATDLLLRQRSINTEINAVIWQVGCVGDVGFKFHGYDNEKLNILLDYLDKFYPPDQIVYNYVASMFSMAKPKKDKFKLSDFRDPSIAKEVTGISTFFIPAV

SEQ ID NO: 213 - protein, 242 aa, Artificial Sequence
  Synthetic
  MISC_FEATURE (1)..(242): Fungal alpha-N-methyltransferase domain shown in FIG. 2A
GHLVIVGSGIKSLAQLTMEAVGHIKAADEVFYAVADPLTEAFIEKNAKKAYDMYILYDDGKPRLQTYVQMAEVMLRALRQGKYVVGVFYGHPGVFVNPSHRAIAIAKEEGFRAEMLAGISAEDNLFADIGIDPSRHGCQTLEATDLLLRKRILLTDSHVVILQVGAVGDLGFNFSGFKNQHFQVLIDRLIKEYGPQHDVYLYAAPSIAIANPLVEKYKIADFRKPEVVKRVTGISTFYLPPK

SEQ ID NO: 214 - protein, 241 aa, Artificial Sequence
  Synthetic
  MISC_FEATURE (1)..(241): Fungal alpha-N-methyltransferase domain shown in FIG. 2A
GELTIIGSGIETMGFTIGDEELIRGADAVFFCVADPATVVWLKSIRPDAYDLYVLYDDTKVRYTTYMQMSEAMLHHVRQGKKVVAVYYGHPGIFVLSTHRCIMIARREGHKAVMRPGVCALDCLCADLGVDPCHPGMQTHEATDMLIRGRIPDTSLHVVLWQVGLIGEMGFRRRGYINNNFSVFVEYLQKYYGDDYPVTHYIASRYPTIPPTIEVYPLSALHDPQIQTRVTGVSTFYVPPK

SEQ ID NO: 215 - protein, 241 aa, Artificial Sequence
  Synthetic
  MISC_FEATURE (1)..(241): Fungal alpha-N-methyltransferase domain shown in FIG. 2A
GELTIIGSGIETVGFSLGDQELIRSADHVFYCVADPATVVWIRDLRPDAHDLYVLYDDNKVRYVTYMQMAEAMLHHVRQAKKVVAIYYGHPGVFVLSTHRAILIARREGHRATMRAGVSALDCLCADLGVDPSHPGLQTHEASDMLIRLRKPDTSLHVVLWQVGLIGEMGFRRKGYVNSNFSILIDYLQGVYGKDYPVTHYVASRYPTIPPLIETYPLSRLHEPDIQLRVTGISTFYLAPR

SEQ ID NO: 216 - protein, 241 aa, Artificial Sequence
  Synthetic
  MISC_FEATURE (1)..(241): Fungal alpha-N-methyltransferase domain shown in FIG. 2A
GELIIIGSGIETIGISLGDQKLIEAADKVLFCVADPATIVWLKRLRPDALDLYVLYGENKVRYTTYMQMTEAQLYWVRQGLKVVVVFYGHPGIFVLSTHRAIKIARREGYKAVMKAGVCALDTLCADIGIDPCHPGLQTHEATDALVRQRNLDTSLHVVLWQVGLIGELGFRRQGYLNNDFSYFISWLQNIYGEEYKVTHYIGSRYPTIPPLIEIYHLNEMHDPETQLKITGLSTFYIPPR

SEQ ID NO: 217 - protein, 241 aa, Artificial Sequence
  Synthetic
  MISC_FEATURE (1)..(241): Fungal alpha-N-methyltransferase domain shown in FIG. 2A
GSLEILGSGIEASGFTRADEARIRAADHVFFCVADPATKVWLLRERPDAYDLYTLYDDSKRRYVTYMQMAEAILHPVRQGQRVVAIFYGHPGVFVLATHRAVRIARREGHRAEMRAAVSALDTLCADLGVDPSQPGMQMYEATDMLIRSRRPDTGLHLVLWQVGLIGELGYRRQGYLNSNFSVLLDYLEALYGADQPVVNYVGSRYPGVDPLIDRQTIASLRDPSVQNWVTGISTFYLPPK

SEQ ID NO: 218 - protein, 241 aa, Artificial Sequence
  Synthetic
  MISC_FEATURE (1)..(241): Fungal alpha-N-methyltransferase domain shown in FIG. 2A
GTLEILGSGIEASDFSRSDEARILVADHVFYCVADPATKIWILSQRPDAYDLYVLYDDSKPRYLTYMQMTEAMLHHVRNGEHVVAIFYGHPGVFVLSTHRAVTIARREGHHASMRAAVSALDTLCADLGVDPSQPGMQMYEATDMLIRRRQPDPGLHLVLWQVGLIGELGYRRQGYLNSNFAVLLDYLEDLYGPEHPVINYVGSRYPGIDPLIDRQTLASLRDPLAQSWVTGISTFYLPPR

SEQ ID NO: 219 - protein, 241 aa, Artificial Sequence
  Synthetic
  MISC_FEATURE (1)..(241): Fungal alpha-N-methyltransferase domain shown in FIG. 2A
GELTILGSGIETVGFTSADEIRIREADKVFYCVADPATVVWLKRLRPDAYDLYVLYDDTKIRYTTYMQMTEAMLHFVREGQNVVAIFYGHPGVFVLSTHRAVQIGQREGHKVTMRAGISALDTLCADLGIDPSQPGMQTFEATDTLIRKRHLDPELHLILWQVGLVGDLGYRREGSLNSGFSVLLDYLEETYGPDHEVVNYIGSRYPGADPVRDRHTISSLRNPAVQSTITGISTFYIPPA

SEQ ID NO: 220 - protein, 241 aa, Artificial Sequence
  Synthetic
  MISC_FEATURE (1)..(241): Fungal alpha-N-methyltransferase domain shown in FIG. 2A
GDLTIIGSGIEAVGFTSVDESLIKRADYVFFCVADPATSVWIKSIRPDAYDLYVLYDDSKLRYLTYIQMSEAILHYVRRGAKVVAIYYGHPGVFVFSTHRAVQIARREGHKAVMRANVSALDTLCADLGVDPSQPGMQTFEATDMLIRQRQIDTGLHLILWQVGLIGELGYRRQGYLNNSFSVLLDYIEEAYGSDYPIVNYIGSRYPGIDPTKDQHTVSSLRVPEAQSTVTGISTFYIPPK

SEQ ID NO: 221 - protein, 241 aa, Artificial Sequence
  Synthetic
  MISC_FEATURE (1)..(241): Fungal alpha-N-methyltransferase domain shown in FIG. 2A
GELTVLGSGIEAAGFTRADERLLREADHVFHCVADPVTVVRIKAWRPDSYDLGVLYDDAKDRYLTYVQMSEAILHFVRAGRRVAAVFYGHPGVFVLSSHRAVRIARREGHAAVMRPGISALDVLCADLGVDPSSPGMQTFEASDLLIRGRRIDPGLHLVLWQVGLVGELGYQRTGFFNHRLPVLVDRLEEVYGPDHPVVNYIGARYRGTAPVTDRHTVAGLRDRAVQRTVTSASTWYLPPA

SEQ ID NO: 222 - protein, 241 aa, Artificial Sequence
  Synthetic
  MISC_FEATURE (1)..(241): Fungal alpha-N-methyltransferase domain shown in FIG. 2A
GELTIIGSGIGVMGFTRDAEQYIDDADHVVFCVADPATQVWLRGRRPDAIDLFALYDDRKPRYHTYMQMTEAMLYHVRRGKRVAAVFYGHPGIFVLSTHRAVAIARREGHRAQLRPGVSALDWLCADLGIDPAYPGMQTFEATDMLLRRRAIDPGSHVVLWQVGLIAEMGFRRRGFHNRRFDYLVEYLRGYYKPDHKIVHYIASRYPTLPPVIERYTLDELQNPEVHALITGISTFYVPPA

SEQ ID NO: 223 - protein, 243 aa, Artificial Sequence
  Synthetic
  MISC_FEATURE (1)..(243): Fungal alpha-N-methyltransferase domain shown in FIG. 2A
GSLTVVGTGLRALSHMTLEAISHIRDADRVFFSVPDGVTARQIRDINPEAVDLTQYYGEDKRRKQTYVQMSEVILREVRAGSAVTAVFYGHPGFFVFPARRILSIARKEGYRAVMLPGISSLDCLMADLRVDPSVNGCQILEATDLLLRNRPIITSGHVIILQVGSVGDSAFSFTAGFRHAKRAVLFERLIEAYGEEHRSVLYLAATYPGLDGQAVVRPLGAYRDPKVLASVPPAGTLYIPAK

SEQ ID NO: 224 - protein, 242 aa, Artificial Sequence
  Synthetic
  MISC_FEATURE (1)..(242): Fungal alpha-N-methyltransferase domain shown in FIG. 2A
GSLTVVGTGIRAVSHLTQEAIGHIRDADAVFYSVPDGITAAYIRDLNPNATNLSVLYGEDKRRSVTYVQMAEVILRAVRQGKTVVAVFYGHPGYFVNPARRALTIARKEGHATAMLPGISSTDCLFADLRIDPSFQGCQILEATDYLLRDRPLVTSGHVVLLQVGSVGDAYFSYGGFKNSKRAVLFERLIETYGPDHPCVHYVGATFPGTDPQVTRRPLGAYRDPKVLAGIHYACSLYIPPK

SEQ ID NO: 225 - protein, 241 aa, Artificial Sequence
  Synthetic
  MISC_FEATURE (1)..(241): Fungal alpha-N-methyltransferase domain shown in FIG. 2A
GDLIILGAGIASVGFTMDAEMYIRRADSVFYVVTDPVTEVWINKLRPDAYDLSTLYNDNKPRYNTYMQMTEAMLYDVRQGRTVVGIFYGHPGIFALSTHRAIMIARREGHRAVMKPGISALDCLCADLGVDPSYPGMQTMEATDMLLRGKKPDTTQHVILWQVGLIGIYDFRRRGFINKNFNIFIRFLQNVYGEDYSITHYVAASFPTLAPLIQNFTLRELLYPDIQAKVQSMSTFYLAPK

SEQ ID NO: 226 - protein, 243 aa, Artificial Sequence
  Synthetic
  MISC_FEATURE (1)..(243): Fungal alpha-N-methyltransferase domain shown in FIG. 2A
KKLIIAGTGIKFLSHLTIEVKSAIETSCCVVFLLNEPAMKNWVVKNAKKYVSLDDIYFSSEQRSESYDKIANELLSTIQKNDDVCFLIYGHPTFFSSVVEKIKKIISSEDIIIQIMPGISAMDCLFADLKIDPGSMGLQSYDATEFLLYDENFSTTSHLVLWQIAIIGEIGVVNNNEINLDRQKKAITILIKKLLIHYPADHIVYLYVASQYPSIEFELVSIDLQKLTEINISRLATLYIPPV

SEQ ID NO: 227 - protein, 207 aa, Artificial Sequence
  Synthetic
  MISC_FEATURE (1)..(207): Fungal alpha-N-methyltransferase domain shown in FIG. 2A
YLVNEPITKQWIERYSKLSESLDPIYFAENDRQISYDKIRDKILTELETHNFITVVLYGHPTVFADPGLQSIIIAQKKSIETIVLPGISVENCLYADLKIDPGQFGCFHVEATELLLYDKIIDPTVHLCIWQPGMIGNRSVPKPNQKSNHLELLRIKLKKYYPDDHISILYEASMYPGVEPIIHKFPLYDIEDQNIGTLSTLYIPPL

SEQ ID NO: 228 - protein, 241 aa, Artificial Sequence
  Synthetic
  MISC_FEATURE (1)..(241): Fungal alpha-N-methyltransferase domain shown in FIG. 2A
GSLAIVGTGIQALGHLTREAEGHIRHADVVFHCIRDPLMLADLHALNPNVIDLTTLYGADKRRSLTYVQMAEVMVAEVRSGRRVTGVFYGHPGFYVAPARLALTTAKAEGYDTCLLPGVSSLDYLFADLRVDPSQSGYQAMEATDFLLRNRPLVTSGHVVLMQVGPVGDLFRPVTDNLERRAILFERLIEIYGENHPCVDYVGATRPGQKPVLRRLKLGAYRDPANLKRLSKASTLYIPPK

SEQ ID NO: 229 - protein, 241 aa, Artificial Sequence
  Synthetic
  MISC_FEATURE (1)..(241): Fungal alpha-N-methyltransferase domain shown in FIG. 2A
GRLVVIGSGIKAVSHFTLEAQAHIQQADIVLYAAADPVTDMWIESQNPNAFDLYQYYADDKARLITYVQMIERIMAEVRAGKYVCALFYGHPGVFVTPSHNAIAIARQEGFDAVMLPAVSAEDCLYADLGVDPSVPGMQIYEATDFLLRRRKVDTTANFVLWQVGCIGDLGFKFGGYKNDKFDVLLDYLEEIYGADHPAINYVANMFSGPPQIDRHVIGDYRDPEVKAKVSGISTFFIPAK